(19) United States
(12) Patent Application Publication
(10) Pub. No.: US 2015/0011893 A1
(43) Pub. Date: Jan. 8, 2015

(54) EVALUATION OF SKIN LESIONS BY RAMAN SPECTROSCOPY

(71) Applicant: THE UNIVERSITY OF BRITISH COLUMBIA, Vancouver (CA)

(72) Inventors: Harvey Lui, Vancouver (CA); Haishan Zeng, Vancouver (CA); David I. McLean, Vancouver (CA); Jianhua Zhao, Burnaby (CA); Michael Schulzer, Vancouver (CA)

(73) Assignee: The University of British Columbia, Vancouver (CA)

(21) Appl. No.: 14/357,288

(22) PCT Filed: Nov. 7, 2012
(86) PCT No.: PCT/CA2012/050790

§ 371 Date: May 9, 2014

Related U.S. Application Data
(60) Provisional application No. 61/557,853, filed on Nov. 9, 2011.

(51) Int. Cl. A61B 5/00

(52) U.S. Cl. CPC A61B 5/444; A61B 5/0075
USPC 600/476

(57) ABSTRACT

A Raman spectrometer system provides a tool for discriminating between different tissue pathologies. The tool may provide discrimination indicators for a plurality of different pairs of tissue pathologies. Improved sensitivity and specificity are achieved by basing discriminations on appropriate ranges within a Raman spectrum.

### CROSS REFERENCE TO RELATED APPLICATIONS

This application claims priority from U.S. Application No. 61/557,853 filed 9 Nov. 2011 and entitled EVALUATION OF SKIN LESIONS BY RAMAN SPECTROSCOPY. For purposes of the United States, this application claims the benefit under 35 U.S.C. §119 of U.S. Application No. 61/557,853 filed 9 Nov. 2011 and entitled EVALUATION OF SKIN LESIONS BY RAMAN SPECTROSCOPY, which is hereby incorporated herein by reference for all purposes.

### TECHNICAL FIELD

The invention relates to the evaluation of skin lesions. The invention may be applied, for example, to provide methods and apparatus for assessing skin lesions. An example embodiment provides an apparatus which may be used by a physician or other medical professional to evaluate the likelihood that skin lesions are cancerous or pre-cancerous and/or to classify skin lesions (for example to distinguish malignant melanoma from seborrheic keratosis).

### BACKGROUND

Skin cancer is common. On average, about one in every five North Americans will eventually develop malignant skin tumors. When a suspicious lesion is detected by a physician, biopsy followed by histopathologic examination is the most accurate way to confirm a diagnosis. This process is invasive, time consuming and can be associated with some morbidity. The importance of achieving high diagnostic sensitivity necessitates a low threshold for biopsy, which in turn incurs higher costs for the health care system. Furthermore, a biopsy alters the site under study and can leave permanent scars. In some cases the most appropriate site to biopsy can be difficult to ascertain.

A difficulty in evaluating lesions is that there are a variety of benign lesions that can visually mimic skin cancer. These include pathologies such as: seborrheic keratosis (SK), atypical nevi (AN), melanocytic nevi (which include the varieties junctional (JN), compound (CN), and intradermal (IN)), and blue nevi (BN).

A sensitive, specific, non-invasive tool for characterizing suspicious lesions and other tissues would provide a valuable alternative to the use of biopsies and histopathologic examination of tissues extracted by biopsy to evaluate skin lesions.

Raman spectroscopy involves directing light at a specimen which inelastically scatters some of the incident light. The inelastic interactions between the light and the specimen can cause the scattered light to have wavelengths that are shifted relative to the wavelength of the incident light (Raman shift). The wavelength spectrum of the scattered light (the Raman spectrum) contains information about the nature of the specimen. The Raman spectrum is typically specified in terms of the amount of Raman shift measured in cm⁻¹.

Raman spectroscopy is sensitive to molecular vibrations and provides “fingerprint” signatures for various biomolecules in tissue such as proteins, lipids and nucleic acids. Raman spectroscopy is capable of detecting subtle molecular or biochemical changes associated with tissue pathology. However, the probability that any given incident photon will undergo Raman scattering is exceedingly low, making it particularly challenging to measure. Until recently Raman spectroscopy systems suitable for use on human subjects were very slow. Such systems could take many minutes to obtain a Raman spectrum for a single location. This slowness was one factor that limited the application of Raman spectroscopy in clinical settings. Multi-channel charge-coupled device (CCD) based dispersive Raman systems can simultaneously detect Raman signals of different wavelengths. This substantially reduces integration times required to obtain Raman spectra.

The use of Raman spectroscopy in the study of tissues is described, inter alia, in the following references:
A. Caspers P J, et al. Raman spectroscopy in biophysics and medical physics. Biophys J 2003; 85:572-580;
B. Huang Z, et al. Rapid near-infrared Raman spectroscopy system for real-time in vivo skin measurements. Opt Lett 2001; 26:1782-1784;
C. Short M A, et al. Development and preliminary results of an endoscopic Raman probe for potential in vivo diagnosis of lung cancers. Opt Lett 2008; 33(7):711-713;
D. Huang Z, et al. Raman spectroscopy of in vivo cutaneous melanin. J of Biomed Opt 2004; 9:1198-1205;
E. Huang Z, et al. Raman Spectroscopy in Combination with Background Near-infrared Autofluorescence Enhances the In Vivo Assessment of Malignant Tissues. Photochem Photobiol 2005; 81:1219-1226;
F. Molckovsky A, et al. Diagnostic potential of near-infrared Raman spectroscopy in the colon: differentiating adenomatous from hyperplastic polyps. Gastrointest Endosc 2003; 57:396-402;
G. Abigail S H, et al. In vivo Margin Assessment during Partial Mastectomy Breast Surgery Using Raman Spectroscopy. Cancer Res 2006; 66:3317-3322;
H. Rajadhyaksha M, et al. In Vivo Confocal Scanning Laser Microscopy of Human Skin II: Advances in Instrumentation and Comparison With Histology. J Invest Dermatol 1999; 113:293-303;
Lieber C A, et al. In vivo nonmelanoma skin cancer diagnosis using Raman micro spectroscopy. Laser Surg Med 2008; 40(7):461-467;
J. Caspers P J, et al. Automated depth-scanning confocal Raman micro spectrometer for rapid in vivo determination of water concentration profiles in human skin. J Raman Spectrosc 2000; 31:813-818;
K. Caspers P J, et al. In vivo confocal Raman microspectroscopy of the skin: noninvasive determination of molecular concentration profiles. J Invest Dermatol 2001; 116:434-442;
L. Caspers P J, et al. Monitoring the penetration enhancer dimethyl sulfoxide in human stratum corneum in vivo by confocal Raman spectroscopy. Pharm Res 2002; 19:1577-1580;
M. Lieber, C. A. et al., Raman microspectroscopy for skin cancer detection in vitro, J. Biomed. Opt. 13, 024013 (2008);
N. A. Nijssen et al., Discriminating basal cell carcinoma from its surrounding tissue by Raman spectroscopy, J. Invest. Dermatol. 119, 64-69 (2002);
O. A. Nijssen et al., Discriminating basal cell carcinoma from perilesional skin using high wave-number Raman spectroscopy, J. Biomed. Opt. 12, 034004 (2007);
P. M. Gniadecka, et al., Melanoma diagnosis by Raman spectroscopy and neural networks: structure alterations in proteins and lipids in intact cancer tissue, J. Invest. Dermatol. 122, 443-449 (2004);
Q. M. Gniadecka et al., Diagnosis of basal cell carcinoma by Raman spectroscopy, J. Raman Spectrosc. 28, 125-129 (1997);
R. P. J. Caspers, et al., In vivo confocal Raman Microspectroscopy of the skin: noninvaisve determination of molecular concentration profiles, J. Invest. Dermatol. 116, 434-442 (2001);
S. A. C. Williams, et al., Fourier transform Raman spectroscopy: a novel application for examining human stratum corneum, Int. J. Pharm. 81, R11-R14 (1992)
T M. Mogensen et al., Diagnosis of nonmelanoma skin cancer/keratinocyte carcinoma: a review of diagnostic accuracy of nonmelanoma skin cancer diagnostic tests and technologies, Dermatol. Surg. 33, 1158-1174 (2007);
U. A. A. Marghoob, et al., Instruments and new technologies for in vivo diagnosis of melanoma, J. Am. Acad. Dermatol. 49, 777-797 (2003);
V. J. Zhao, et al., Quantitative analysis of skin chemicals using rapid near-infrared Raman spectroscopy, Proc. SPIE 6842, 684209 (2008);
W J. Zhao, et al., Integrated real-time Raman system for clinical in vivo skin analysis, Skin Res. and Tech. 14, 484-492 (2008);
X. H. Zeng, et al. Skin cancer detection using in vivo Raman spectroscopy in SPIE Newsroom (2011), DOI: 10.1117/2.1201104.003705;
Y. J. Zhao, et al., Real-time Raman spectroscopy for non-invasive skin cancer detection—preliminary results, EMBS 2008, 3107-3109 (2008);
Z. H. Zeng, et al., Raman spectroscopy for in vivo tissue analysis and diagnosis, from instrument development to clinical applications, Journal of Innovation in Optical Health Sciences 1, 95-106 (2008).
   All of these references are hereby incorporated herein by reference.

There remains is a need for practical sensitive and specific methods for screening for skin cancers such as melanomas and for classifying abnormal tissues. There is a specific need for methods and apparatus useful for 1) discriminating skin cancers and precancers from benign skin lesions; 2) discriminating malignant melanoma from other non-melanoma pigmented lesions; and, 3) discriminating malignant melanoma from seborrheic keratosis.

### SUMMARY OF THE INVENTION

This invention has a number of aspects. These aspects include: apparatus useful for assessing the pathology of tissue (e.g. skin) in vivo; methods useful for assessing the pathology of tissue (e.g. skin) in vivo; apparatus for processing tissue Raman spectroscopy data and generating a measure of the likelihood that the spectra correspond to cancerous or pre-cancerous tissues; methods for processing tissue Raman spectroscopy data and generating a measure of the likelihood that the spectra correspond to cancerous or pre-cancerous tissues; non-transitory media containing computer-readable instructions that, when executed by a data processor cause the data processor to execute a method for processing tissue Raman spectroscopy data and generating a measure of the likelihood that the spectra correspond to cancerous or pre-cancerous tissues. Methods and apparatus that are operable to distinguish cancerous or pre-cancerous skin lesions from benign lesions and/or seborrheic keratosis.

One aspect of the invention provides an apparatus for tissue characterization comprising a Raman spectrometer configured to generate a Raman spectrum and a Raman spectrum analysis unit configured to measure at least one characteristic of the Raman spectrum and to perform classification of tissue based on the at least one characteristic of the Raman Spectrum.

In some embodiments the apparatus is configured to discriminate malignant melanoma from seborrheic keratosis or other pigmented lesions. In some embodiments the Raman spectrometer is configured to obtain a complete or partial Raman spectrum in the wavenumber range of 1055-1800(cm⁻¹) and the Raman spectrum analysis unit is configured to measure the at least one characteristic of the Raman spectrum from the complete or partial Raman spectrum in the wavenumber range of 1055-1800(cm⁻¹). In some embodiments the apparatus is configured to perform a principal component analysis (PCA) linear discriminant analysis (LDA) or a PLS analysis on the acquired Raman spectrum.

Another aspect of the invention provides a method for discriminating malignant melanoma from seborrheic keratosis or other pigmented lesions. In some embodiments the method comprises obtaining a complete or partial Raman spectrum in the wavenumber range of 1055-1800(cm⁻¹) and acquiring at least one characteristic of the acquired Raman spectrum. In some embodiments the method comprises performing a PCA-LDA or PCA-GDA analysis on the acquired Raman spectrum.

Another aspect of the invention provides a method for evaluating pathology of a living tissue. The method comprises obtaining a Raman spectrum for the tissue; deriving a first indicator indicating which of a first pair of pathologies the tissue is most likely to be affected by based on a first range of the Raman spectrum; and, deriving a second indicator indicating which of a second pair of pathologies the tissue is most likely to be affected by based on a second range of the Raman spectrum different from the first range.

Another aspect of the invention provides a method for generating an indicator indicating whether a tissue is more likely affected by cancer or actinic kereosis, on the one hand, and benign lesions, on the other hand. The method comprises obtaining a Raman spectrum for at least a majority of the following 11 sub-ranges: 500-513, 546-586, 611-675, 721-736, 760-830, 870-900, 947-1320, 1345-1420, 1434-1457, 1478-1520, and 1540-1790 cm⁻¹; and, generating the indicator based upon the values of the Raman spectrum in the sub-ranges while excluding values of the Raman spectrum outside of the sub-ranges.

Another aspect of the invention provides a method for generating an indicator indicating whether a tissue is more likely affected by cancer, on one hand, and benign lesions, on the other hand. The method comprises obtaining a Raman spectrum for at least a majority of the following sub-ranges 500-511, 546-584, 618-675, 721-1210, 1232-1288, 1351-1422, 1468-1500, 1533-1681, and 1693-1800 cm⁻¹; and, generating the indicator based upon the values of the Raman spectrum in the sub-ranges while excluding values of the Raman spectrum outside of the sub-ranges.

Another aspect of the invention provides a method for generating an indicator indicating whether a tissue is more likely affected by malignant melanoma, on one hand, and other pigmented lesions, on the other hand. The method comprises obtaining a Raman spectrum for at least a majority of the following sub-ranges 1055-1100, 1292-1322, 1357-1414, 1426-1480, 1617-1644, 1672-1721, and 1769-1787 cm⁻¹; and, generating the indicator based upon the values of the Raman spectrum in the sub-ranges while excluding values of the Raman spectrum outside of the sub-ranges.

Another aspect of the invention provides a method for generating an indicator indicating whether a tissue is more likely affected by malignant melanoma, on one hand, and seborreic keratosis, on the other hand. The method comprises obtaining a Raman spectrum for at least a majority of the following sub-ranges: 1055-1106, 1143-1147, 1255-1263, 1288-1322, 1343-1416, 1428-1497, 1591-1649, 1665-1736, and 1760-1791 cm⁻¹; and generating the indicator based upon the values of the Raman spectrum in the sub-ranges while excluding values of the Raman spectrum outside of the sub-ranges.

Another aspect of the invention provides a method for tissue evaluation. The method comprises obtaining a Raman spectrum for the tissue; and processing the Raman spectrum by a computer to generate an indicator indicating whether the tissue is more likely affected by malignant melanoma, on one hand, or seborreic keratosis, on the other hand.

Another aspect of the invention provides a method for characterising a tissue. The method comprises obtaining a Raman spectrum of the tissue in a spectral range including at least 1055 cm⁻¹ to 1800 cm⁻¹; processing by a data processor that portion of the Raman spectrum lying between 1055 cm⁻¹ and 1800 cm⁻¹ to yield an indicator indicative of a pathology of the tissue, the indicator not based on any portion of the Raman spectrum outside of the range of about 1000 cm⁻¹ to 1900 cm⁻¹.

Another aspect of the invention provides a method for characterising a tissue. The method comprises processing by a computer a Raman spectrum of the tissue. The processing comprises determining weights for a plurality of components of the Raman spectrum and applying a discrimination function to the weights wherein the components are components that have been determined from reference Raman spectra for lesions having a plurality of known tissue pathologies without reference to Raman spectra for normal skin.

Another aspect of the invention provides a method for differentiating pigmented skin lesions from non-pigmented skin lesions. The method comprises measuring the Raman spectrum of the tissue at 1745, 1655, 1620, and 1370 cm⁻¹; and deriving an indicator indicating whether a skin lesion is more likely to be a pigmented skin lesion or a non-pigmented skin lesion based on the Raman spectrum.

Another aspect of the invention provides a method for evaluating pathology of a living tissue. The method comprises obtaining a Raman spectrum for the tissue; decomposing the Raman spectra of the tissue into components corresponding to the Raman spectra of specific molecules and/or other moieties found in tissues; and applying a discrimination function to the weights of these components.

Another aspect of the invention provides apparatus for evaluating pathology of a living tissue. The apparatus comprises a processor configured to process a Raman spectrum for the tissue. The processor is operative to derive a first indicator indicating which of a first pair of pathologies the tissue is most likely to be affected by based on a first range of the Raman spectrum and to derive a second indicator indicating which of a second pair of pathologies the tissue is most likely to be affected by based on a second range of the Raman spectrum different from the first range. In some embodiments the first range includes Raman shifts between about 500 and 1800 cm⁻¹.

Another aspect of the invention provides apparatus for generating an indicator indicating whether a tissue is more likely affected by cancer or actinic kereosis, on the one hand, and benign lesions, on the other hand. The apparatus comprises a processor configured to process a Raman spectrum for at least a majority of the following 11 sub-ranges: 500-513, 546-586, 611-675, 721-736, 760-830, 870-900, 947-1320, 1345-1420, 1434-1457, 1478-1520, and 1540-1790 cm⁻¹. The processor is configured to generate the indicator based upon the values of the Raman spectrum in the sub-ranges while excluding values of the Raman spectrum outside of the sub-ranges.

Another aspect of the invention provides apparatus for generating an indicator indicating whether a tissue is more likely affected by cancer, on one hand, and benign lesions, on the other hand. The apparatus comprises a processor configured to process a Raman spectrum for at least a majority of the following sub-ranges 500-511, 546-584, 618-675, 721-1210, 1232-1288, 1351-1422, 1468-1500, 1533-1681, and 1693-1800 cm⁻¹. The processor is configured to generate the indicator based upon the values of the Raman spectrum in the sub-ranges while excluding values of the Raman spectrum outside of the sub-ranges.

Another aspect of the invention provides apparatus for generating an indicator indicating whether a tissue is more likely affected by malignant melanoma, on one hand, and other pigmented lesions, on the other hand. The apparatus comprises a processor configured to process a Raman spectrum for at least a majority of the following sub-ranges 1055-1100, 1292-1322, 1357-1414, 1426-1480, 1617-1644, 1672-1721, and 1769-1787 cm⁻¹. The processor is configured to generate the indicator based upon the values of the Raman spectrum in the sub-ranges while excluding values of the Raman spectrum outside of the sub-ranges.

Another aspect of the invention provides apparatus for generating an indicator indicating whether a tissue is more likely affected by malignant melanoma, on one hand, and seborreic keratosis, on the other hand. The apparatus comprises a processor configured to process a Raman spectrum for at least a majority of the following sub-ranges: 1055-1106, 1143-1147, 1255-1263, 1288-1322, 1343-1416, 1428-1497, 1591-1649, 1665-1736, and 1760-1791 cm⁻¹. The processor is configured to generate the indicator based upon the values of the Raman spectrum in the sub-ranges while excluding values of the Raman spectrum outside of the sub-ranges.

Another aspect of the invention provides apparatus for tissue evaluation. The apparatus comprises a processor configured to process a Raman spectrum for the tissue to generate an indicator indicating whether the tissue is more likely affected by malignant melanoma, on one hand, or seborreic keratosis, on the other hand.

In some embodiments processing the Raman spectrum comprises determining principal component (PC) scores for a plurality of predetermined PCs and applying a general determinant analysis to the PC scores. In some embodiments the Raman spectrum covers the range of 1055 cm⁻¹ to 1800 cm⁻¹. In some embodiments the principal components include four or more, or two or more, principal components as shown in FIGS. 7A-7E. In some embodiments, the four or more, or the two or more, PCs include the first four, or first two, PCs as shown in FIG. 7A.

In some embodiments processing the Raman spectrum comprises determining partial least squares (PLS) factor scores for a plurality of predetermined PLS factors. In some embodiments the Raman spectrum covers the range of 500 cm⁻¹ to 1800 cm⁻¹. In some embodiments the PLS factors include four or more, or two or more, PLS factors as shown in FIGS. 16A-16E. In some embodiments, the four or more, or the two or more, PLS factors include the first four, or first two, PLS factors as shown in FIG. 16A.

Another aspect of the invention provides apparatus for tissue evaluation. The apparatus comprises a processor configured to process a Raman spectrum for the tissue to generate an indicator indicating whether the tissue is more likely affected by one pathology, on the one hand, or another pathology, on the other hand.

In some embodiments processing the Raman spectrum comprises determining principal component (PC) scores for a plurality of predetermined principal components and applying a general determinant analysis to the PC scores. In some embodiments the Raman spectrum covers the range of 500 cm⁻¹ to 1800 cm⁻¹. In some embodiments the Raman spectrum covers the range of 1055 cm⁻¹ to 1800 cm⁻¹. In some embodiments the principal components include two or more (or four or more in some cases) principal components as shown in one or more of FIG. 7A-7E, or 8A-8E, or 9A-9E, or 10A-10E, or 11A-11E, or 12A-12E. In some embodiments, the four or more, or the two or more, PCs include the first four, or first two, PCs as shown in FIG. 7A, or 8A, or 9A, or 10A, or 11A, or 12A. The set of PCs used is selected to correspond with the discrimination between pathologies to be performed.

In some embodiments processing the Raman spectrum comprises determining partial least squares (PLS) factor scores for a plurality of predetermined PLS factors. In some embodiments the Raman spectrum covers the range of 500 cm⁻¹ to 1800 cm⁻¹. In some embodiments the Raman spectrum covers the range of 1055 cm⁻¹ to 1800 cm⁻¹. In some embodiments the PLS factors include four or more, or two or more, PLS factors as shown in FIG. 13A-13E, or 14A-14E, or 15A-15E, or 16A-16E, or 17A-17E, or 18A-18E. In some embodiments, the four or more, or the two or more, PLS factors include the first four, or first two, PLS factors as shown in FIG. 13A-13E, or 14A-14E, or 15A-15E, or 16A-16E, or 17A-17E, or 18A-18E. The set of PLS factors used is selected to correspond with the discrimination between pathologies to be performed.

Another aspect of the invention provides apparatus for characterising a tissue. The apparatus comprises a processor configured to process a Raman spectrum for the tissue to generate indicators for one or more discriminations. The indicators indicate whether the tissue is more likely affected by one pathology, on the one hand, or another pathology, on the other hand. In some embodiments, the apparatus operated by determining PLS factor scores for a plurality of predetermined PLS factors, and/or determining PC scores for a plurality of predetermined PCs and applying a general determinant analysis to the PC scores (or PLS factor scores). In some embodiments, the apparatus can be set by a user to distinguish between a particular pair of pathologies. In some embodiments, when set to distinguish between pathology A and pathology B, the device uses predetermined PLS factors and/or the predetermined PCs associated with that pair of pathologies. In some embodiments, the predetermined PLS factors include PLS factors as shown in one or more of FIGS. 13A-13E, 14A-14E, 15A-15E, 16A-16E, 17A-17E, and 18A-18E. In some embodiments, the predetermined PCs include PCs as shown in one or more of FIGS. 7A-7E, 8A-8E, 9A-9E, 10A-10E, 11A-11E, and 12A-12E.

Another aspect of the invention provides apparatus for characterising a tissue. The apparatus comprises a processor configured to process that portion of a Raman spectrum of the tissue in a spectral range lying between 1055 cm⁻¹ to 1800 cm⁻¹ to yield an indicator indicative of a pathology of the tissue. The apparatus is configured such that the indicator is not based on any portion of the Raman spectrum outside of the range of about 1000 cm⁻¹ to 1900 cm⁻¹.

Another aspect of the invention provides apparatus for characterising a tissue, the apparatus comprising a processor configured to process a Raman spectrum of the tissue. The processing comprises determining weights for a plurality of components of the Raman spectrum and applying a discrimination function to the weights wherein the components are components that have been determined from reference Raman spectra for lesions having a plurality of known tissue pathologies without reference to Raman spectra for normal skin. In some embodiments the components comprise principal components. In some embodiments the components comprise partial least squares factors.

Another aspect of the invention provides a system for evaluating tissue pathology. The system comprises a processor configured to access a Raman spectrum of a tissue to be studied. The processor is configured (for example by way of software instructions) to determine from the Raman spectrum a first indicator for differentiating one or more of cancer and actinic kereosis from benign lesions using a first range within the Raman spectrum and to determine a second indicator for differentiating melanoma from non-melanoma pigmented lesions and/or melanoma from sebhorreic keratosis using a second range within the Raman spectrum that is smaller than the first range. In one example embodiment the first range is about 500 cm⁻¹ to 1800 cm⁻¹ and the second range is about 1055 cm⁻¹ to 1800 cm⁻¹.

Another aspect of the invention provides apparatus and methods as described herein. As will be apparent to those of skill in the art, features of different illustrative embodiments described herein may be combined to yield further example embodiments. Further, non-essential features of the illustrative embodiments described herein may be omitted to provide other example embodiments. Thus the invention encompasses apparatus comprising any new and useful feature, combination of features or sub-combination of features present in any embodiment disclosed herein or any combination of embodiments disclosed herein. The invention also encompasses methods that include any new and useful act, step, combination of acts and/or step or sub-combination of acts present in any embodiment disclosed herein or any combination of embodiments disclosed herein.

Additional aspects of the invention and features of example embodiments of the invention are described in the following description and/or illustrated in the accompanying drawings.

### BRIEF DESCRIPTION OF THE DRAWINGS

The accompanying drawings illustrate non-limiting embodiments of the invention.

FIG. 1 is a block diagram of a diagnostic apparatus according to an example embodiment of the invention.

FIG. 1A is a schematic view of one example of a user interface arranged to communicate indicators to a user.

FIG. 2 is a block diagram of an apparatus according to another example embodiment of the invention.

FIGS. 3A, 3B and 3C respectively show a raw Raman spectrum, the Raman spectrum of FIG. 3A with a polynomial curve fit to the fluorescence background and the Raman spectrum of FIG. 3A with the fluorescence background as modelled by the polynomial curve subtracted.

FIG. 4 is a flow chart illustrating a method according to an example embodiment of the invention.

FIGS. 4A through 4D show example Raman spectra for various moieties found in tissues that may be applied as reference molecules.

FIG. 5 shows mean Raman spectra for different skin pathologies.

FIG. 6 is a flow chart of an example spectral analysis method configured to obtain indicators for two different discriminations based on a Raman spectrum.

FIGS. 7A through 7E show principal components generated for discriminating malignant melanoma (MM) from seborrheic keratosis (SK) using Raman spectra in the range of 1055 cm⁻¹ to 1800 cm⁻¹. FIG. 7F illustrates how the explained variances increases with the number of PC factors used.

FIGS. 8A through 8E show principal components generated for discriminating malignant melanoma (MM) from seborrheic keratosis (SK) using Raman spectra in the range of 500 cm⁻¹ to 1800 cm⁻¹. FIG. 8F illustrates how the explained variances increases with the number of PC factors used.

FIGS. 9A through 9E show principal components generated for discriminating cancer and pre-cancer (e.g. actinic keratosis—AK) from non-cancer using Raman spectra in the range of 500 cm⁻¹ to 1800 cm⁻¹. FIG. 9F illustrates how the explained variances increases with the number of PC factors used.

FIGS. 10A through 10E show principal components generated for discriminating cancer and pre-cancer (AK) from non-cancer using Raman spectra in the range of 1055 cm⁻¹ to 1800 cm⁻¹. FIG. 10F illustrates how the explained variances increases with the number of PC factors used.

FIGS. 11A to 11E show principal components generated for discriminating MM from non-melanoma pigmented lesions using Raman spectra in the range of 1055 cm⁻¹ to 1800 cm⁻¹. FIG. 11F illustrates how the explained variance increases with the number of principal components used.

FIGS. 12A to 12E show principal components generated for discriminating MM from non-melanoma pigmented lesions using Raman spectra in the range of 500 cm⁻¹ to 1800 cm⁻¹. FIG. 12F illustrates how the explained variance increases with the number of principal components used.

FIGS. 13A to 13E show PLS factors generated for discriminating MM from non-melanoma pigmented lesions using Raman spectra in the range of 1055 cm⁻¹ to 1800 cm⁻¹. FIG. 13F illustrates how the explained variance increases with the number of PLS regression components used.

FIGS. 14A to 14E show PLS factors generated for discriminating MM from non-melanoma pigmented lesions using Raman spectra in the range of 500 cm⁻¹ to 1800 cm⁻¹. FIG. 14F illustrates how the explained variance increases with the number of PLS regression components used.

FIGS. 15A to 15E show PLS factors generated for discriminating MM from SK using Raman spectra in the range of 1055 cm⁻¹ to 1800 cm⁻¹. FIG. 15F illustrates how the explained variance increases with the number of PLS regression components used.

FIGS. 16A to 16E show PLS factors generated for discriminating MM from SK using Raman spectra in the range of 500 cm⁻¹ to 1800 cm⁻¹. FIG. 16F illustrates how the explained variance increases with the number of PLS regression components used.

FIGS. 17A to 17E show PLS factors generated for discriminating cancer and pre-cancer (AK) from non-cancer using Raman spectra in the range of 500 cm⁻¹ to 1800 cm⁻¹. FIG. 17F illustrates how the explained variance increases with the number of PLS regression components used.

FIGS. 18A to 18E show PLS factors generated for discriminating cancer and pre-cancer (AK) from non-cancer using Raman spectra in the range of 1055 cm⁻¹ to 1800 cm⁻¹. FIG. 18F illustrates how the explained variance increases with the number of PLS regression components used.

FIG. 19 is a receiver operating characteristic (ROC) plot for the results of an analysis to discriminate between cancer and pre-cancer, on one hand, and benign lesions, on the other hand using PCA-GDA.

FIG. 20 shows the posterior probability for each measured lesion to be classified as a skin cancer or precancer.

FIG. 21 is a ROC plot for discrimination between MM and pigmented benign lesions with 95% CI. At a sensitivity of 90%, the overall specificity is over 64%, with a positive predictive value (PPV) of 67% and a negative predictive value (NPV) of 89%. The estimated biopsy ratio is 0.5:1.

FIG. 22 shows the posterior probability for each measured lesion to be classified as malignant melanoma.

FIG. 23 is a ROC plot for a discrimination between malignant melanoma (MM) and non-melanoma pigmented lesions (AN, BN, CN, IN, JN, SK) based on Raman spectra for lesions located on the head only.

FIG. 24 is a ROC curve for a discrimination between malignant melanoma (MM) and non-melanoma pigmented lesions (AN, BN, CN, IN, JN, SK) using differences between the Raman spectra for the lesions and adjacent normal skin.

FIG. 25 is a ROC curve for discrimination between biopsied malignant melanoma (MM) from biopsied non-melanoma pigmented lesions (AN, BN, CN, IN, JN, SK).

FIG. 26 is a ROC curve for a discrimination between malignant melanoma (MM) and non-melanoma pigmented lesions (AN, BN, CN, IN, JN, SK) based on Raman spectra derived from biopsied lesions only.

### DESCRIPTION

Throughout the following description, specific details are set forth in order to provide a more thorough understanding of the invention. However, the invention may be practiced without these particulars. In other instances, well known elements have not been shown or described in detail to avoid unnecessarily obscuring the invention.

Accordingly, the specification and drawings are to be regarded in an illustrative, rather than a restrictive, sense.

### Example System Architecture

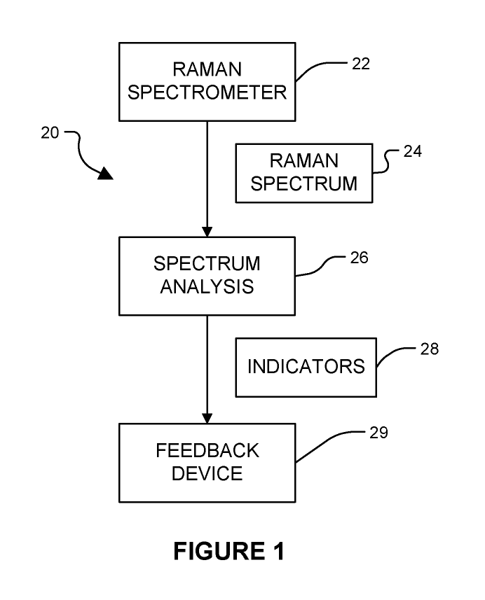

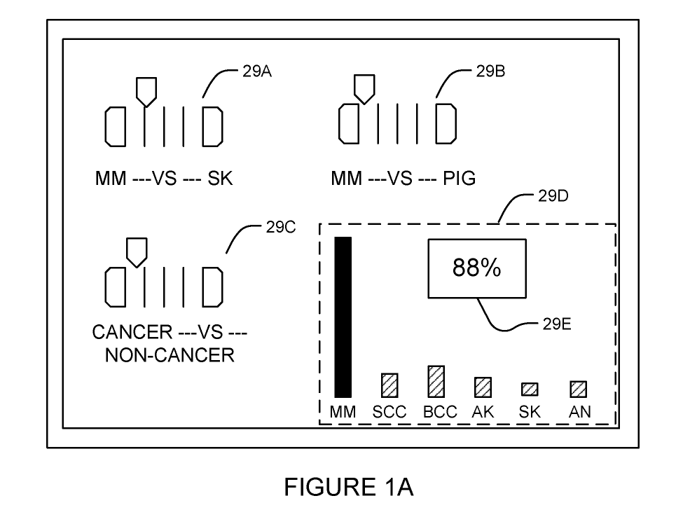

FIG. 1 is a block diagram of apparatus 20 according to an example embodiment of the invention. Apparatus 20 comprises a Raman spectrometer 22 which is configured to determine a Raman spectrum 24 for a small volume of a tissue. The tissue may be skin, for example. Raman spectrometer 22 is preferably configured to obtain Raman spectra over at least the range 500 cm⁻¹ to 1800 cm⁻¹.

A spectrum analysis component 26 receives Raman spectrum 24 and processes the Raman spectrum to obtain one or more indicators 28. Indicator(s) 28 are indicative of the pathology of the tissue for which Raman spectrum 24 was obtained. Indicators 28 are displayed, stored, transmitted and/or applied to control a human-perceptible signal by a feedback device 29.

Indicators 28 may, for example, indicate a discrimination between different tissue types such as: cancer/pre-cancerous lesions vs benign lesions; malignant melanoma vs. non-melanoma pigmented lesions; and/or malignant melanoma vs. seborrheic keratosis. Such indicators may indicate a confidence level that the tissue is of one of the types being discriminated (e.g. a value on a scale in which one end of the scale represents a high likelihood that the tissue is of one type and the other end of the scale represents a high likelihood that the tissue is of the second type). Intermediate values of such an indicator may indicate varying degrees of certainty that the tissue is of one or the other of the types being discriminated. Indicators 28 may also, or in the alternative, indicate classifications of lesion tissues into different pathologies such as normal, malignant melanoma, basal cell carcinoma, squamous cell carcinoma, pre-cancerous lesions such as actinic keratosis, and benign skin lesions such as sebhorreic keratosis, atypical nevi, etc. Indicators 28 may also, or in the alternative, indicate whether or not intervention is suggested to the physician, e.g. biopsy or treatment of the lesion.

FIG. 1A is a schematic view of one example of a user interface 29 arranged to communicate indicators 28 to a user. Display features 29A through 29C indicate discrimination between different pairs of tissue types. Display feature 29D indicates a classification. Display feature 29E indicates a degree of confidence in the classification.

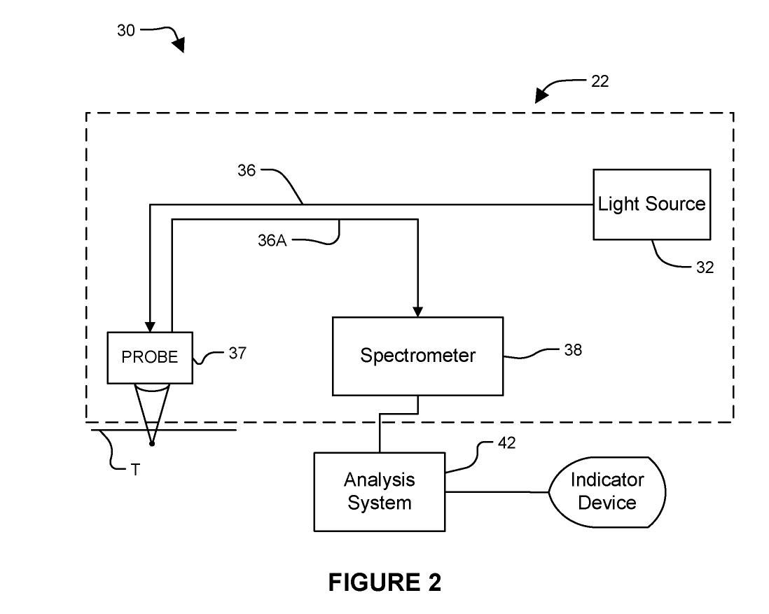

FIG. 2 is a block diagram of apparatus 30 according to another example embodiment of the invention. In FIG. 2, Raman spectrometer 22 is shown to comprise a light source 32. Light source 32 is a monochromatic light source and may, for example, comprise a laser. Light source 32 may, for example, comprise a wavelength stabilized diode laser operating at a suitable wavelength such as 785 nm.

Apparatus 30 comprises a fiber/fiber bundle delivery system 36, a Raman probe 37, a spectrometer 38, and a spectrum analysis system 42 interfaced to receive data from spectrometer 38. In an example embodiment, light from light source 32 is delivered to Raman probe 37 through a 200-μm core-diameter single fiber. At Raman probe 37 the light is collimated, filtered by a 785 nm band pass filter, and delivered to illuminate an area of skin tissue T with a diameter of 3.5 mm.

It is desirable to avoid exposing tissues to excessive amounts of radiation. This may be achieved by appropriate selection of light source, control of the light source, and/or providing attenuation downstream from the light source. The intensity of light issuing from Raman probe 37 may be controlled so that the irradiance on skin does not exceed the American National Standards Institute (ANSI) maximum permissible exposure (MPE) level (e.g. 1.63 W/cm²).

The raw signal, which includes a Raman scattering signal and a tissue autofluorescence background is collected by Raman probe 37 and transmitted to spectrometer 38 through a fiber bundle 36A. In an example embodiment, fiber bundle 36A comprises fifty-eight 100 μm core-diameter low-OH fibers. Such fibers advantageously provide high transmission of near infrared NW light. A distal end of fiber bundle 36A that connects to Raman probe 37 is packed into a 1.3-mm diameter circular area. A proximal end of fiber bundle 36A that is connected to deliver light to spectrometer 38 is designed such that the fiber tips are aligned along a parabolic line that is in an inverse orientation to the image aberration of the transmissive spectrograph.

Spectrometer 38 measures a spectrum of the light. In an example embodiment, spectrometer 38 is equipped with an NIR-optimized back-illumination deep-depletion CCD array (LN/CCD-1024EHRB, Princeton Instruments, Trenton, N.J.) and a transmissive imaging spectrograph (HoloSpec-f/2.2-NIR, Kaiser Optical Systems Inc., Ann Arbor, Mich.) with a holographic grating (HSG-785-LF, Kaiser Optical Systems Inc., Ann Arbor, Mich.). In an example system the CCD array has a 16 bit dynamic range and is liquid nitrogen cooled to −120° C.

In some embodiments, one or more fibers, for example a center fiber, may be used for calibration. The fibers may be arranged so that their image is symmetrical along a centerline of the CCD detectors. With this fiber arrangement, image aberration can be fully corrected. This facilitates full-chip vertical hardware binning. The spectral resolution of an example system used to acquire Raman spectra for the trials reported below is 8 cm⁻¹.

A spectral analysis system 42 analyzes spectra from spectrometer 38. Spectral analysis system 42 is configured to identify specific spectral characteristics of Raman spectra received from spectrometer 38. Apparatus comprising a stand-alone spectral analysis system 42 provides another example application of the invention.

Spectral analysis system 42 may comprise a programmed data processor such as a personal computer, an embedded computer, a microprocessor, a graphics processor, a digital signal processor or the like executing software and/or firmware instructions that cause the processor to extract the specific spectral characteristics from the Raman spectra. In alternative embodiments spectrum analysis system 42 comprises electronic circuits, logic pipelines or other hardware that is configured to extract the specific spectral characteristics or a programmed data processor in combination with hardware that performs one or more steps in the extraction of the specific spectral characteristics.

In a prototype embodiment, spectral analysis system 42 comprises a personal computer, embedded computer or workstation executing software that provides calibration procedures and real-time data processing, including intensity calibration and fluorescence background removal.

It is convenient but not mandatory for spectral analysis system 42 to operate in real time or near real time such that analysis of a Raman spectrum is completed at essentially the same time or at least within a few seconds of the Raman spectrum being acquired by scanning a subject. A stand-alone spectral analysis system 42 may acquire Raman spectra data from scans done in the past and/or be connected to a Raman spectrometer to process a Raman spectrum in real time as the Raman spectrum is obtained by scanning a subject.

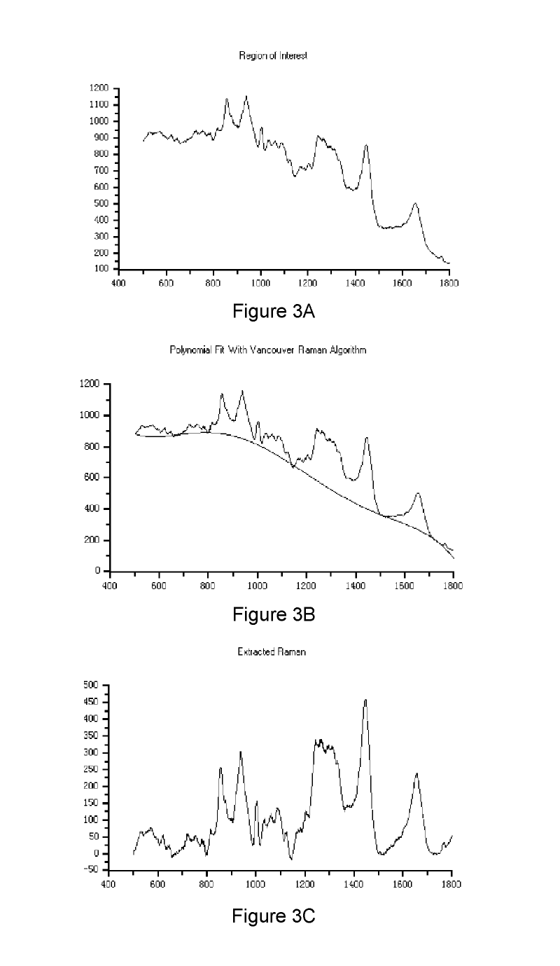

Measured Raman spectra are typically superimposed on a fluorescence background, which varies with each measurement. It is convenient for spectral analysis system 42 to process received spectra to remove the fluorescence background and also to normalize the spectra. Removal of fluorescence background may be achieved, for example using the Vancouver Raman Algorithm as described in Zhao J, et al. Automated Autofluorescence Background Subtraction Algorithm for Biomedical Raman Spectroscopy. Appl. Spectrosc. 2007; 61:1225-1232, which is hereby incorporated herein by reference. The Vancouver Raman Algorithm is an iterative modified polynomial curve fitting fluorescence removal method that takes noise into account. FIGS. 3A, 3B and 3C respectively show a raw Raman spectrum, the Raman spectrum of FIG. 3A with a polynomial curve fit to the fluorescence background and the Raman spectrum of FIG. 3A with the fluorescence background as modelled by the polynomial curve subtracted.

Spectral analysis system 42 may normalize Raman spectra. For example, the area under curve (AUC) of each spectrum may be set to a standard value. For example, spectral analysis system 42 may multiply each spectrum by a value selected to make the AUC equal to the standard value. For convenience in displaying the spectra, the normalized intensities may optionally be divided by the number of data points in each spectrum.

As described below, through analysis of the Raman spectra it is possible to discriminate cancer/pre-cancerous lesions from benign lesions. It is also possible to discriminate malignant melanoma from non-melanoma pigmented lesions. It is also possible to discriminate malignant melanoma from seborrheic keratosis. Methods and apparatus as described herein may also be used to classify lesion tissue into a wide range of pathologies such as normal, malignant melanoma, basal cell carcinoma, squamous cell carcinoma, pre-cancerous lesions such as actinic keratosis, and benign skin lesions such as sebhorreic keratosis, atypical nevi, etc.

### Raman Spectrum Analysis

It is a challenge to extract from Raman spectra information that is useful for tissue classification and/or tissue differentiation. It is a particular challenge to obtain such information that can provide reliable indicators of tissue classification and/or tissue differentiation. Two approaches to analyzing Raman spectral data are to identify and directly measure specific features in a Raman spectrum and to apply multivariate data analysis. These approaches can provide equivalent results.

Examples of multivariate data analysis are principal components analysis (PCA) followed by general discriminant analysis (GDA) which may in some cases be linear discriminant analysis (LDA) and least squares analysis for example partial least squares (PLS). An example of PLS is the nonlinear iterative partial least squares (NIPALS) algorithm. For example, a particular spectrum may be analyzed by performing PCA+GDA and/or a least squares analysis. Any of these techniques may be performed on part or all of the range of the acquired Raman spectra.

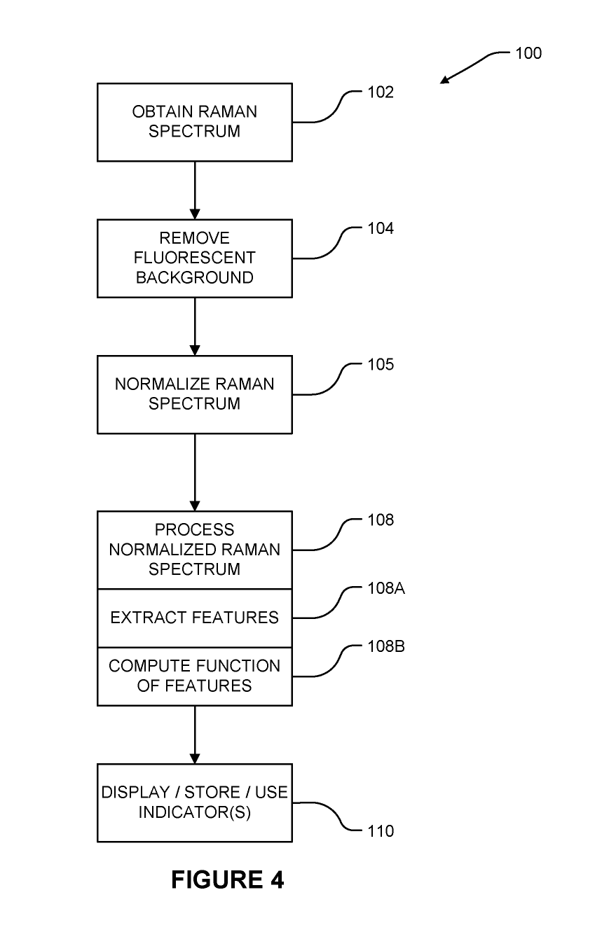

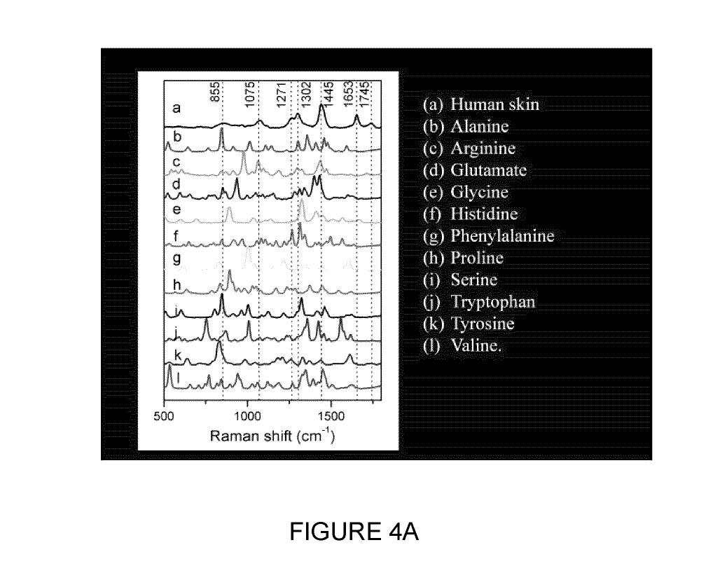

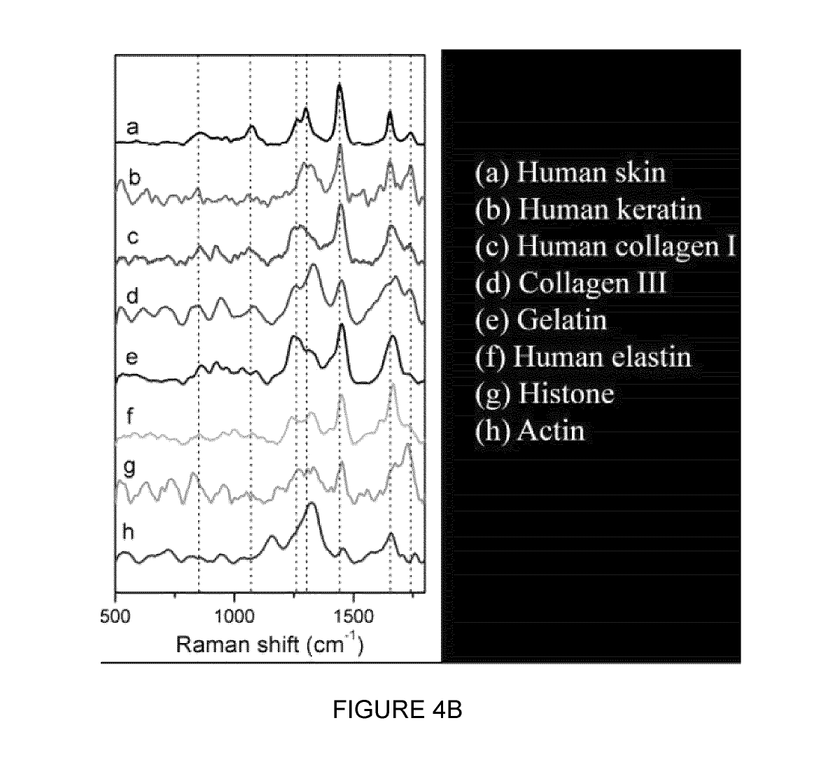

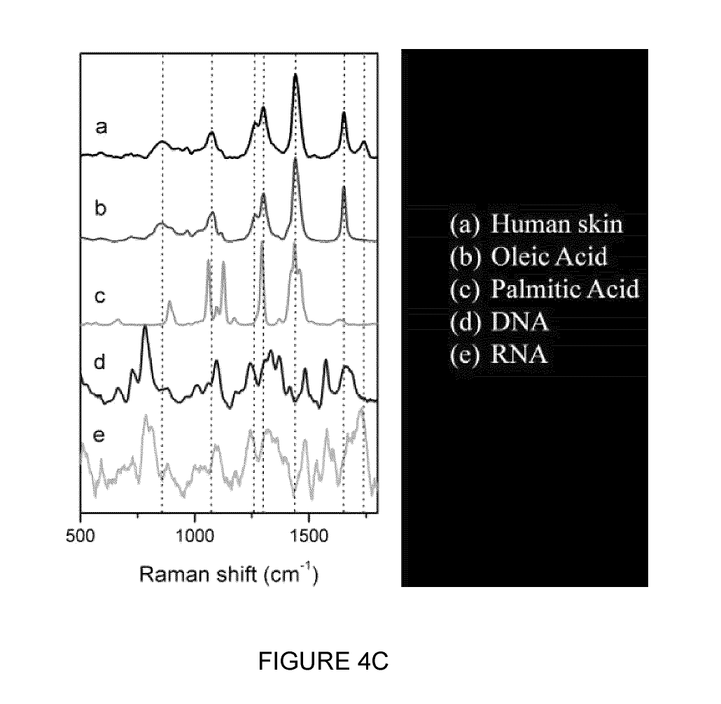

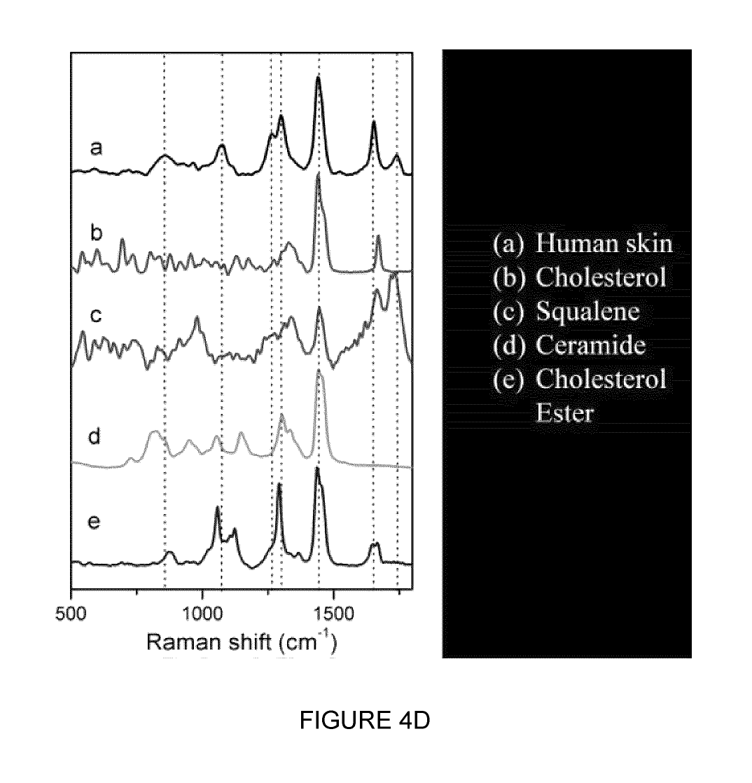

Another embodiment decomposes Raman spectra into components corresponding to the Raman spectra of specific molecules and other moieties found in tissues and applies a discrimination function to the weights of these components. Such embodiments may be called “reference-molecule-based”. For example, PLS may be applied to the component weights. FIGS. 4A through 4D show example Raman spectra for various moieties found in tissues including: alanine, argenine, glutamate; glycine, histidine, phenylalanine, proline, serine, tryptophan, tyrosine, valine, human leratin, collagen I, collagen III, gelatin, human elastone, histone, actin, oleic acid, palmitic acid, DNA, RNA, cholesterol, squalene, ceramide, and cholesterol ester. In some embodiments, discrimination is based in whole or part on weights corresponding to some or all of these moieties in a Raman spectrum.

PCA involves generating a set of principal components which represent a given proportion of the variance in a set of training spectra. For example, each Raman spectrum may be represented as a linear combination of a set of a few (e.g. 3 to 20) PCA variables. The PCA variables may be selected such that they represent at least a threshold amount (e.g. 70%) of the total variance of the set of training spectra.

Principal components (PCs) may be derived by performing PCA on the standardized spectral data matrix to generate PCs. The PCs generally provide a reduced number of orthogonal variables that account for most of the total variance in original spectra.

PCs may be used to assess a new Raman spectrum by computing a variable called the PC score for each PC. The PC score represents the weight of that particular component (PC factor) in the Raman spectrum being analyzed.

General discriminant analysis (of which linear discriminant analysis is an example) can then be used to derive a function of the PC scores which can be used to classify or discriminate tissue types. The discriminate function surface or curve may subsequently be applied to categorize an unknown tissue pathology based on where a point corresponding to the PC scores for a Raman spectrum of the unknown tissue is relative to the discriminate function surface or curve.

A PLS algorithm determines values for the matrices B and E such that the matrix equation:

Y=XB+E (1)

holds where Y is a matrix of responses (here the responses are indicators of one or more discriminations, X is a matrix containing predictors (here Raman spectra of lesions or values derived from Raman spectra of lesions). B and E can be obtained by applying a PLS algorithm to the known values for the predictors and responses from a data set such as that described in Table I. Once B and E have been determined, a response (e.g. a discrimination indicator) can be predicted for a new set of predictors (e.g. a Raman spectrum for a lesion being investigated).

In some embodiments the predictors comprise integrated values of the Raman spectrum in a plurality of sub-ranges of the Raman spectrum. In some embodiments the predictors comprise measured values of the Raman spectrum. In some embodiments the predictors comprise the weights in the Raman spectrum for a plurality of reference moieties.

Leave-one-out cross validation may be used to verify that a discrimination technology such as a set of principal components and the associated discriminate function or a set of PLS factors provides a suitably sensitive and specific test for tissue pathology.

The receiver operating characteristic (ROC) curve is one way to illustrate the balance of sensitivity versus specificity. A ROC curve may be calculated from the posterior probabilities obtained in leave-one-out cross validation. The determination of ROC curves is described, for example, in the following references which are hereby incorporated herein by reference: J. A. Hanley and B. J. McNeil, The meaning and use of the area under a receiver operating characteristic (ROC) curve, Radiology 143, 29-36 (1982); J. A. Hanley and B. J. McNeil, A method of comparing the areas under receiver operating characteristic curves derived from the same cases, Radiology 148, 839-843 (1983); C. E. Metz, et al. Maximum likelihood estimation of receiver operating characteristic (ROC) curves from continuously-distributed data., Statistics in Medicine 17, 1033-1053 (1998); K. O. Hajian-Tilaki, et al. A comparison of parametric and nonparametric approaches to ROC analysis of quantitative diagnostic tests, Medical Decision Making 17, 94-102 (1997); D. Bamber, The area above the ordinal dominance graph and the area below the receiver operating graph., Journal of Mathematical Psychology 12, 387-415 (1975).

With good discrimination between two groups of tissue pathology the ROC curve moves toward the left and top boundaries of the graph, whereas poor discrimination yields a curve that approaches a diagonal line. The AUC of an ROC curve ranges from 0.5 in a case where discrimination performance is the same as random chance to 1.0 representing perfect discrimination.

FIG. 4 illustrates a method 100 according to an example embodiment of the invention. Method 100 operates a Raman spectrometer to obtain a Raman spectrum of a subject's tissue in block 102. Block 102 may be performed with a probe that is held against the skin of a living subject.

In block 104 the fluorescent background is removed from the Raman spectrum. In block 105 the Raman spectrum is normalized.

In block 108 the normalized Raman spectrum is processed to evaluate one or more indicators. The indicators are displayed, stored and/or otherwise communicated at block 110.

Processing the Raman spectrum may comprise extracting features of the Raman spectrum as indicated at block 108A and calculating a function of the extracted features as indicated at block 108B. The function calculated in block 108B may be determined so as to achieve the desired discrimination and/or classification on a set of training data. The training data may comprise Raman spectra from tissues affected by a wide range of known pathologies. In one example embodiment, block 108A comprises determining PC scores for a set of principal components and block 108B comprises computing a function of the PC scores.

### Reference Data

Data used to develop and calibrate prototype methods and apparatus described herein were obtained in a study of patients presenting with skin lesions. Patients over 18 years of age having lesions of potential concern for skin cancer or having incidental skin lesions of clinical interest were considered for the study.

Raman spectra for each patient were recorded and stored in a database for analysis. Raman spectra were obtained for both lesional skin and adjacent normal-appearing skin. The “normal” skin measurement site was usually within 5 cm of the visible border of the target skin lesion. To take a Raman measurement the handheld spectrometer probe was placed to contact the target skin site gently without compressing the skin. An integration time of one second was used for acquiring the Raman spectra. The patients underwent spectral measurements of up to 10 separate skin lesions. Each lesion was separately diagnosed. Lesions were not considered for analysis if they were less than 1 mm in lateral dimension, located at a body site that was inaccessible to the spectrometer probe, were infected, or had previously been biopsied, excised, or traumatized. The final diagnosis for each measured lesion was established through (1) clinical evaluation by one of two experienced dermatologists, and/or (2) histopathologic analysis if a skin biopsy of the lesion was taken subsequent to the optical measurements.

The database includes Raman spectra of lesions from over 1000 patients. The lesions included both cancerous and benign skin lesions. In the database are Raman spectra from lesions confirmed to be malignant melanoma (MM) 44 cases, basal cell carcinoma (BCC) 109 cases, squamous cell carcinoma (SCC) 47 cases, atypical nevi (AN) 57 cases, blue nevi (BN) 13 cases, compound nevi (CN) 30 cases, intradermal nevi (IN) 38 cases, junctional nevi (JN) 34 cases, seborrheic keratosis (SK) 114 cases and actinic keratosis (AK) 32 cases.

All of the cancerous lesions (MM, BCC, SCC, 100%) were biopsied. Only some of the precancerous lesions (AK, 31%) and benign lesions (AN, BN, CN, IN, JN, SK, 28%) were biopsied. Details of the cohort of the patients, including lesion locations, patient gender and age information are listed in Table I.

TABLE I
Reference Data

| | | # | | # | | Fe- | |
|---|---|---|---|---|---|---|---|
| Pathology | | cases | location | biopsied | Male | male | Age |
| MM | LM | 20 | head 19 | 20 (100%) | 12 | 8 | 69 |
| | | | trunk 1 | | | | (51 to |
| | | | upper limb 0 | | | | 88) |
| | | | lower limb 0 | | | | |
| | LMM | 8 | head 8 | 8 (100%) | 7 | 1 | 67 |
| | | | trunk 0 | | | | (42 to |
| | | | upper limb 0 | | | | 85) |
| | | | lower limb 0 | | | | |
| | SS | 14 | head 3 | 14 (100%) | 6 | 8 | 60 |
| | | | trunk 3 | | | | (22 to |
| | | | upper limb 7 | | | | 77) |
| | | | lower limb 1 | | | | |
| | other | 2 | head 1 | 2 (100%) | 2 | 0 | 61 |
| | | | trunk 1 | | | | (60 to |
| | | | upper limb 0 | | | | 62) |
| | | | lower limb 0 | | | | |
| BCC | super- | 28 | head 10 | 29 (100%) | 14 | 15 | 62 |
| | ficial | | trunk 9 | | | | (34 to |
| | | | upper limb 5 | | | | 86) |
| | | | lower limb 4 | | | | |
| | nodular | 73 | head 52 | 73 (100%) | 41 | 32 | 66 |
| | | | trunk 10 | | | | |
| | | | upper limb 9 | | | | |
| | | | lower limb 2 | | | | |
| | pig- | 6 | head 2 | 6 (100%) | 2 | 4 | 67 |
| | mented | | trunk 4 | | | | |
| | | | upper limb 0 | | | | |
| | | | lower limb 0 | | | | |
| | other | 2 | head 1 | 2 (100%) | 1 | 1 | 68 |
| | | | trunk 1 | | | | |
| | | | upper limb 0 | | | | |
| | | | lower limb 0 | | | | |
| SCC | in situ | 18 | head 7 | 18 (100%) | 13 | 5 | 70 |
| | | | trunk 4 | | | | |
| | | | upper limb 5 | | | | |
| | | | lower limb 2 | | | | |
| | invasive | 28 | head 16 | 28 (100%) | 17 | 11 | 66 |
| | | | trunk 1 | | | | |
| | | | upper limb 5 | | | | |
| | | | lower limb 6 | | | | |
| | other | 1 | head 1 | 1 (100%) | 0 | 1 | 78 |
| | | | trunk 0 | | | | |
| | | | upper limb 0 | | | | |
| | | | lower limb 0 | | | | |

| | | | | | | |
|---|---|---|---|---|---|---|
| SK | 114 | head 47 | 31 (27%) | 65 | 49 | 65 |
| | | trunk 47 | | | | |
| | | upper limb 14 | | | | |
| | | lower limb 6 | | | | |
| AN | 57 | head 3 | 24 (42%) | 29 | 28 | 48 |
| | | trunk 39 | | | | |
| | | upper limb 8 | | | | |
| | | lower limb 7 | | | | |
| BN | 13 | head 4 | 4 (31%) | 4 | 9 | 37 |
| | | trunk 1 | | | | |
| | | upper limb 6 | | | | |
| | | lower limb 2 | | | | |
| CN | 30 | head 9 | 6 (20%) | 14 | 16 | 35 |
| | | trunk 8 | | | | |
| | | upper limb 9 | | | | |
| | | lower limb 4 | | | | |
| IN | 38 | head 21 | 12 (32%) | 9 | 29 | 50 |
| | | trunk 8 | | | | |
| | | upper limb 7 | | | | |
| | | lower limb 2 | | | | |
| JN | 34 | head 5 | 4 (12%) | 12 | 22 | 40 |
| | | trunk 11 | | | | |
| | | upper limb 15 | | | | |
| | | lower limb 3 | | | | |
| AK | 32 | head 28 | 10 (31%) | 16 | 16 | 66 |
| | | trunk 0 | | | | |
| | | upper limb 3 | | | | |
| | | lower limb 1 | | | | |

The Raman spectra were normalized to their respective area-under-the-curve (AUC) before analysis. For most of the lesions only a single Raman spectrum was acquired. For some large and inhomogeneous lesions, particularly for MM (34%) and SCC (17%), up to three Raman spectra were obtained at different locations within the lesion. For lesions with multiple spectra, the average of the normalized spectra was used.

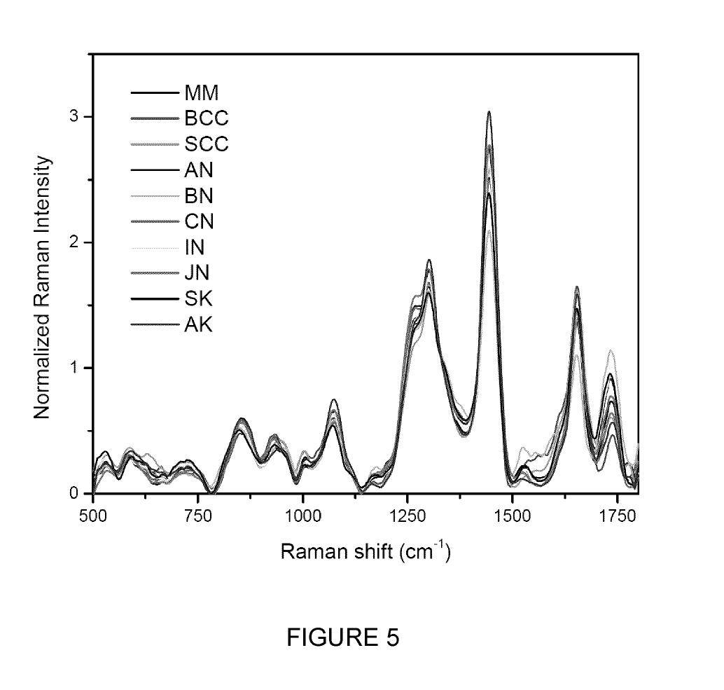

The mean Raman spectra for different skin pathologies for the lesions of Table I are depicted in FIG. 5. All spectra were normalized to their respective areas under curve (AUC) before being averaged in aggregate for each diagnosis. Overall the Raman spectra for the skin lesions included in this study share similar major Raman peaks and bands. The strongest Raman peak is located around 1445 cm⁻¹ with other major Raman bands centered at 855, 936, 1002, 1271, 1302, 1655 and 1745 cm⁻¹. Non-pigmented skin lesions such as BCC, SCC and AK, have relatively lower relative intensities around 1745 cm⁻¹ than those of pigmented skin lesions (MM, AN, BN, CN, IN, JN, SK). When the 1745 cm⁻¹ peak is compared in a pairwise manner between pigmented lesions and their corresponding surrounding normal skin the same trend is apparent (i.e. the intensity around 1745 cm⁻¹ is higher for the pigmented lesion than for the corresponding normal tissue). The peak intensity around 1655 cm⁻¹ is much higher for non-pigmented lesions (BCC, SCC, AK) than pigmented lesions (MM, AN, BN, CN, IN, JN, SK). A peak at approximately 1520 cm⁻¹ appears to be closely related to melanin. The Raman intensity around 1370 cm⁻¹ for pigmented lesions (MM, AN, BN, CN, IN, JN, SK) is seen to be higher than that of non-pigmented lesions (BCC, SCC, AK).

Statistical methods may be applied to extract the diagnostic information that is intrinsically embedded within data-rich Raman spectra.

One factor that can influence the reliability of tissue characterization and/or tissue differentiation is the spectral range within the Raman spectrum that is used. It has been found that it is better to use a full spectral range (including 500 cm⁻¹ to 1800 cm⁻¹ or more) for differentiation of cancer and actinic kereosis from benign lesions. It has been found that a more restricted range (e.g. 1055 cm⁻¹ to 1800 cm⁻¹) can provide better differentiation of melanoma from non-melanoma pigmented lesions and for differentiation of melanoma from sebhorreic keratosis. In some embodiments a spectrum analysis component is configured to derive a plurality of indicators for a plurality of different differentiations based on a plurality of different ranges within the Raman spectrum. Spectra may be normalized (for example by normalizing the area under curve (AUC) within each of the ranges). In some embodiments the ranges to be used are selected based upon the variances in Raman signal intensities for multiple spectra acquired for tissues having the same pathology.

To assess the repeatability of Raman spectra, a study was performed which involved taking multiple spectra from the same sites. The study involved 15 different skin lesions and 15 different normal skin locations. The Raman frequency shifts by wavenumber (abscissa) for any three consecutive spectra from the same tissue site were found to vary only negligibly. However, the intensity of the Raman signal was found to vary significantly and the variances for the triplicate measurement sets demonstrated a systematic change that was wavenumber-dependent. The spectra tended to have a smoother portion (in which the spectra varied less) at lower Raman shifts and to fluctuate more at higher Raman shifts. At 1055 cm⁻¹ the smoother portion of the spectra reached a minimum variance for nearly all the repeat-matched spectral measurements. Based on this analysis of Raman measurement reproducibility, the skin spectra were analyzed not only using the full spectrum (500-1800 cm⁻¹), but also by separately considering only low (500-1055 cm⁻¹) and high (1055-1800 cm⁻¹) ranges.

In one example embodiment spectrum analysis component 42 is configured to determine a first indicator for differentiating one or more of cancer and actinic kereosis from benign lesions using a first range within the Raman spectrum and to determine a second indicator for differentiating melanoma from non-melanoma pigmented lesions and/or melanoma from sebhorreic keratosis using a second range within the Raman spectrum that is smaller than the first range. In some embodiments the second range overlaps with the first range by at least 90% or 95% of the second range. In some embodiments the second range is completely within the first range. In one example embodiment the first range is about 500 cm⁻¹ to 1800 cm⁻¹ and the second range is about 1055 cm⁻¹ to 1800 cm⁻¹. As described above, the choice of 1055 cm⁻¹ as one end of the second range is not arbitrary. However, it is not mandatory that the second range start at exactly at 1055 cm⁻¹. Significant variation in this end of the second range is possible. For example, the second range could begin at Raman shifts of 1055 cm⁻¹±40 cm⁻¹ or 1055 cm⁻¹±15 cm⁻¹.

It is not necessary that indicators be based on the entire range of Raman spectral data. In some cases, an analysis unit is configured to determine indicators for classification and/or discrimination based on a plurality of sub-ranges within the range. This can improve reliability in the results by emphasizing those sub-ranges which differ most between different pathologies that it is desired to discriminate between.

Some example ways to apply measurements of Raman spectra in selected bands are: to perform principal components analysis and discriminant analysis based upon the Raman spectra in those sub-ranges; to perform linear discriminant analysis based directly on integrated intensities of the Raman spectra in the sub-ranges; and/or to perform measurements on specific features within the sub-ranges.

For example, it has been found that there are significant differences between Raman spectra for tissues affected by cancer or actinic kereosis, on the one hand, and benign lesions, on the other hand, in the following 14 sub-ranges: 500-513, 546-586, 611-675, 721-736, 760-830, 870-900, 947-1320, 1345-1420, 1434-1457, 1478-1520, and 1540-1790 cm⁻¹. There are significant differences between Raman spectra for tissues affected by cancer, on one hand, and benign lesions, on the other hand, in the following sub-ranges 500-511, 546-584, 618-675, 721-1210, 1232-1288, 1351-1422, 1468-1500, 1533-1681, and 1693-1800 cm⁻¹. There are significant differences between Raman spectra for tissues affected by malignant melanoma, on one hand, and other pigmented lesions, on the other hand, in the following sub-ranges: 1055-1100, 1292-1322, 1357-1414, 1426-1480, 1617-1644, 1672-1721, and 1769-1787 cm⁻¹. There are significant differences between Raman spectra for tissues affected by malignant melanoma, on one hand, and seborreic keratosis, on the other hand, in the following sub-ranges: 1055-1106, 1143-1147, 1255-1263, 1288-1322, 1343-1416, 1428-1497, 1591-1649, 1665-1736, and 1760-1791 cm⁻¹.

In some embodiments a spectral analysis component 42 is configured to generate indicators indicating discriminations between different pairs of tissue pathologies or different pairs of groups of tissue pathologies using different sets of sub-ranges within the Raman spectrum. For example, a spectral analysis component may generate indicators for two or more of the pairs of tissue pathologies and groups of tissue pathologies listed above using the different sets of sub-ranges listed above.

In some embodiments, methods and apparatus are arranged to generate indicators for a plurality of different discriminations. The apparatus may be constructed to automatically generate the plurality of indicators or to allow a user to select one or more indicators to be generated by providing user input to a suitable user interface. For example, the plurality of indicators may comprise indicators relating to two or more of the following discriminations: a) discrimination between cancer or actinic kereosis, on the one hand, and benign lesions, on the other hand; b) discrimination between malignant melanoma, on one hand, and other pigmented lesions, on the other hand; c) discrimination between malignant melanoma, on one hand, and seborreic keratosis, on the other hand. Each of the plurality of indicators may be based on a corresponding set of sub-ranges. The portions of the Raman spectra within the set of sub-ranges to be used for any discrimination may be normalized to the AUC over the sub-ranges to be used.

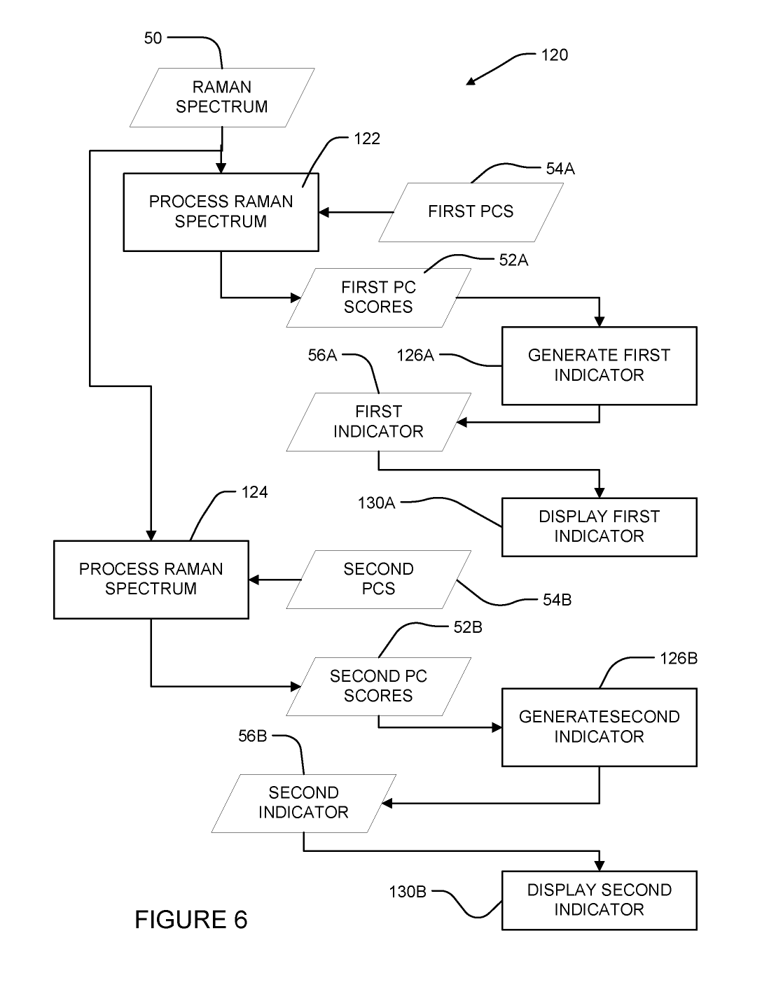

FIG. 6 shows an example spectral analysis method 120 configured to obtain indicators for two different discriminations based on a Raman spectrum 50. In block 122 the entire Raman spectrum 50 (or in some embodiments a portion of Raman spectrum 50) is processed to obtain first scores 52A corresponding to a first set of stored principal components 54A. In block 124 all (or in some embodiments a portion) of Raman spectrum 50 is processed to obtain second scores 52B corresponding to a second set of stored principal components 54B. In block 126A a first indicator 56A is determined from first scores 52A. In block 126B, a second indicator 56B is determined from second scores 52B. In block 130A, graphical or textual indicia of first indicator 56A is displayed. In block 130B, graphical or textual indicia of second indicator 56B is displayed. In an alternative embodiment, instead of principal components, one or both of blocks 122 and 124 is performed with partial least squares component weightings.

In some embodiments, blocks 122 and 124 process different ranges within the Raman spectrum. For example, block 122 may generate first PC scores 56A based upon a first range of the Raman spectrum and block 124 may generate second PC scores 56B based upon a second range of the Raman spectrum smaller than the first range. The ranges may overlap. In some embodiments, blocks 122 and/or 124 process predetermined sub-ranges within the Raman spectrum. Examples of such sub-ranges are described above.

Methods and apparatus as described herein may additionally be configured to discriminate between cancer sub-types. For example, as between superficial, nodular, pigmented and other forms of BCC.

The ability to discriminate between tissues affected by malignant melanoma, on one hand, and other pigmented lesions, on the other hand, is of particular value because many melanomas appear banal and may be overlooked, while many benign pigmented lesions appear malignant and unnecessarily biopsied. It is estimated that if all atypical pigmented lesions were to be biopsied to rule out melanoma, the biopsy ratio would be as high as 200:1, causing too many unnecessary biopsies.

Certain spectral peaks in the above ranges have been found to be particularly useful for discrimination between pathologies. Raman peaks that have particular utility for this purpose include the peaks at Raman shifts of 1370, 1520, 1570, 1655, and 1745 cm⁻¹. In some embodiments a spectrum analysis component is configured to search for and obtain measures one or more of these specific peaks and to base an indicator of discrimination or classification at least in part on the measure(s).

At least in cases where multivariate analysis is used as a tool for deriving indicators from Raman spectra it can be beneficial to use lesion spectra alone for discrimination and/or classification (i.e. without including spectra from surrounding normal skin in the analysis). In methods according to some embodiments Raman spectra of normal skin are not obtained or used.

Based on the purpose of diagnosis, different levels of sensitivity and specificity may be desired. In some embodiments, apparatus and methods as described herein permit user selection of modes which offer different combinations of sensitivity and specificity. Where a multivariate analysis such as PCA-GDA or PLS is performed to obtain an indicator for a discrimination between two pathologies or two sets of pathologies the sensitivity and specificity may be controlled by varying the discrimination function applied to generate the indicator. For example, the discrimination function may be selected to improve selectivity for one of the pathologies (or groups of pathologies) being discriminated between at the cost of some sensitivity or to improve sensitivity for the pathology or group of pathologies at the cost of some selectivity.

Apparatus according to some embodiments comprise a plurality of stored discrimination functions. One of the discrimination functions is selected based upon user input of an operating mode. In an example embodiment, apparatus is configured with a plurality of user-selectable modes. Each of the plurality of modes is operable to provide an indicator of a different discrimination. Each of the plurality of modes provides a different combination of sensitivity and selectivity. In some embodiments the apparatus provides two or more modes that provide indicators of the same discrimination in which the modes differ by providing different combinations of sensitivity and selectivity.

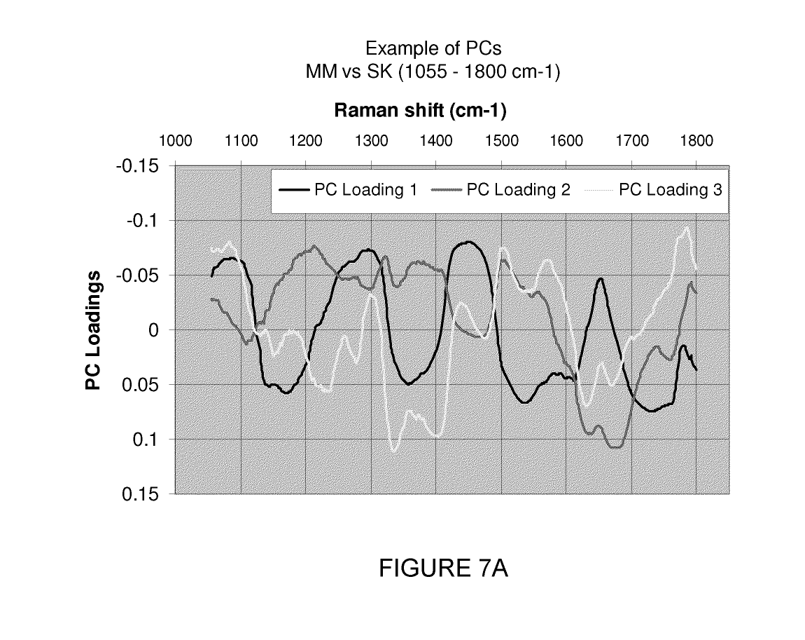

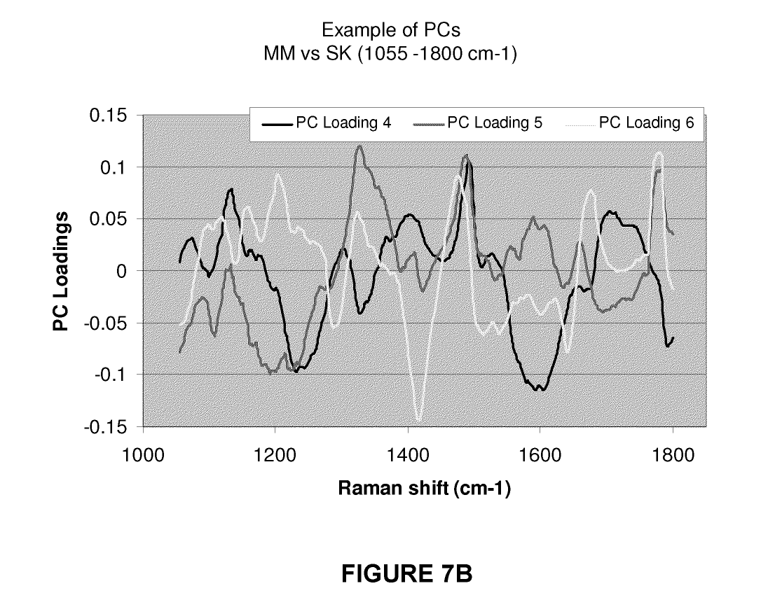

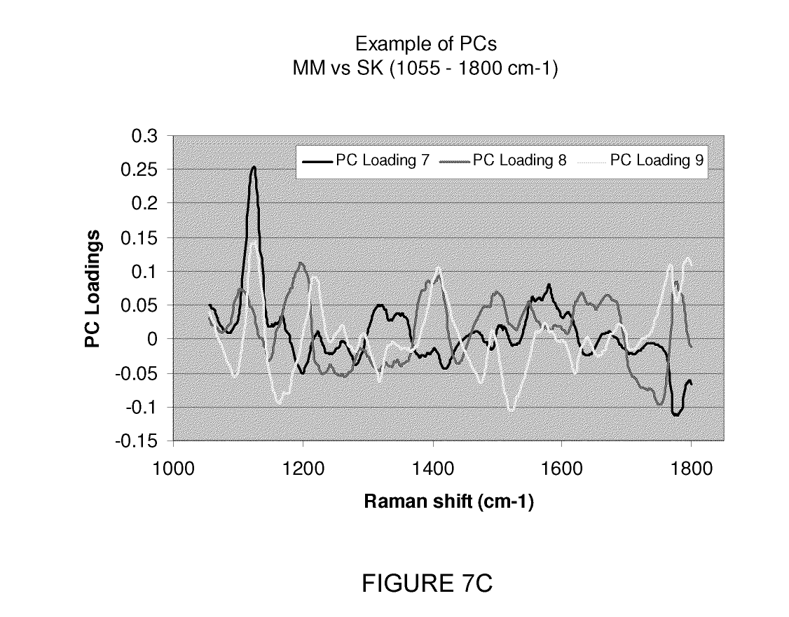

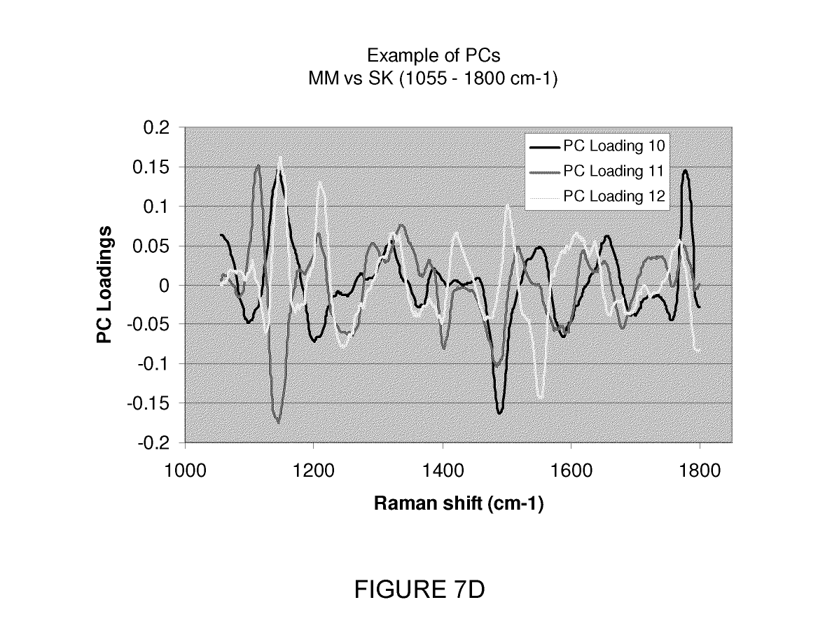

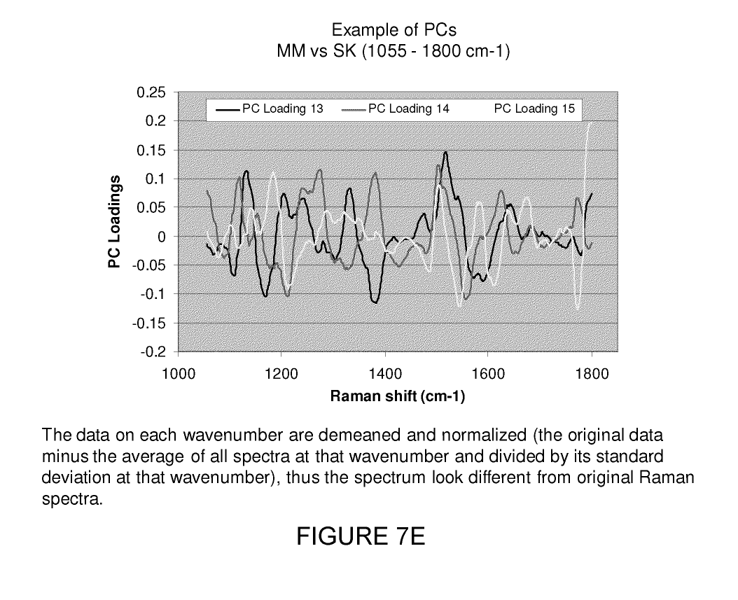

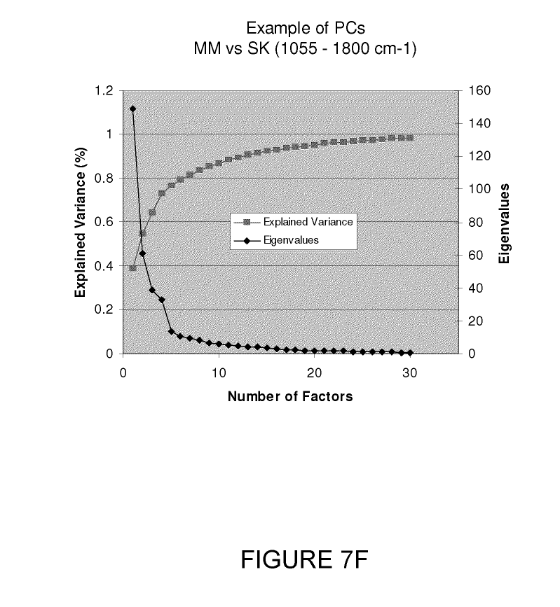

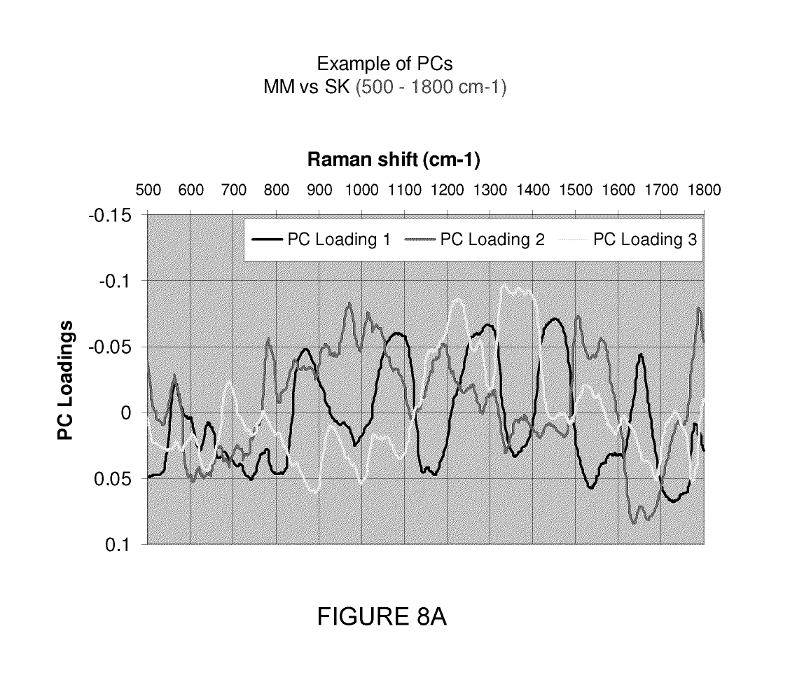

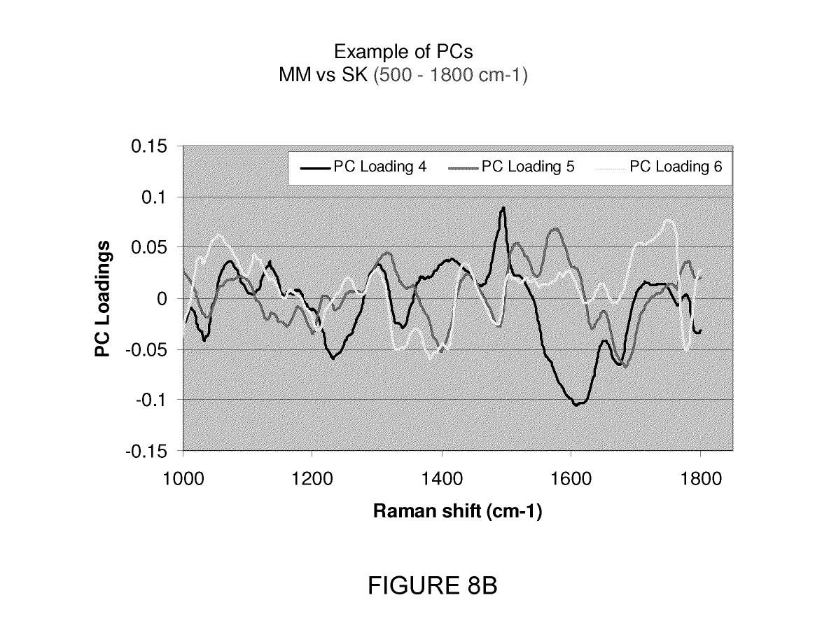

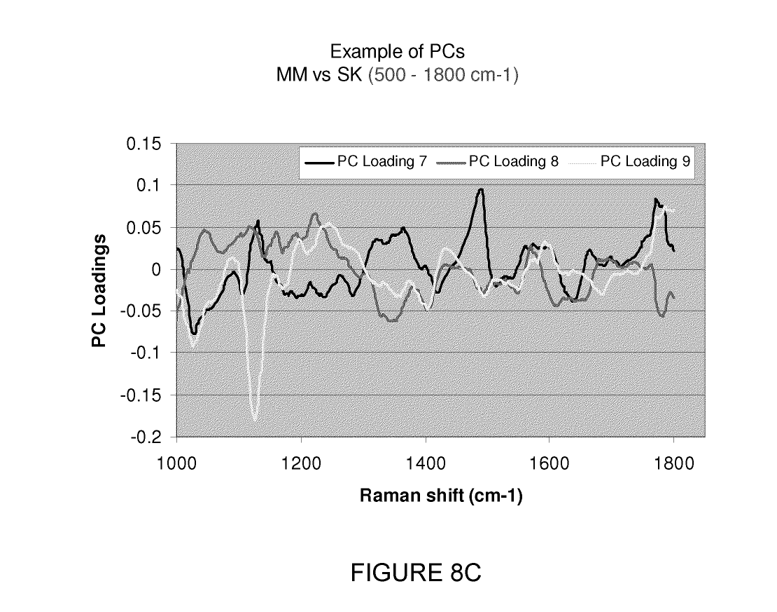

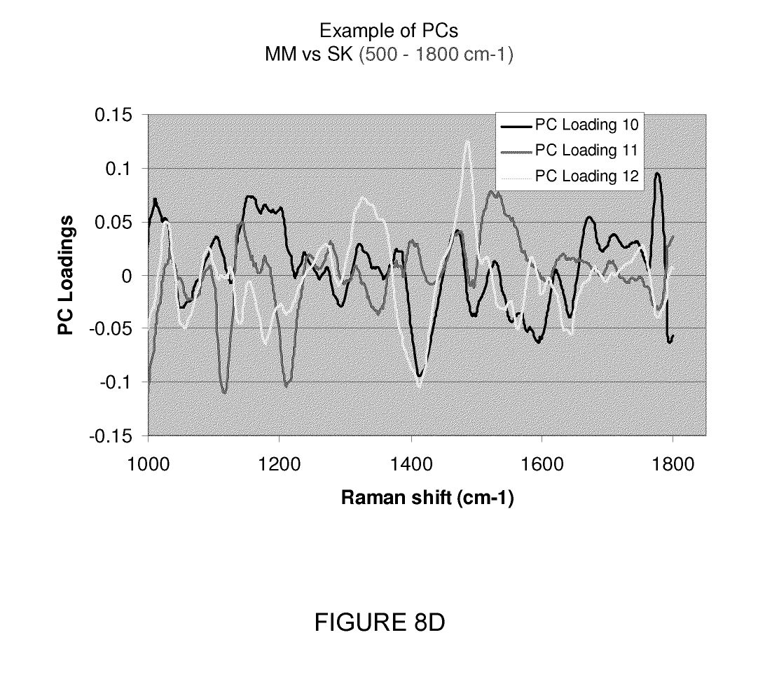

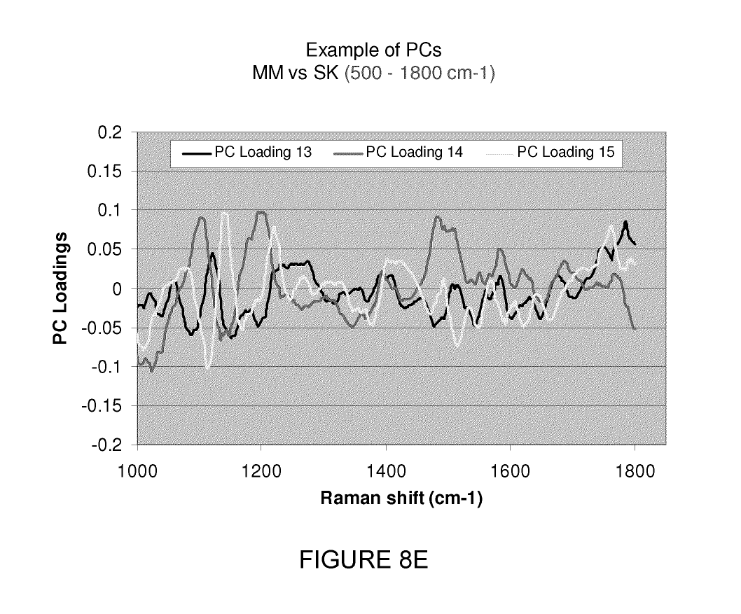

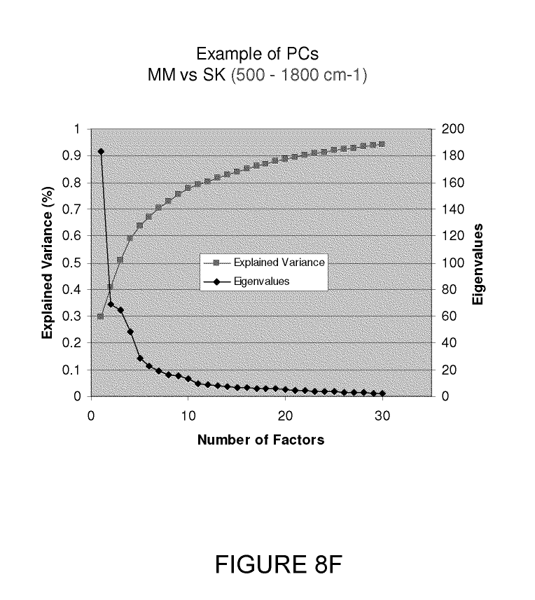

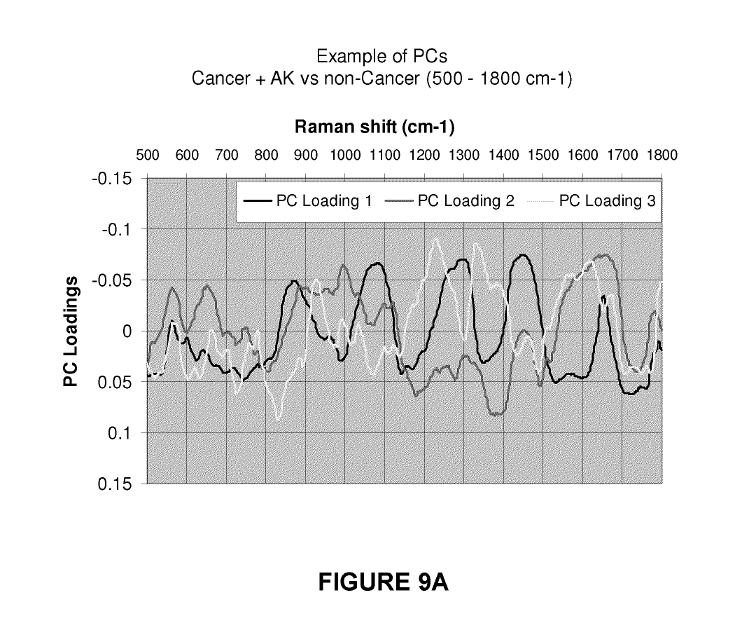

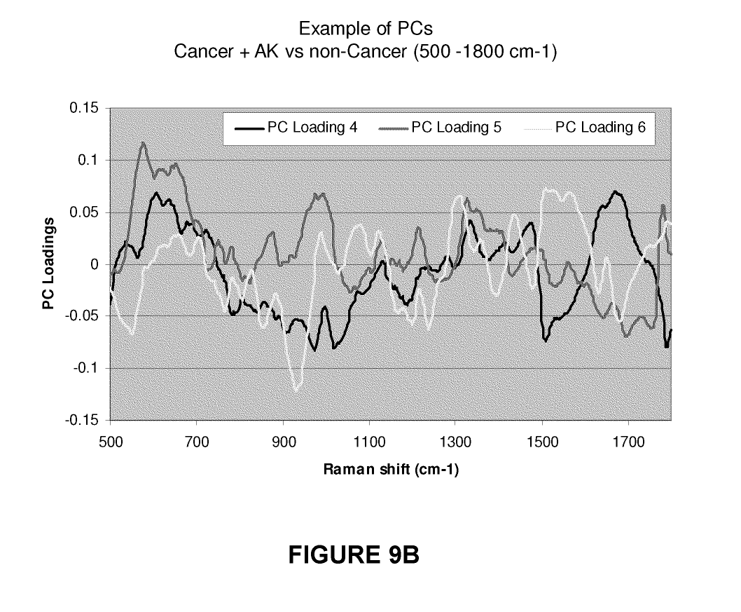

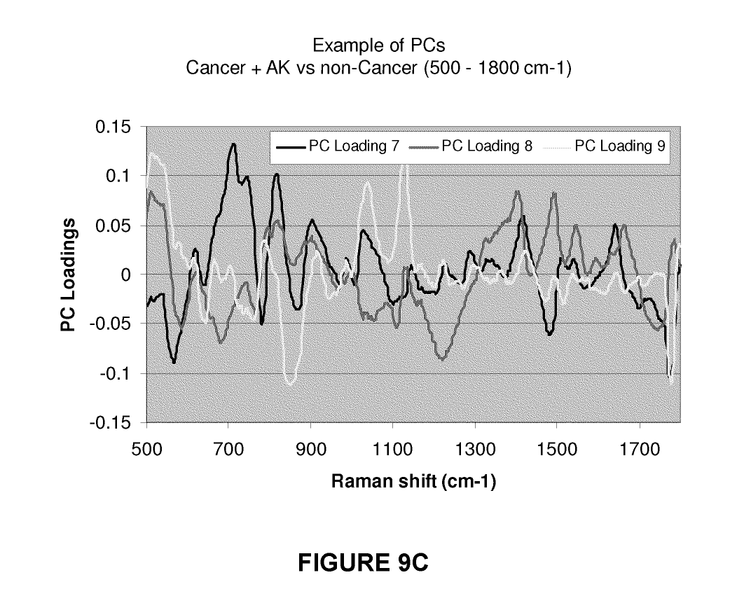

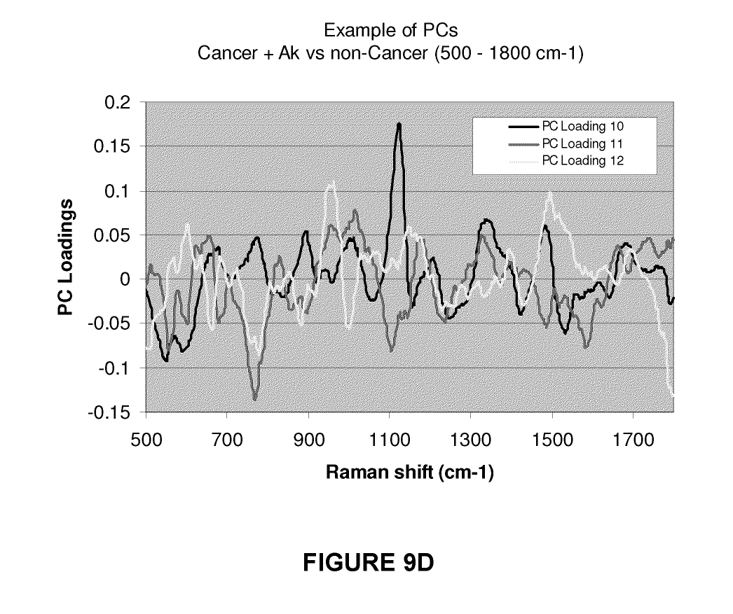

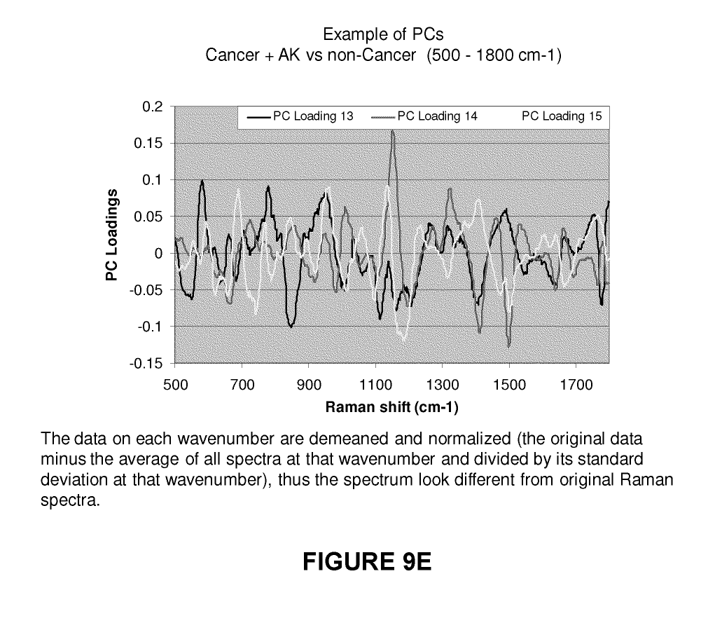

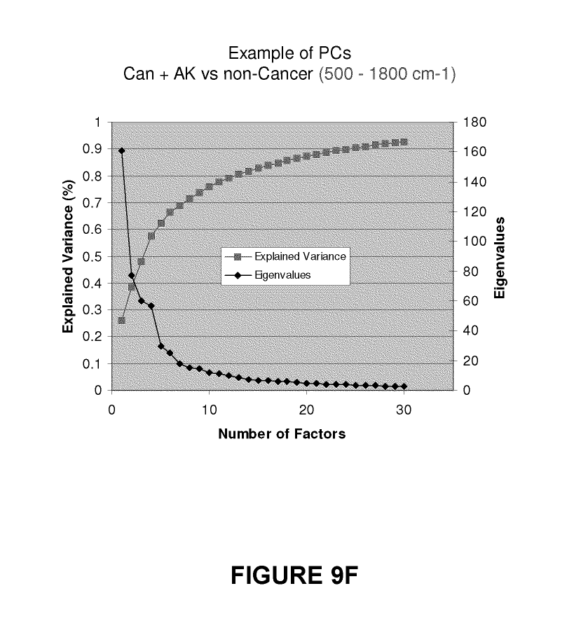

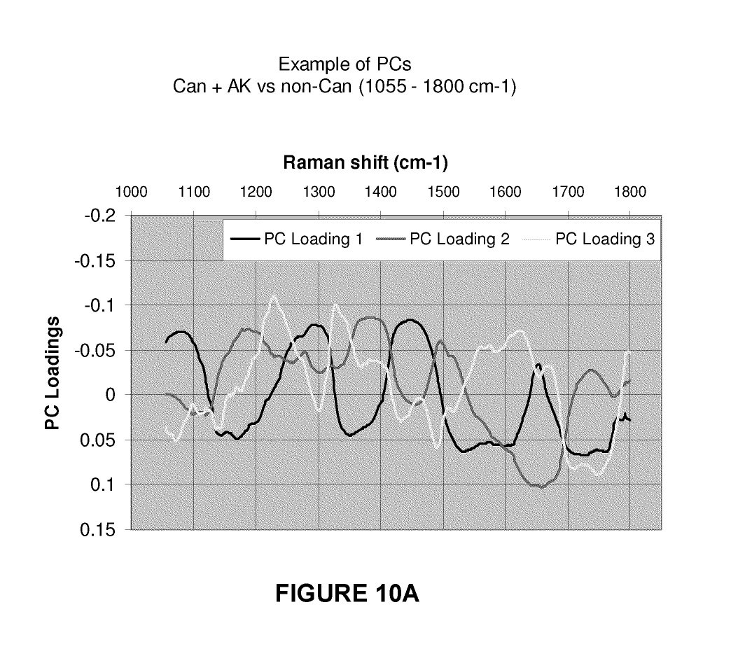

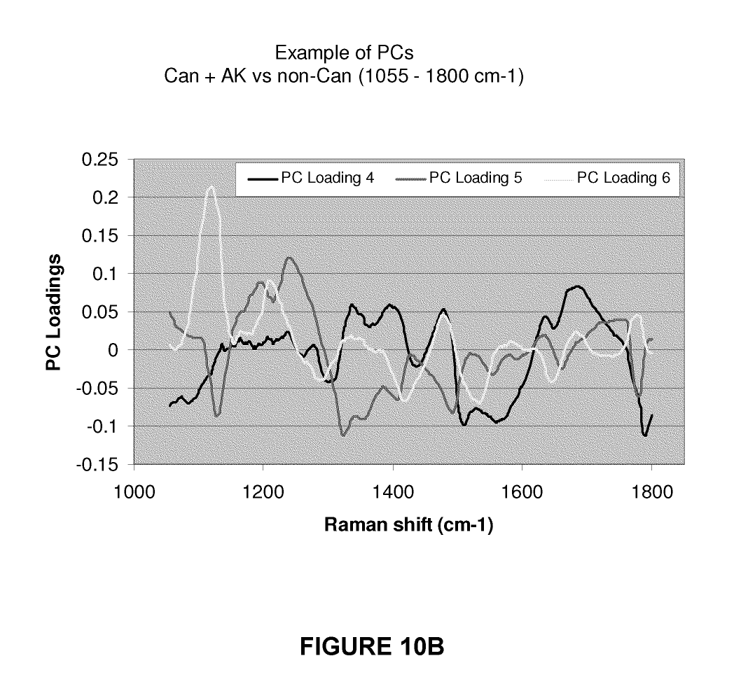

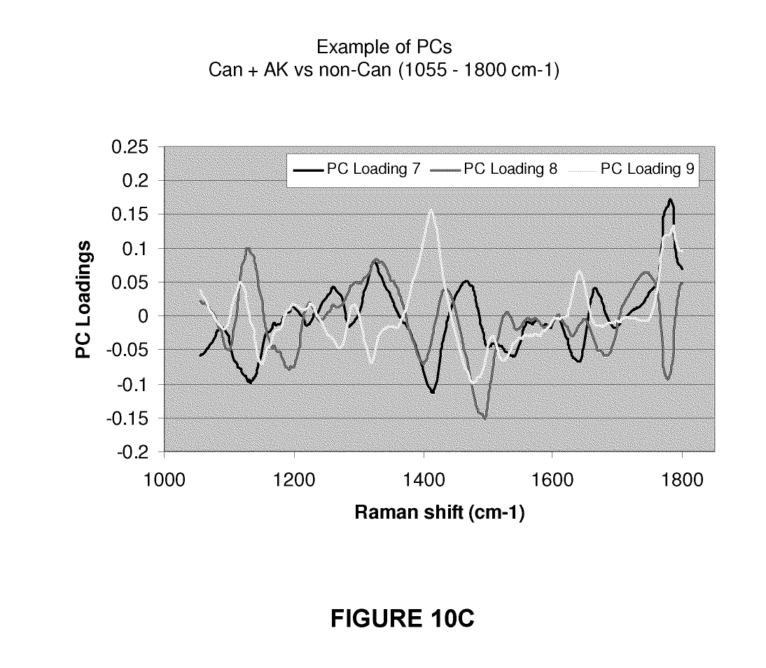

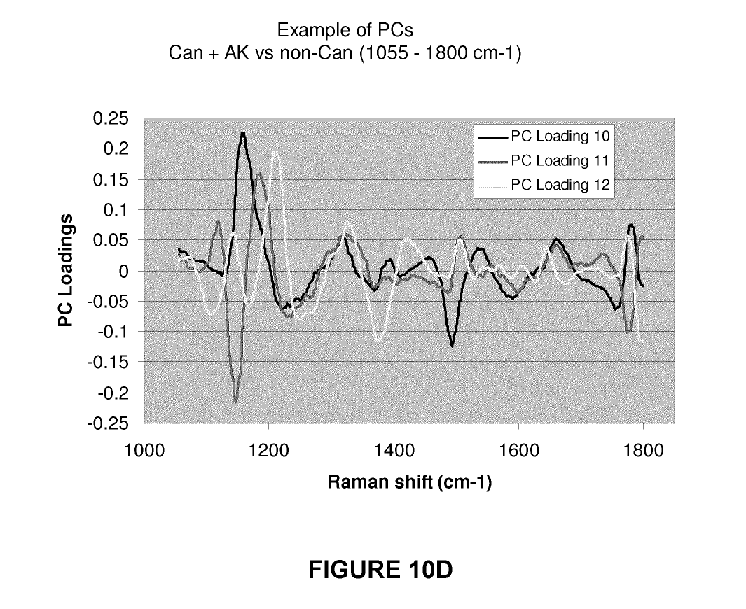

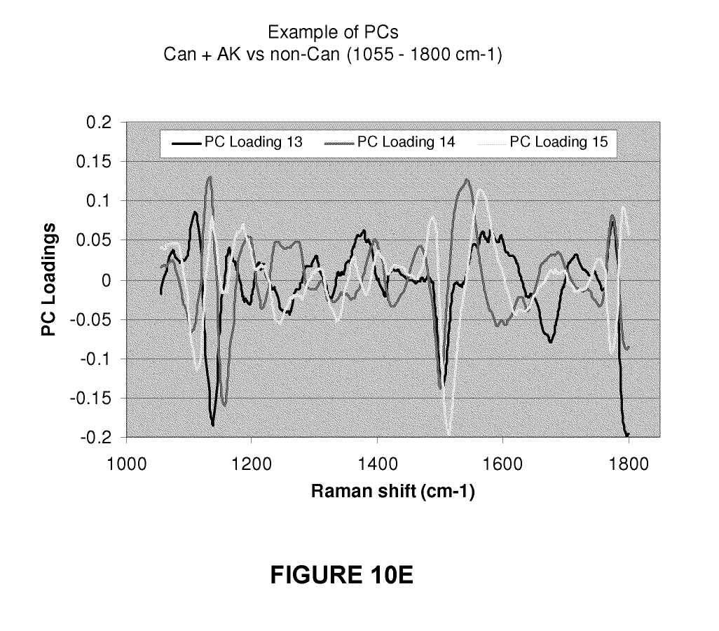

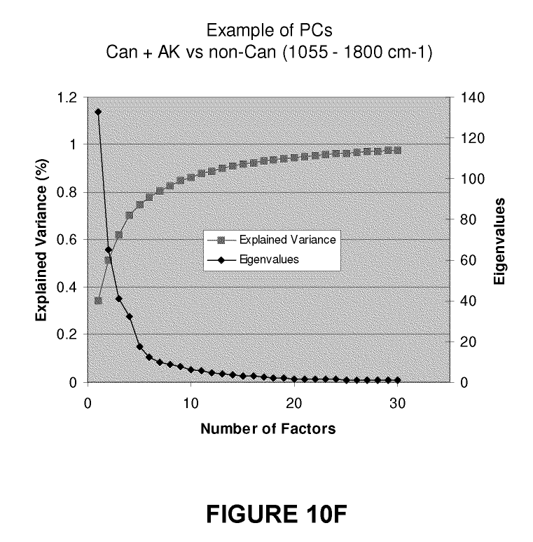

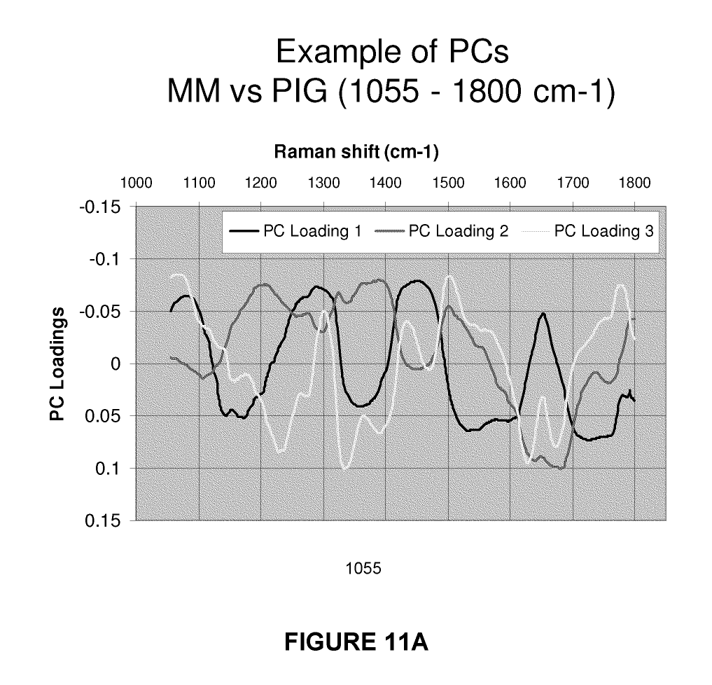

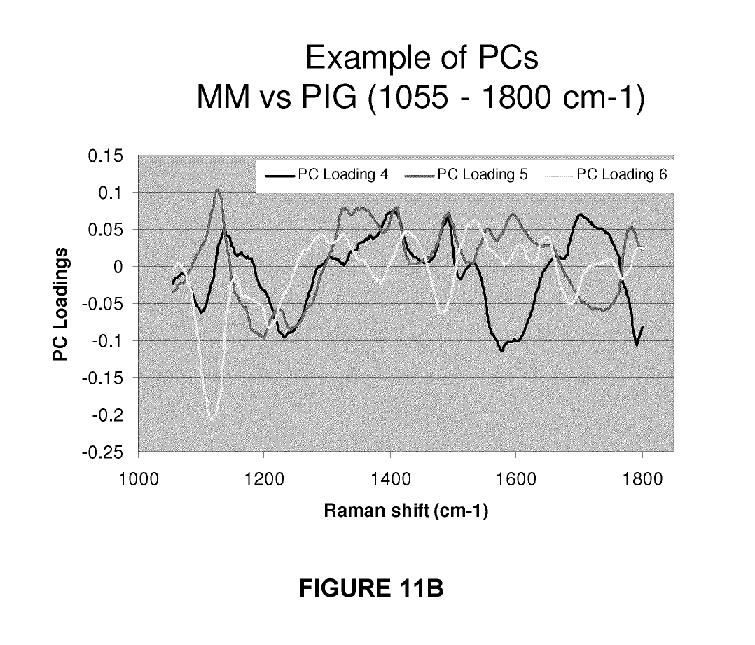

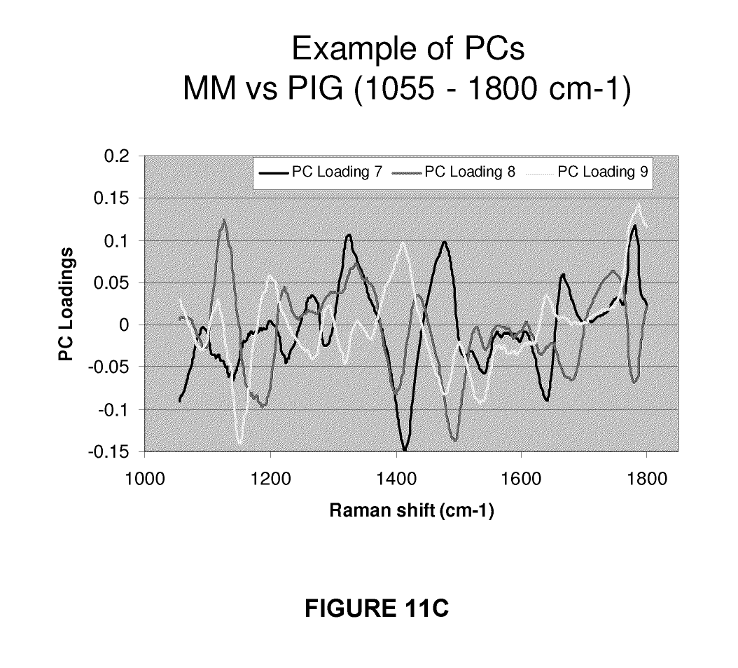

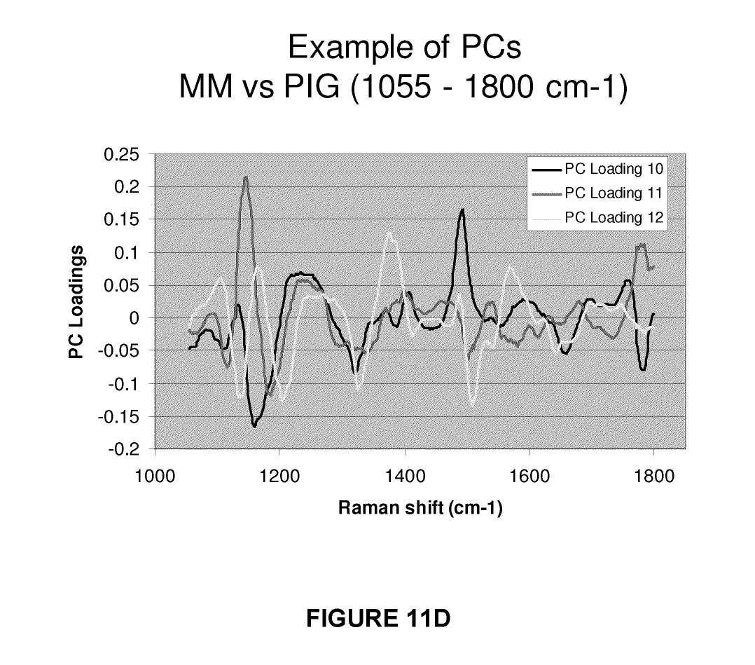

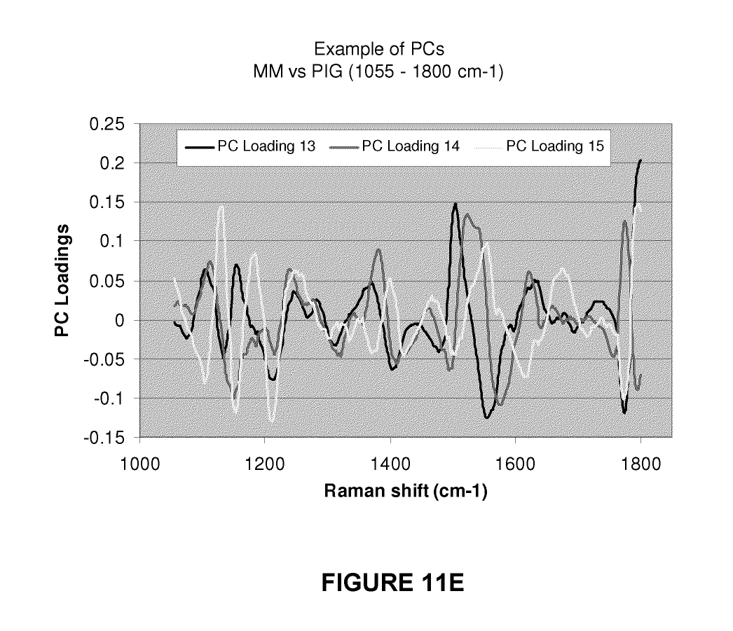

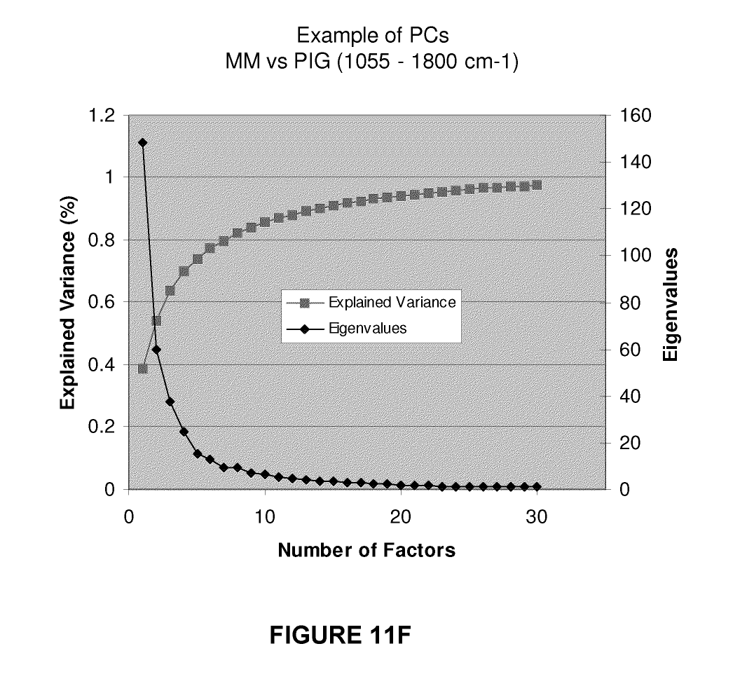

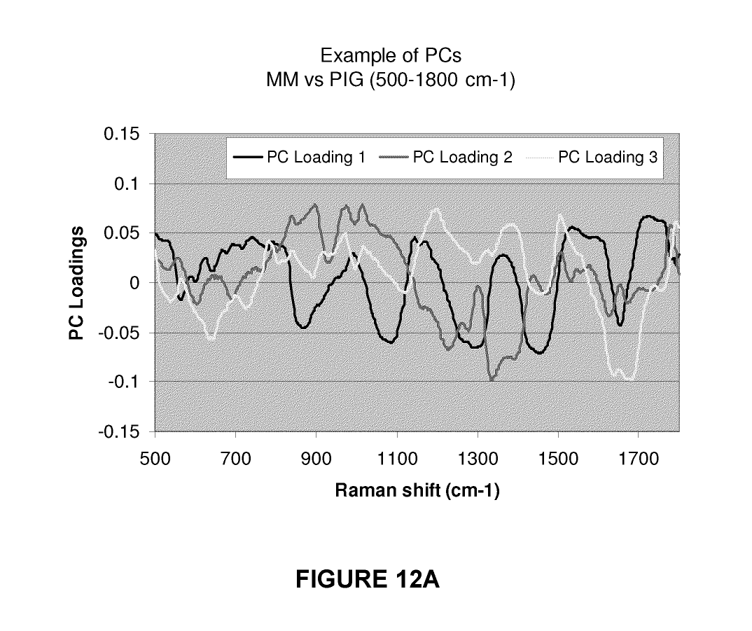

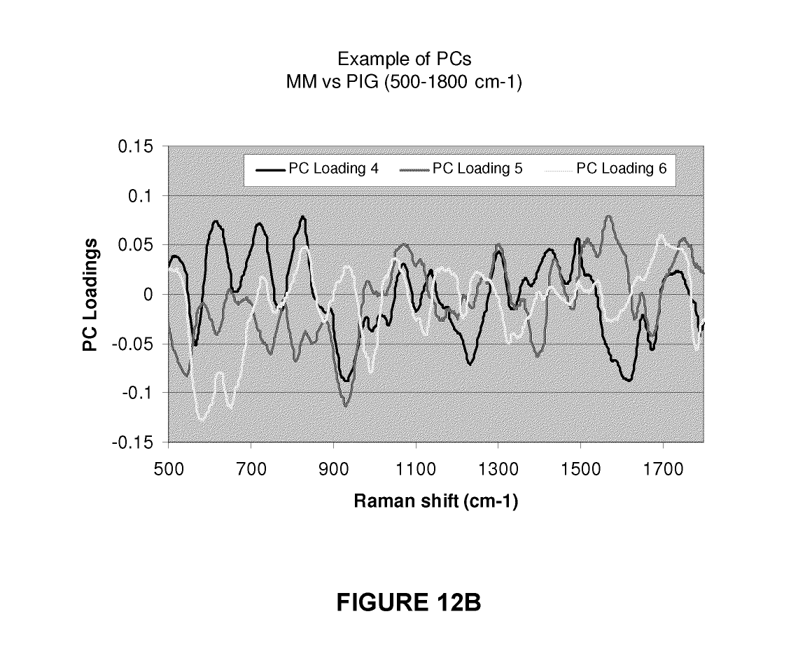

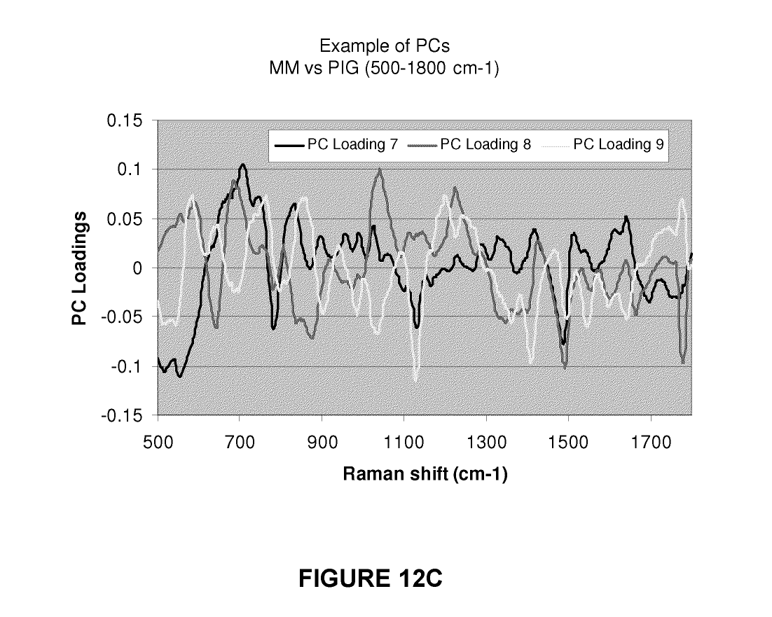

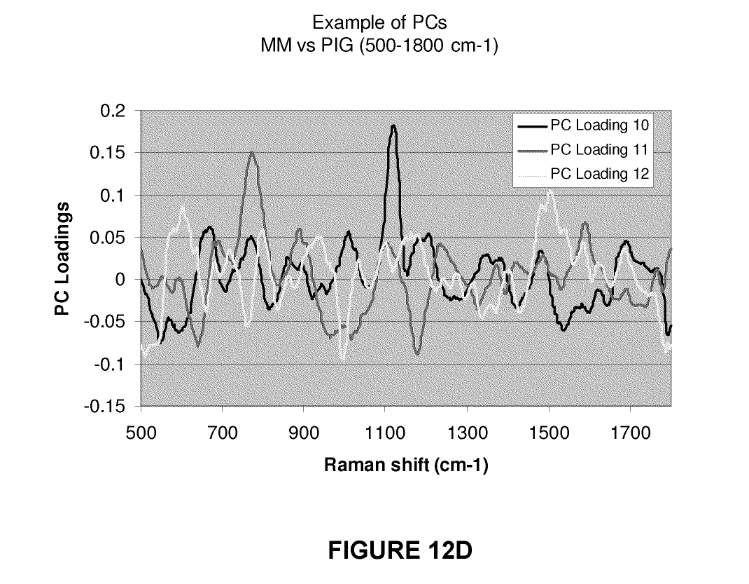

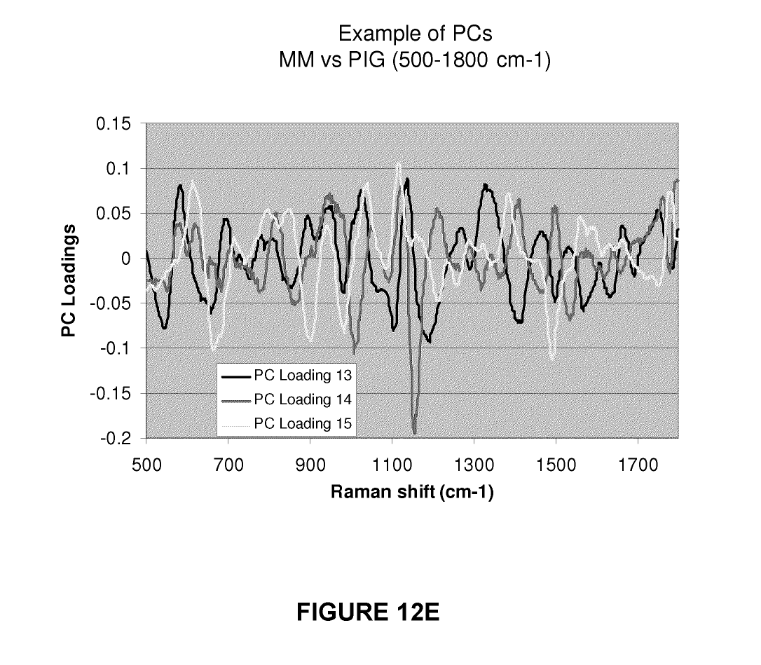

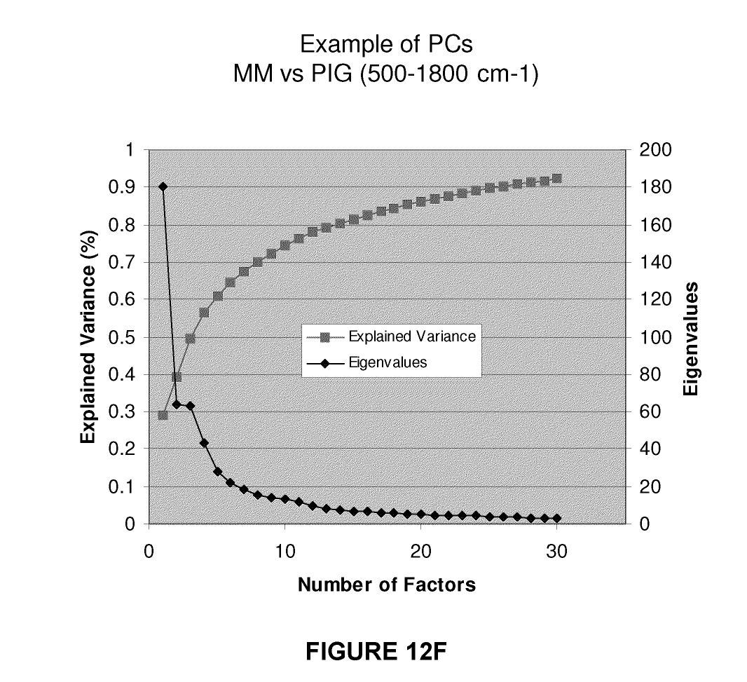

As noted above, the specific line-shapes of the principal components generated by a PCA analysis (PC1, PC2, PC3, . . . , PC15, . . . ) tend to pick up features in the Raman spectrum which distinguish different tissue pathologies. FIGS. 7A through 7E respectively show principal components generated for discriminating MM from SK using Raman spectra in the range of 1055 cm⁻¹ to 1800 cm⁻¹. FIG. 7F illustrates how the explained variances increases with the number of PC factors used. FIGS. 8A through 8E respectively show principal components generated for discriminating malignant melanoma (MM) from seborrheic keratosis (SK) using Raman spectra in the range of 500 cm⁻¹ to 1800 cm⁻¹. FIG. 8F illustrates how the explained variance increases with the number of PC factors used. FIGS. 9A through 9E show principal components generated for discriminating cancer and pre-cancer (AK) from non-cancer using Raman spectra in the range of 500 cm⁻¹ to 1800 cm⁻¹. FIG. 9F illustrates how the explained variances increases with the number of PC factors used. FIGS. 10A through 10E respectively show principal components generated for discriminating cancer and pre-cancer (AK) from non-cancer using Raman spectra in the range of 1055 cm⁻¹ to 1800 cm⁻¹. FIG. 10F illustrates how the explained variances increases with the number of PC factors used. FIGS. 11A to 11E show principal components generated for discriminating MM from non-melanoma pigmented lesions using Raman spectra in the range of 1055 cm⁻¹ to 1800 cm⁻¹. FIG. 11F illustrates how the explained variance increases with the number of principal components used. FIGS. 12A to 12E show principal components generated for discriminating MM from non-melanoma pigmented lesions using Raman spectra in the range of 500 cm⁻¹ to 1800 cm⁻¹. FIG. 12F illustrates how the explained variance increases with the number of principal components used.

The PCA analysis can optionally be made more efficient by selecting sub-ranges within the Raman spectrum that include spectral features that are particularly relevant to the tissue pathologies that are being discriminated between and/or classified. Some embodiments provide apparatus having a plurality of stored principal components that have features of one or more of the principal components illustrated in FIGS. 7A through 7E, 8A through 8E, 9A through 9E, 10A through 10E, 11A through 11E or 12A through 12E.

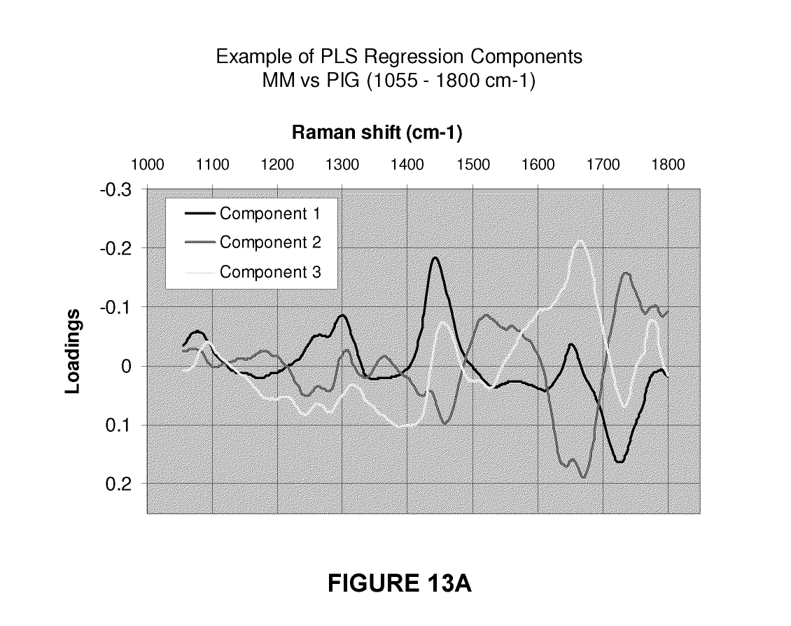

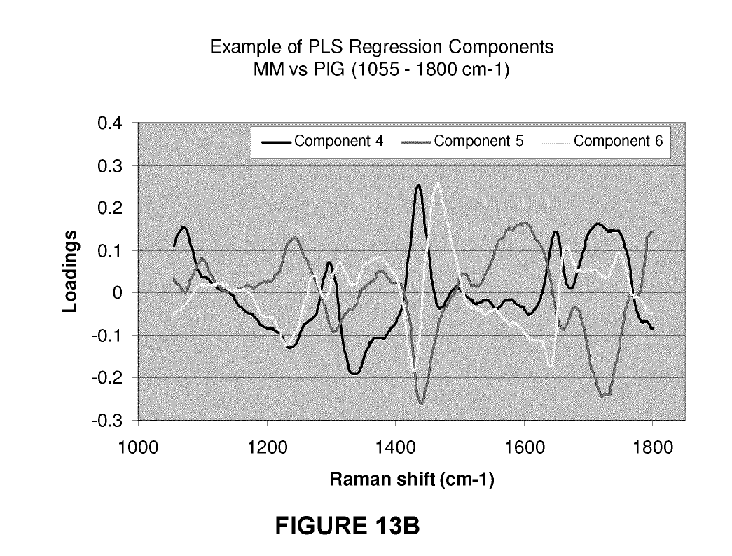

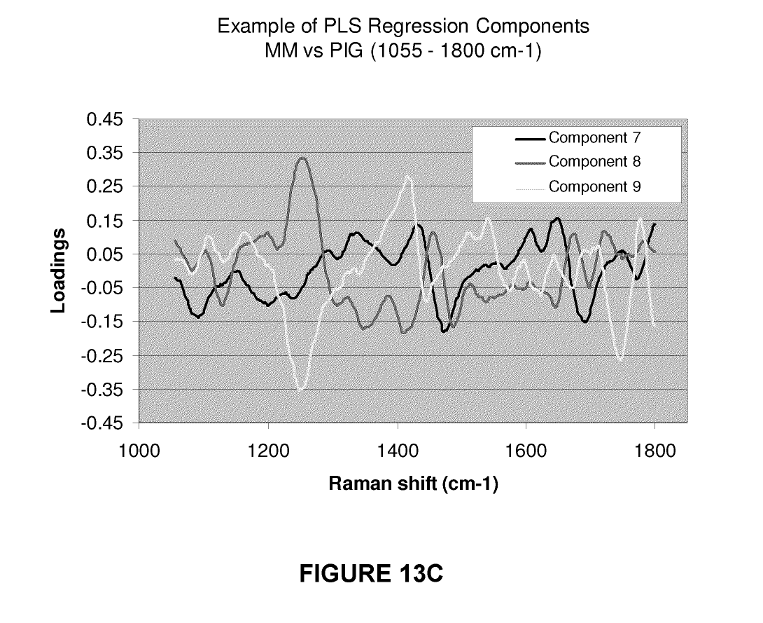

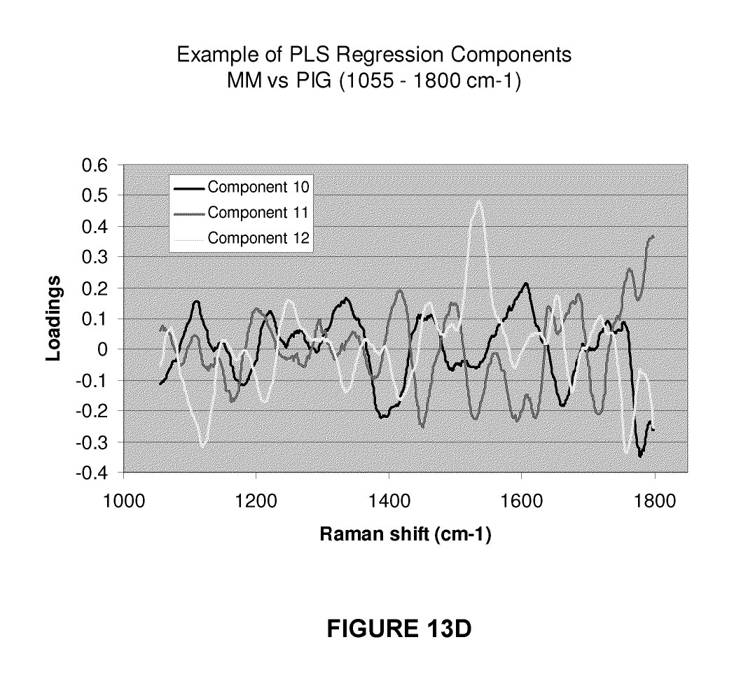

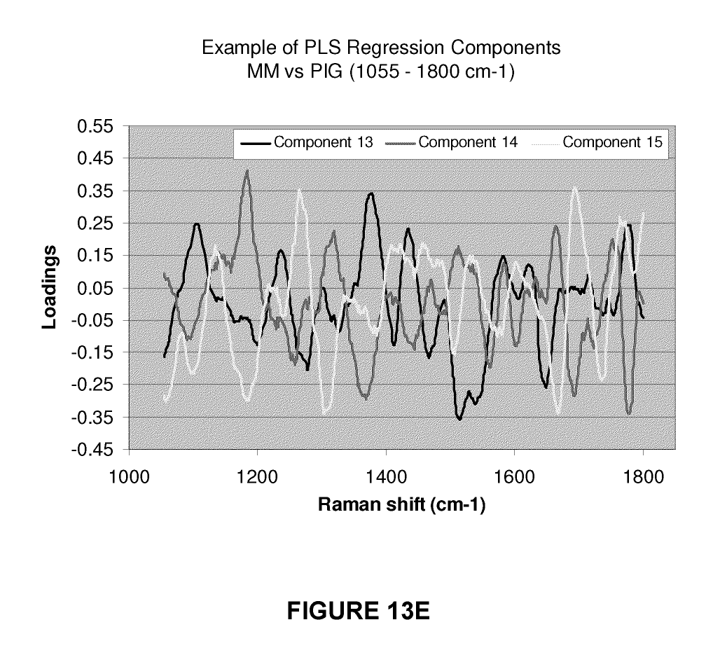

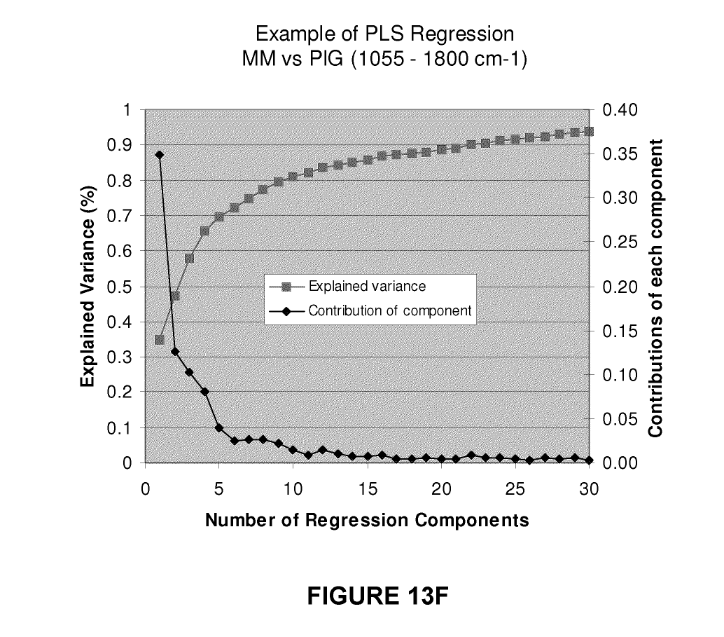

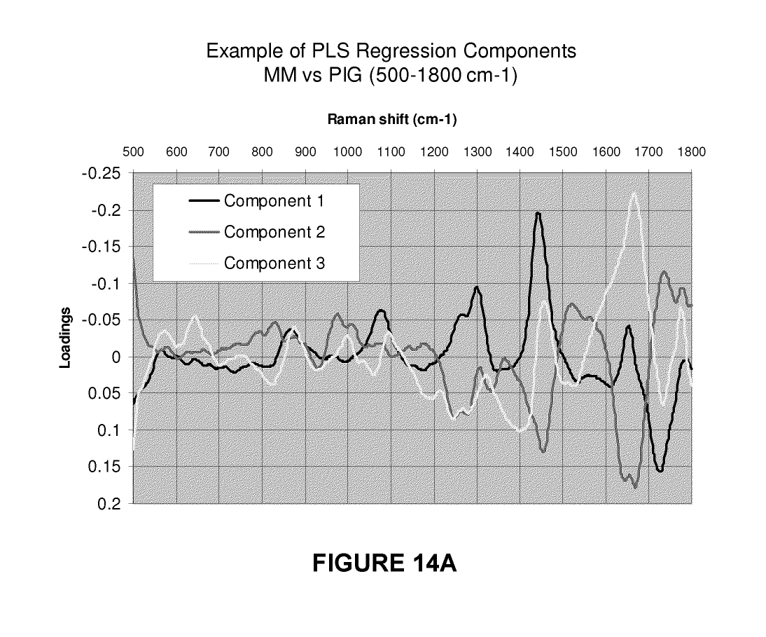

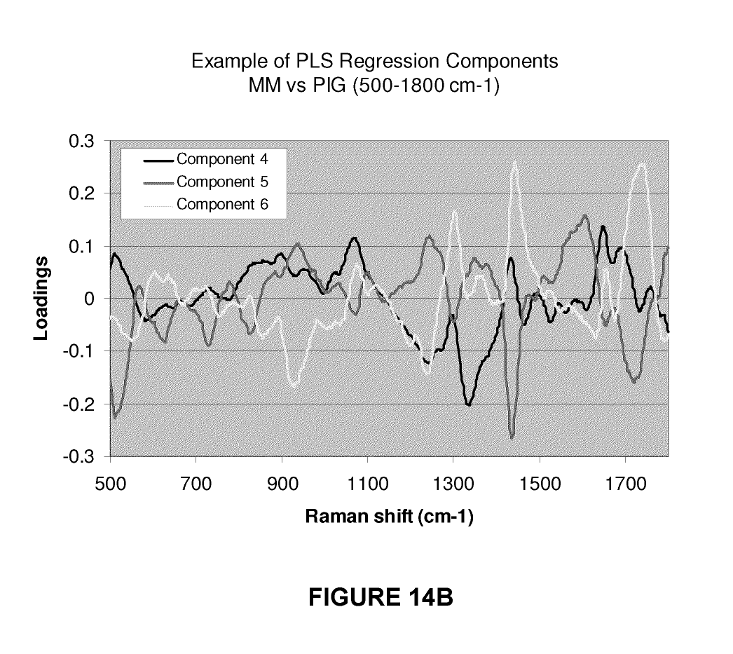

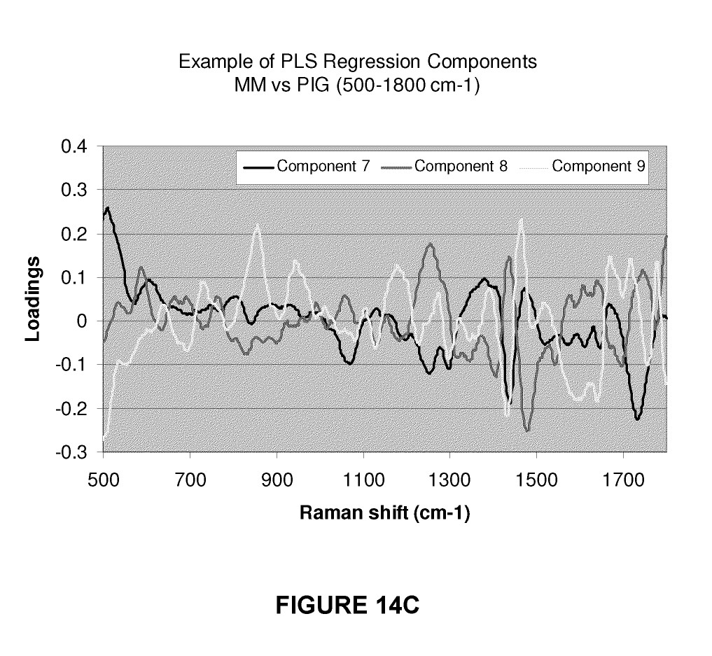

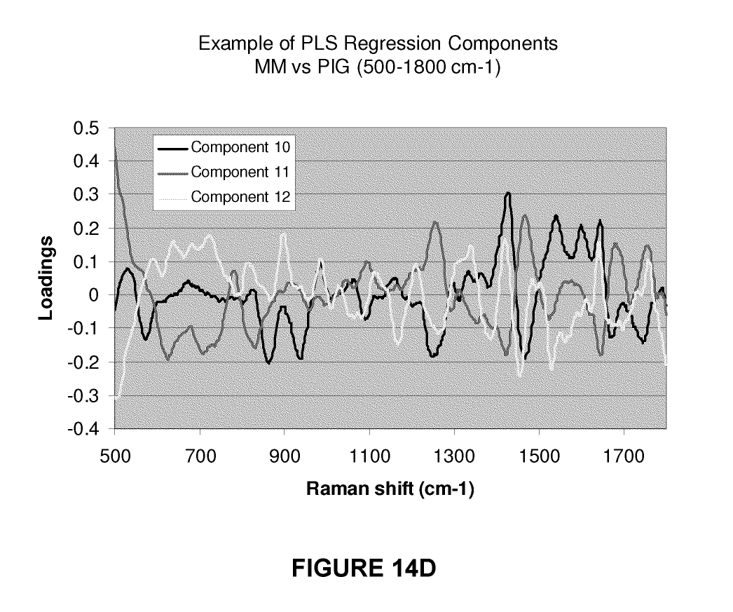

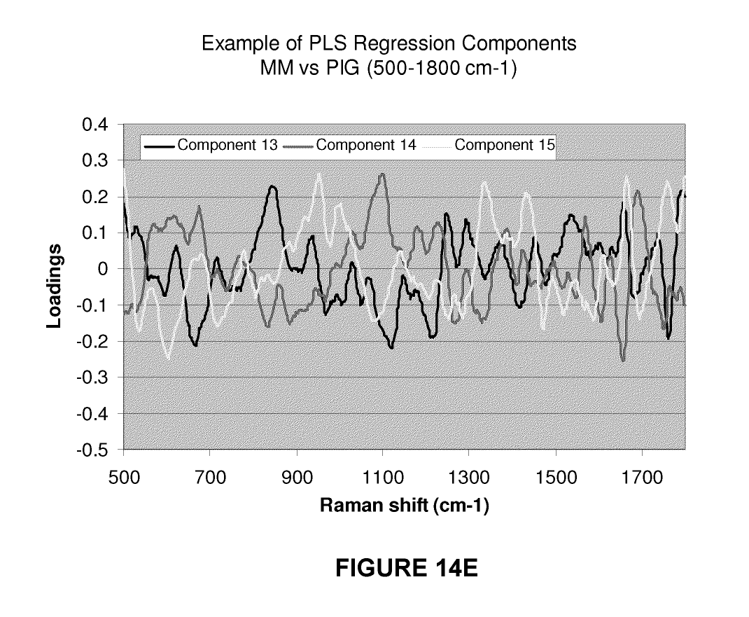

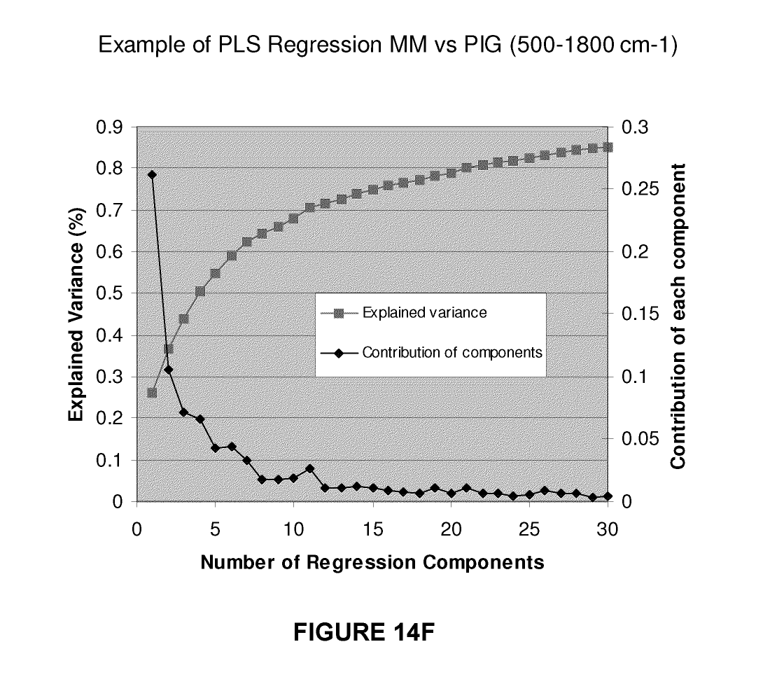

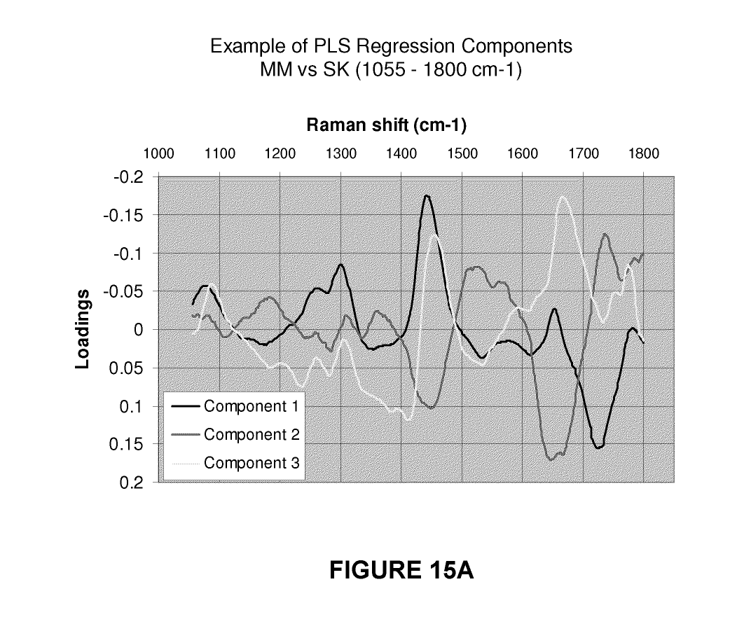

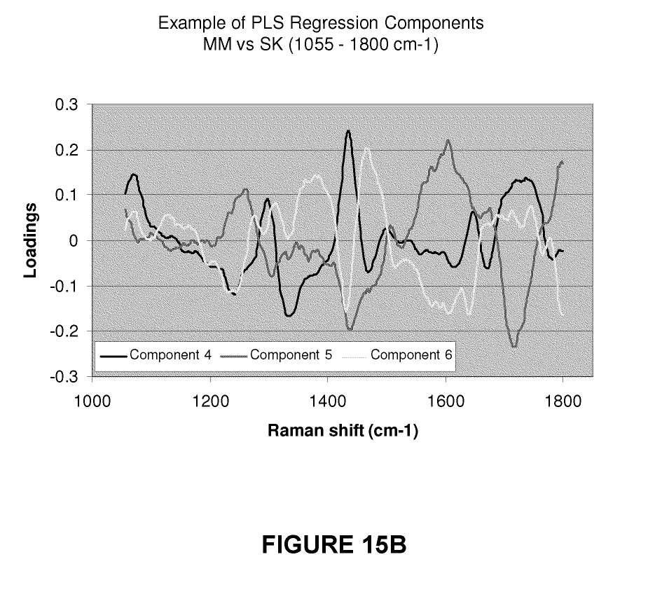

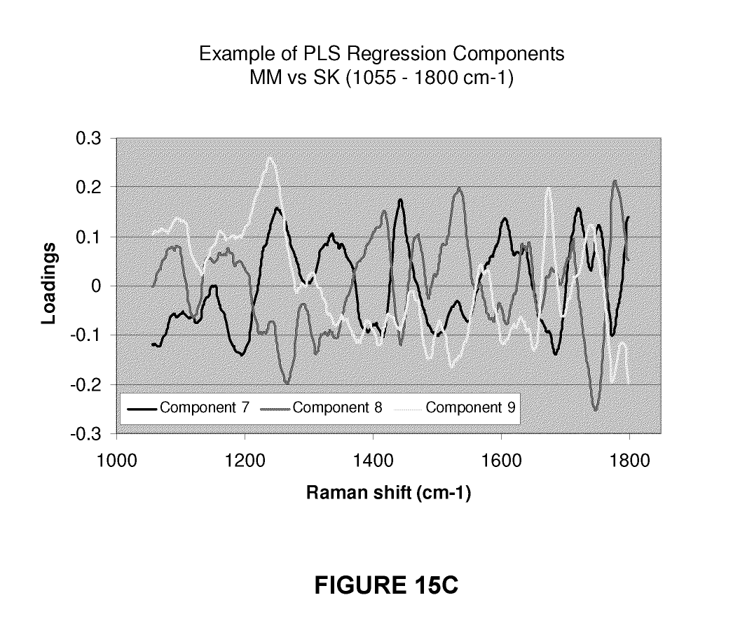

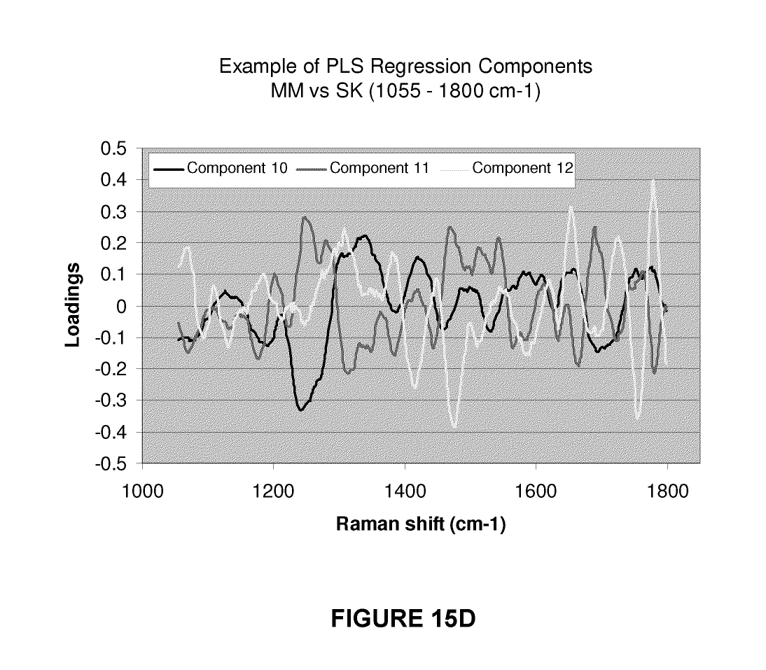

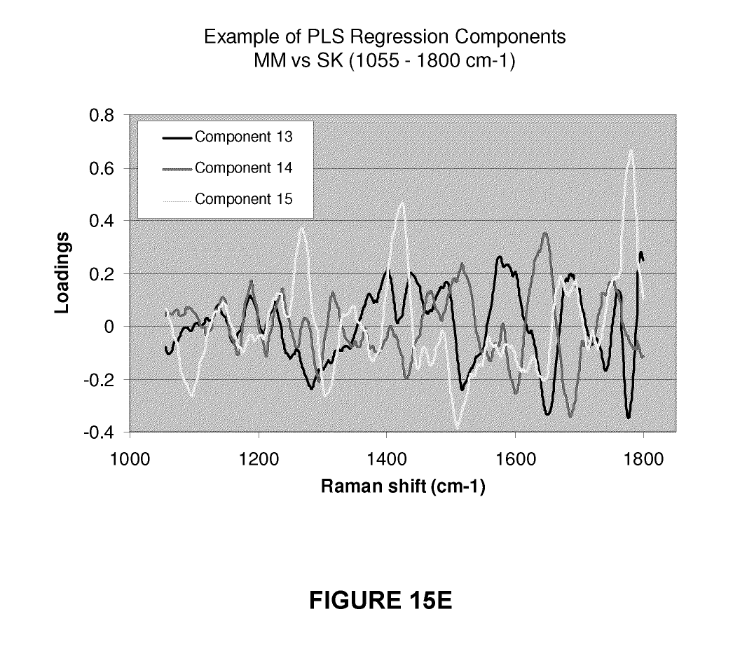

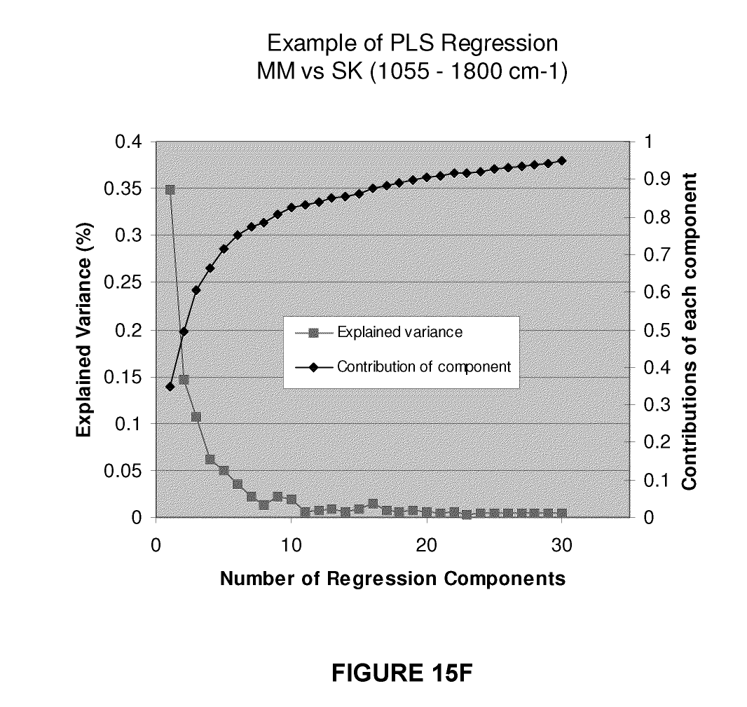

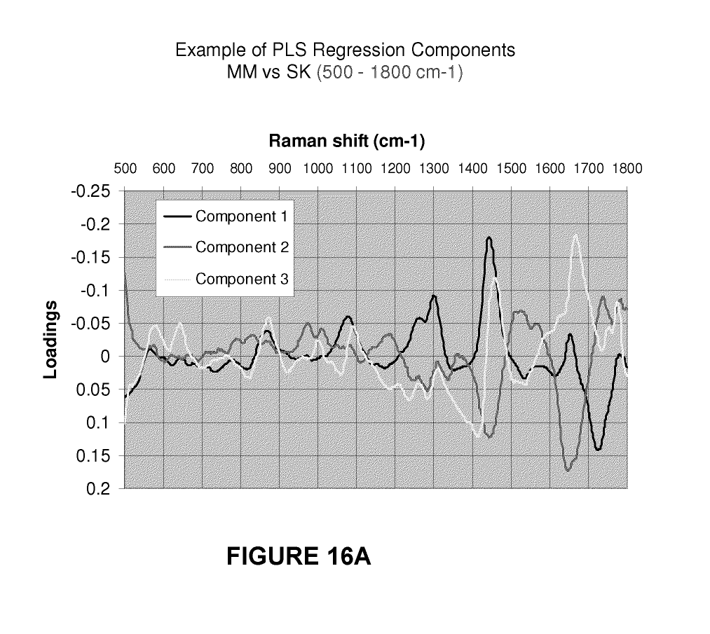

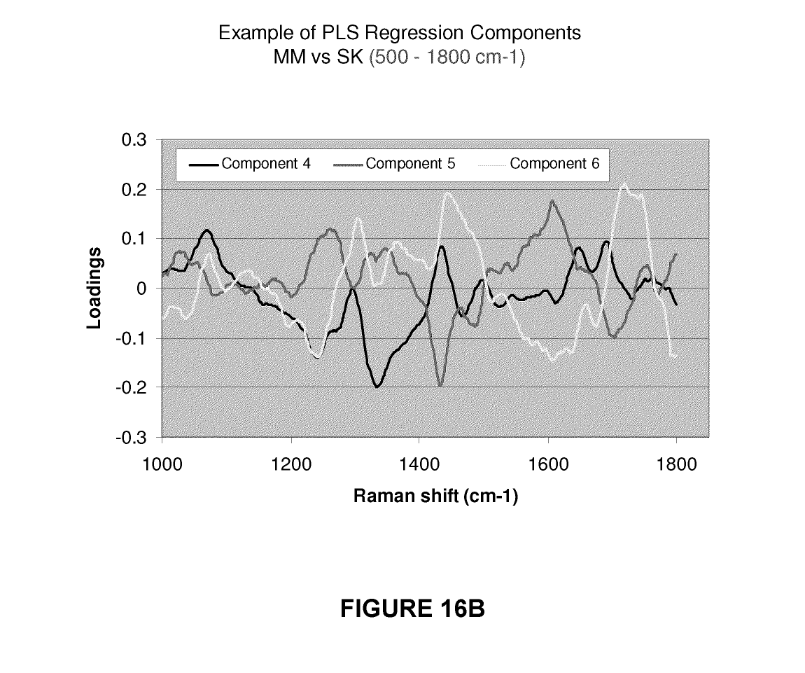

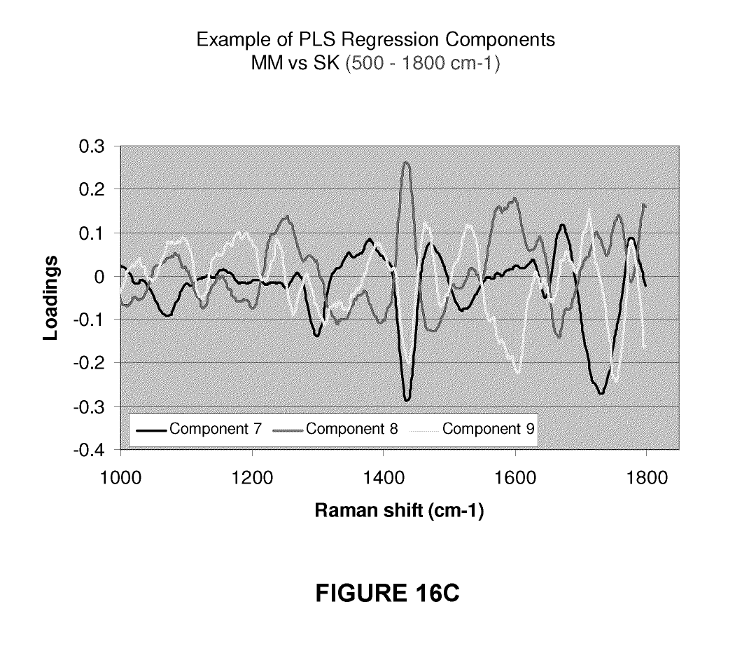

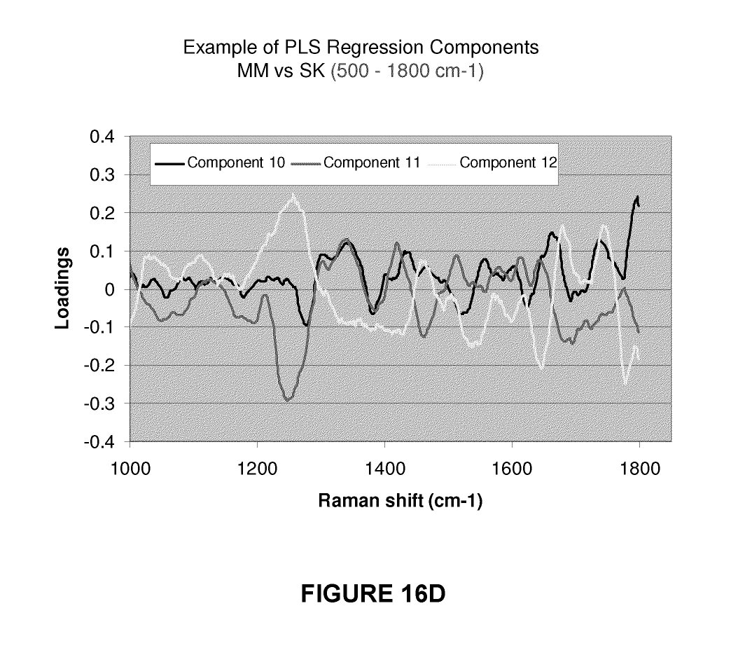

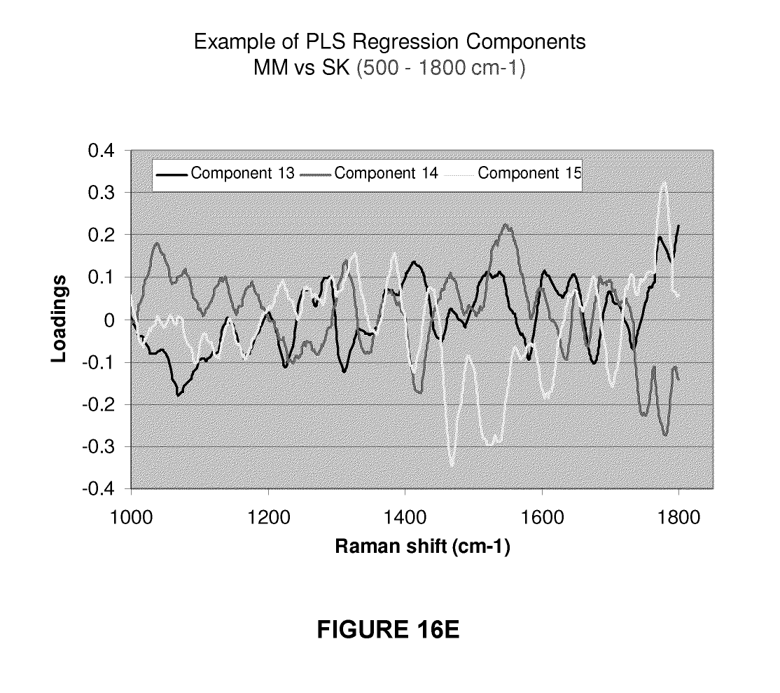

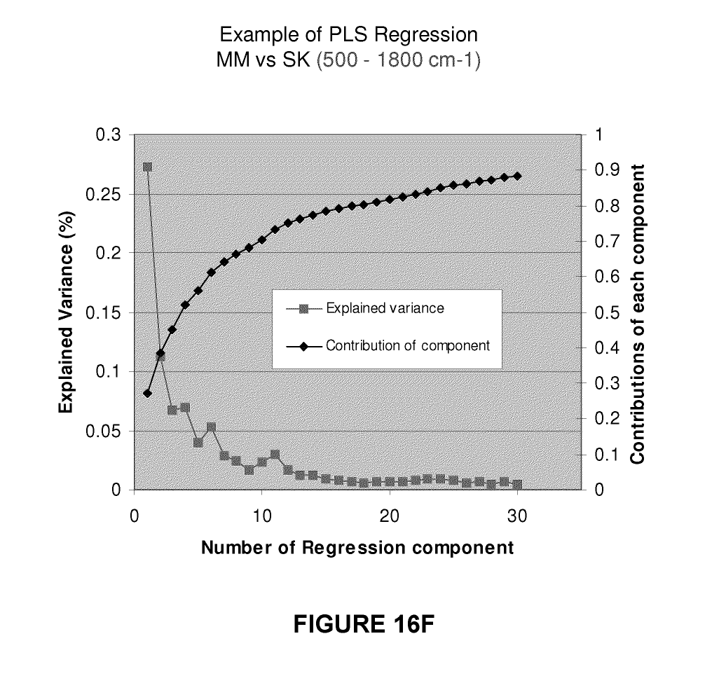

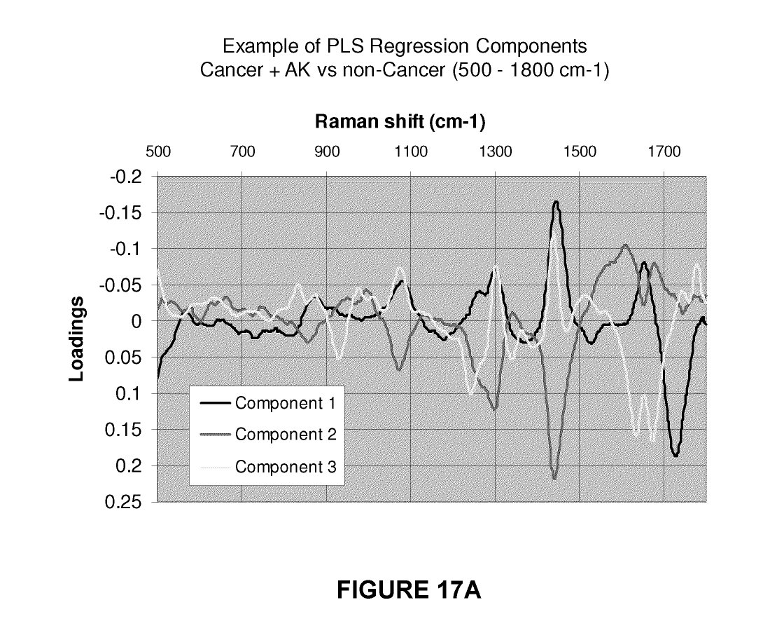

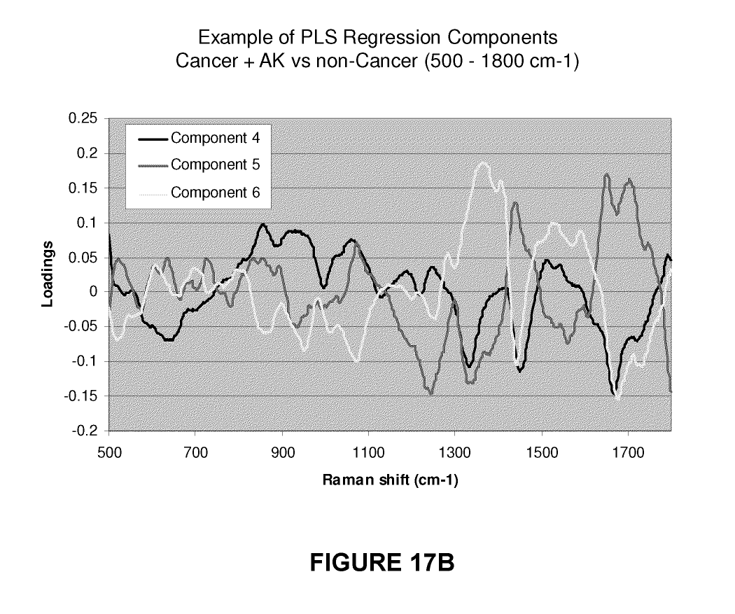

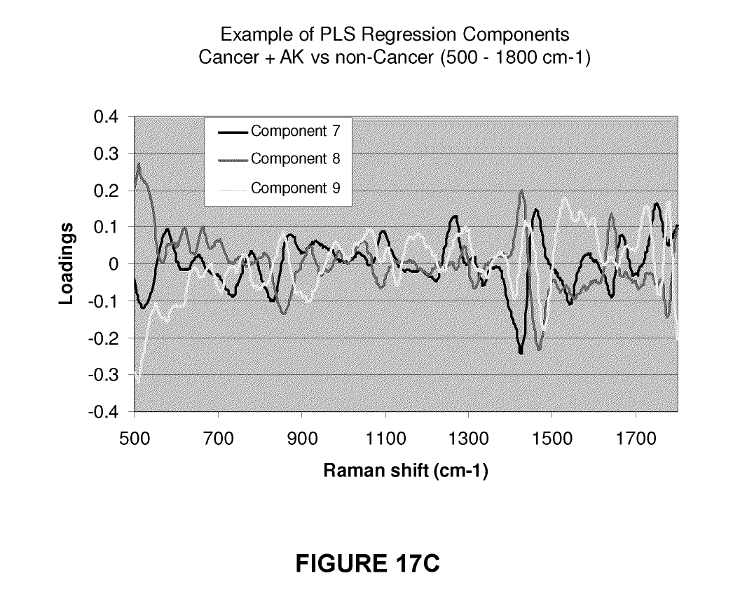

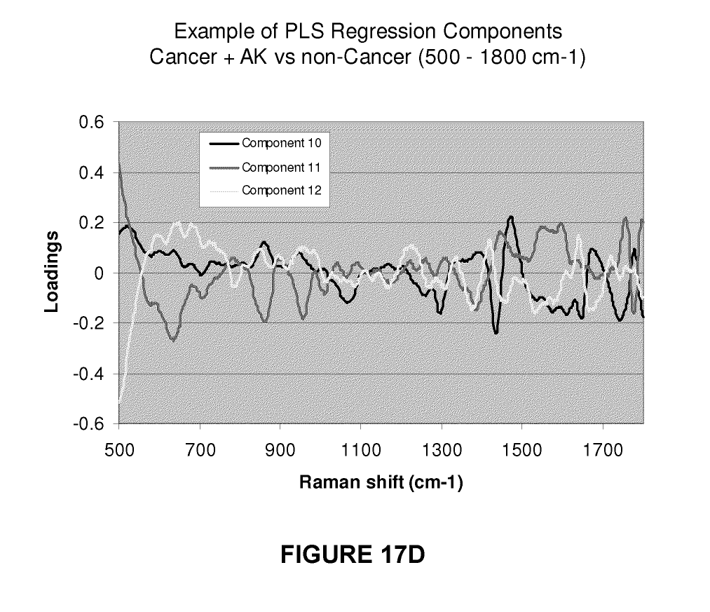

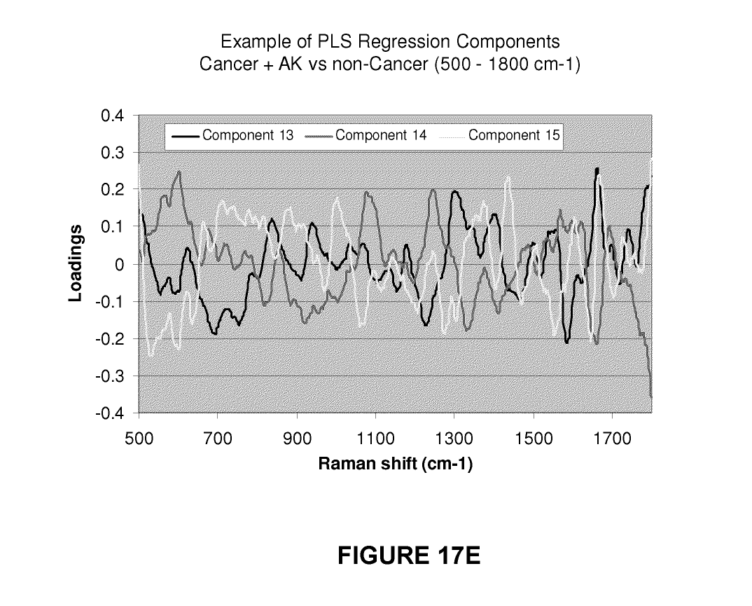

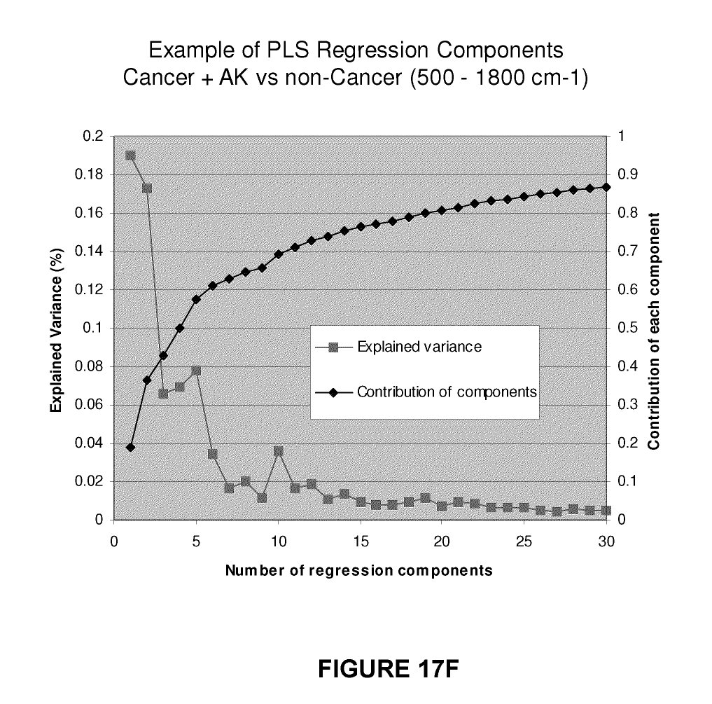

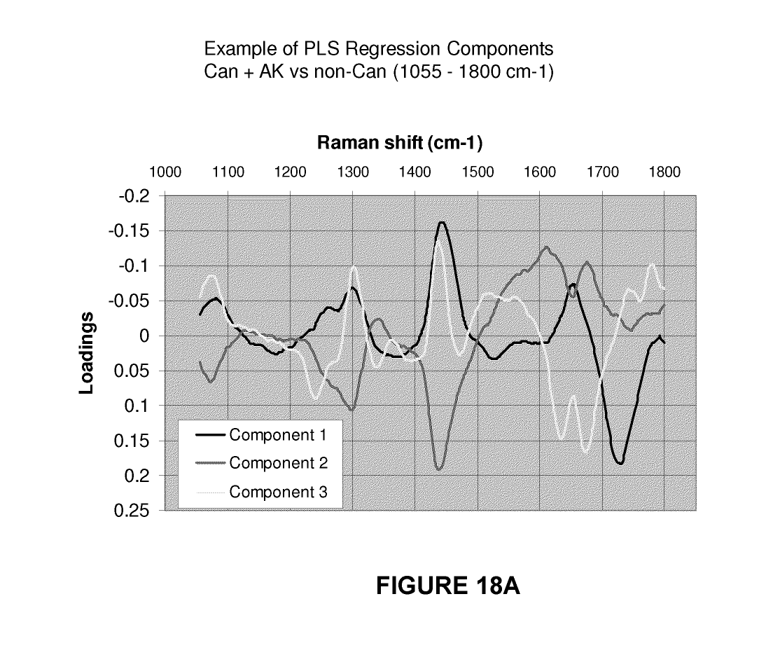

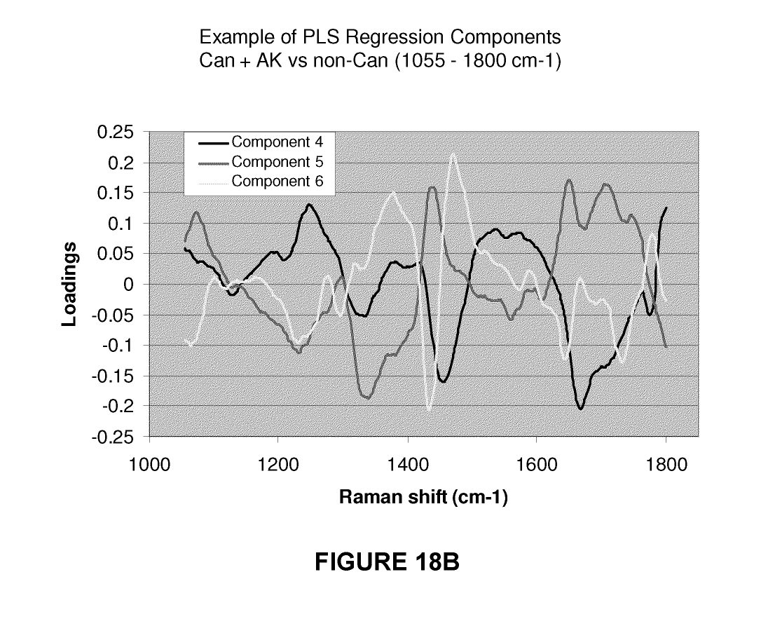

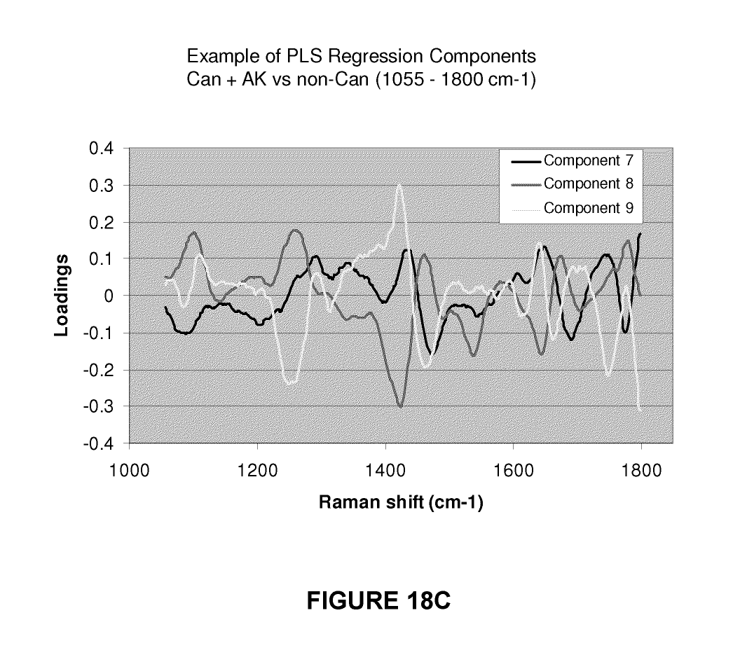

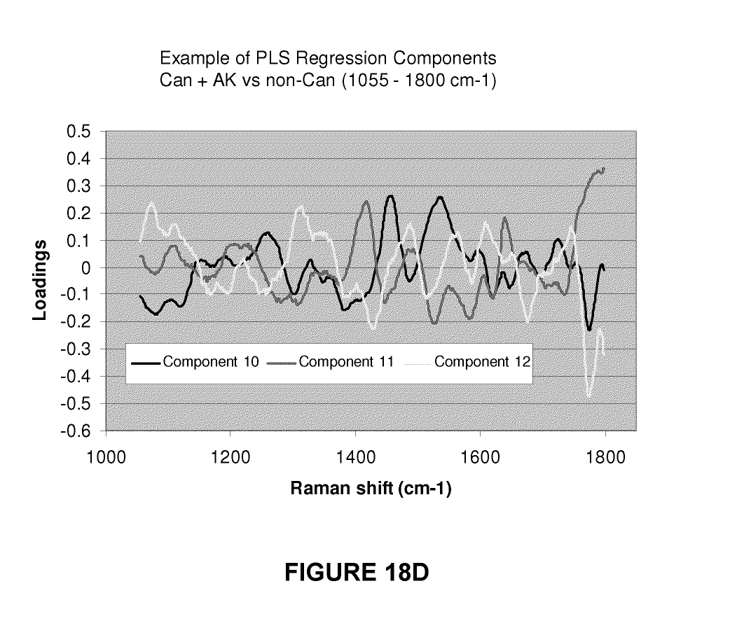

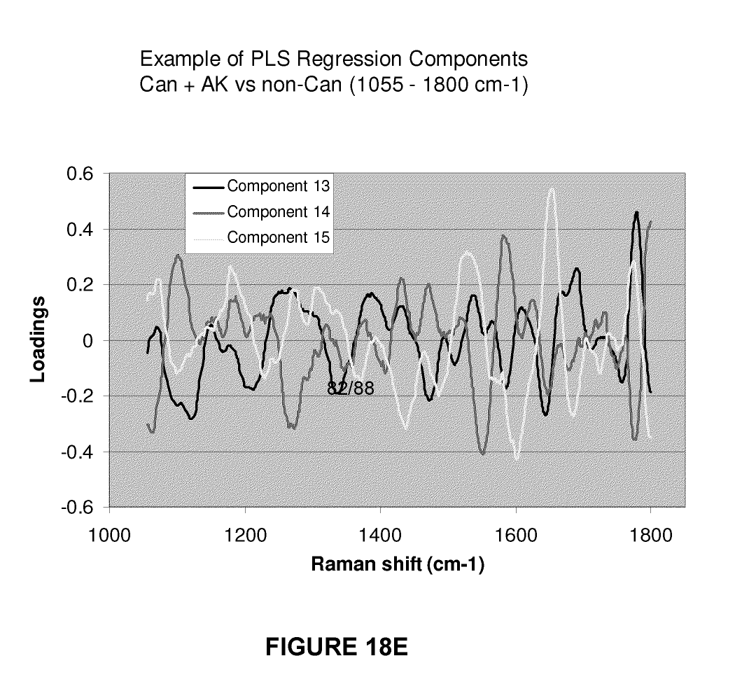

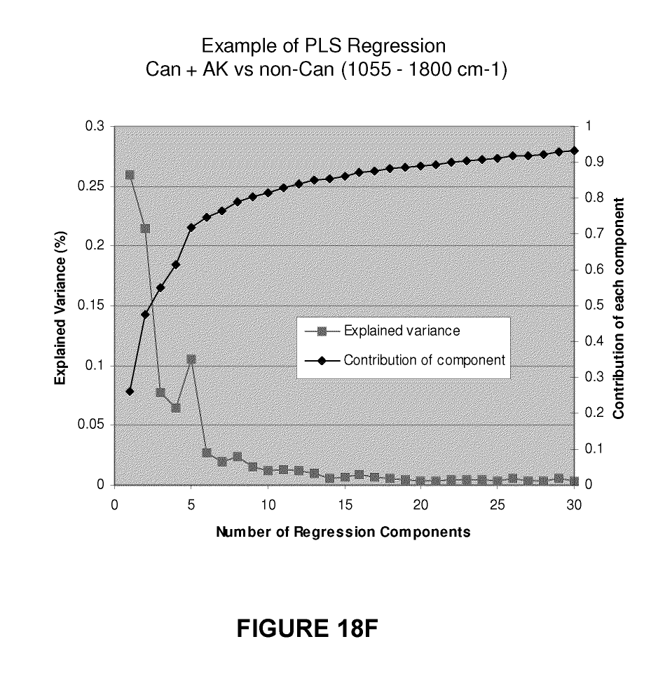

The specific line shapes of PLS factors in the nonlinear iterative partial least squares (NIPLS) analysis also tend to pick up features in the Raman spectrum which distinguish different pathologies. FIGS. 13A to 13E show PLS factors generated for discriminating MM from non-melanoma pigmented lesions using Raman spectra in the range of 1055 cm⁻¹ to 1800 cm⁻¹. FIG. 13F illustrates how the explained variance increases with the number of PLS regression components used. FIGS. 14A to 14E show PLS factors generated for discriminating MM from non-melanoma pigmented lesions using Raman spectra in the range of 500 cm⁻¹ to 1800 cm⁻¹. FIG. 14F illustrates how the explained variance increases with the number of PLS regression components used. FIGS. 15A to 15E show PLS factors generated for discriminating MM from SK using Raman spectra in the range of 1055 cm⁻¹ to 1800 cm⁻¹. FIG. 15F illustrates how the explained variance increases with the number of PLS regression components used. FIGS. 16A to 16E show PLS factors generated for discriminating MM from SK using Raman spectra in the range of 500 cm⁻¹ to 1800 cm⁻¹. FIG. 16F illustrates how the explained variance increases with the number of PLS regression components used. FIGS. 17A to 17E show PLS factors generated for discriminating cancer and pre-cancer (AK) from non-cancer using Raman spectra in the range of 500 cm⁻¹ to 1800 cm⁻¹. FIG. 17F illustrates how the explained variance increases with the number of PLS regression components used. FIGS. 18A to 18E show PLS factors generated for discriminating cancer and pre-cancer (AK) from non-cancer using Raman spectra in the range of 1055 cm⁻¹ to 1800 cm⁻¹. FIG. 18F illustrates how the explained variance increases with the number of PLS regression components used.

The innovations described above may be applied individually or in any appropriate combinations. For example, in some embodiments, measurements of one or more specific Raman peaks are used in combination with other measures, such as PC weightings or least-squares component weightings to generate an indicator of discrimination or classification

Some embodiments optionally apply PCs that differ from but are substantially the same as the PCs illustrated in the accompanying FIGS. 7A-7E, 8A-8E, 9A-9E, 10A-10E, 11A-11E, and 12A-12E. For example, the embodiments may apply PCs that, when normalized, at any wavenumber in the range of the figure, differ from the illustrated PCs by no more than 0.0025, or 0,005, or 0.001, or 0.025, or 0.05. In other example embodiments, the L² norm of the difference between the applied PC and the illustrated PC, over the domain of the illustrated PC, does not exceed 0.00037, or 0.0037, or 0.037, or 0.37, or 0.75, or 1.12.

Some embodiments optionally apply PLS factors that differ from but are substantially the same as the PLS factors illustrated in the accompanying FIGS. 13A-13E, 14A-14E, 15A-15E, 16A-16E, 17A-17E, and 18A-18E. For example, the embodiments may apply PLS factors that, when normalized, at any wavenumber in the range of the figure, differ from the illustrated PLS factors by no more than 0.0025, or 0,005, or 0.001, or 0.025, or 0.05. In other example embodiments, the L² norm of the difference between the applied LPS factors and the illustrated PLS factors, over the domain of the illustrated PLS factors, does not exceed 0.00037, or 0.0037, or 0.037, or 0.37, or 0.75, or 1.12.

### Experimental Results

Experiments were conducted by analyzing Raman spectra from the data set described in Table Ito assess the efficacy of the methods described herein. The experiments demonstrated that the diagnosis capability of Raman spectroscopy is reliable and repeatable.

One experiment was conducted to test the ability of Raman spectroscopy to discriminate melanoma and non-melanoma skin cancers from other benign skin lesions. This is a different approach from some prior studies whose objectives were to discriminate melanoma or non-melanoma skin cancers from normal skin. The experiments demonstrated that similar Raman peaks are present in skin lesions and normal skin. However, the relative intensities of different Raman peaks vary among skin lesions as shown, for example in FIG. 5. This variation provides a basis for discriminating skin cancers from other skin diseases. For different pathologies the Raman spectra have different combinations of features that can be characterized, for example, by multivariate analysis such as PCA-GDA analysis or PLS analysis.

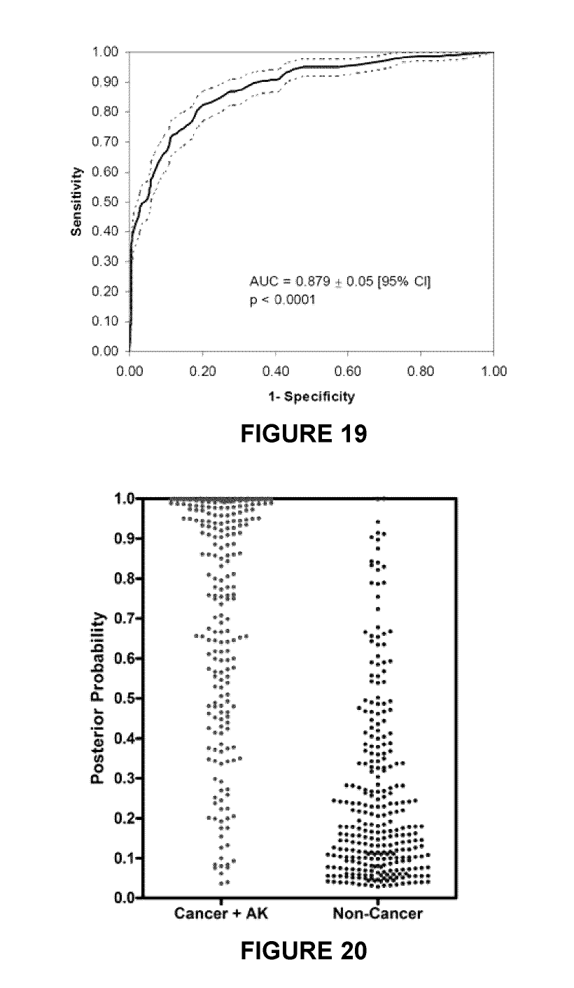

PCA-GDA analysis and PLS analysis were applied to distinguish cancerous and precancerous skin conditions requiring treatment (cancer and AK) from benign skin lesions (non-cancer). The analysis was applied to Raman spectra for 232 cases identified as having the pathology cancer or AK and Raman spectra for 286 cases identified as being benign lesions. FIG. 19 shows a ROC plot for the results of this analysis. The area-under-curve value was 0.879 (95% CI: 0.829-0.929, PCA-GDA) that is statistically significant (p<0.001). At a sensitivity of 90%, the overall specificity is over 64%, with a positive predictive value (PPV) of 67% and a negative predictive value (NPV) of 89%. The estimated biopsy ratio is 0.5:1. FIG. 20 shows the posterior probability for each measured lesion to be classified as a skin cancer or precancer. It can be seen that most of the cancerous or precancerous cases have higher posterior probabilities while most of the benign cases have lower posterior probability.

Other experiments were conducted to test the ability of Raman spectroscopy to discriminate melanoma from other pigmented skin lesions and to test the ability of Raman spectroscopy to discriminate melanoma from seborrheic keratosis. Table 2A reports the corresponding parameters (specificity, positive predictive value (PPV), negative predictive value (NPV), and biopsy ratio) for these three discriminations performed using PC analysis techniques for specificity levels of 90, 95, and 99%. Table 2B reports the same parameters for the three discriminations performed using a PLS analysis.

TABLE 2A
PCA-GDA Analysis Results

| | Sensitivity | | | | biopsy |
|---|---|---|---|---|---|
| Diagnosis | (95% CI) | Specificity | ppv | npv | ratio |
| Cancer + | 0.99 | 0.17 | 0.49 | 0.95 | 1.03:1 |
| AK vs | (0.98-1.00) | (0.13-0.21) | | | |
| NonCan | 0.95 | 0.41 | 0.57 | 0.91 | 0.77:1 |
| | (0.92-0.99) | (0.35-0.48) | | | |
| | 0.90 | 0.64 | 0.67 | 0.89 | 0.49:1 |
| | (0.86-0.94) | (0.58-0.70) | | | |
| MM vs | 0.99 | 0.15 | 0.15 | 0.99 | 5.58:1 |
| PIG | (0.96-1.00) | (0.11-0.19) | | | |
| | 0.95 | 0.38 | 0.19 | 0.98 | 4.24:1 |
| | (0.89-1.00) | (0.32-0.44) | | | |
| | 0.90 | 0.68 | 0.30 | 0.98 | 2.31:1 |
| | (0.81-0.99) | (0.63-0.73) | | | |
| MM vs SK | 0.99 | 0.25 | 0.34 | 0.98 | 1.96:1 |
| | (0.96-1.00) | (0.17-0.33) | | | |
| | 0.95 | 0.54 | 0.44 | 0.97 | 1.25:1 |
| | (0.89-1.00) | (0.45-0.63) | | | |
| | 0.90 | 0.68 | 0.52 | 0.95 | 0.92:1 |
| | (0.81-0.99) | (0.59-0.77) | | | |

TABLE 2B
PLS Analysis Results

| | Sensitivity | | | | biopsy |
|---|---|---|---|---|---|
| Diagnosis | (95% CI) | Specificity | ppv | npv | ratio |
| Cancer + | 0.99 | 0.24 | 0.51 | 0.97 | 0.95:1 |
| AK vs | (0.98-1.00) | (0.19-0.29) | | | |
| NonCan | 0.95 | 0.52 | 0.62 | 0.93 | 0.62:1 |
| | (0.92-0.99) | (0.48-0.58) | | | |
| | 0.90 | 0.66 | 0.68 | 0.89 | 0.47:1 |
| | (0.86-0.94) | (0.61-0.71) | | | |
| MM vs | 0.99 | 0.14 | 0.15 | 0.99 | 5.65:1 |
| PIG | (0.96-1.00) | (0.10-0.18) | | | |
| | 0.95 | 0.44 | 0.21 | 0.98 | 3.83:1 |
| | (0.89-1.00) | (0.38-0.50) | | | |
| | 0.90 | 0.63 | 0.27 | 0.98 | 2.67:1 |
| | (0.81-0.99) | (0.57-0.69) | | | |
| MM vs SK | 0.99 | 0.46 | 0.41 | 0.99 | 1.41:1 |
| | (0.96-1.00) | (0.37-0.55) | | | |
| | 0.95 | 0.52 | 0.43 | 0.96 | 1.31:1 |
| | (0.89-1.00) | (0.43-0.61) | | | |
| | 0.90 | 0.66 | 0.51 | 0.94 | 0.98:1 |
| | (0.81-0.99) | (0.57-0.75) | | | |

The sensitivity and selectivity of a test designed to perform discrimination can depend on what pathologies are classified into each of the groups being discriminated between. Because the diagnosis and treatment of AK are distinct from the diagnosis and treatment of MM, BCC and SCC, an alternative to including AK with cancer is to include AK in the benign category. To demonstrate the ability of the techniques described herein to distinguish MM, BCC and SCC from non-skin cancers (including AK) a discrimination function was generated for these groups of pathologies. The AUC of the ROC curve for the discrimination of skin cancers (MM, BCC, SCC) from non-skin cancers (AN, BN, CN, IN, JN, SK, AK) is 0.863 (95% CI: 0.830-0.895, p<0.001), slightly lower than the results obtained by classifying AK with MM, BCC, and SCC. For a sensitivity of 90%, the overall specificity is over 63%, with a PPV of 60%, NPV of 91% and biopsy ratio of 0.7:1. Raman spectroscopy can detect cancerous skin lesions well irrespective of whether or not AK are included with benign lesions or with cancerous lesions.

As noted above, the technology as described herein may be applied to discriminate between different types of pigmented lesions. It was found that the 44

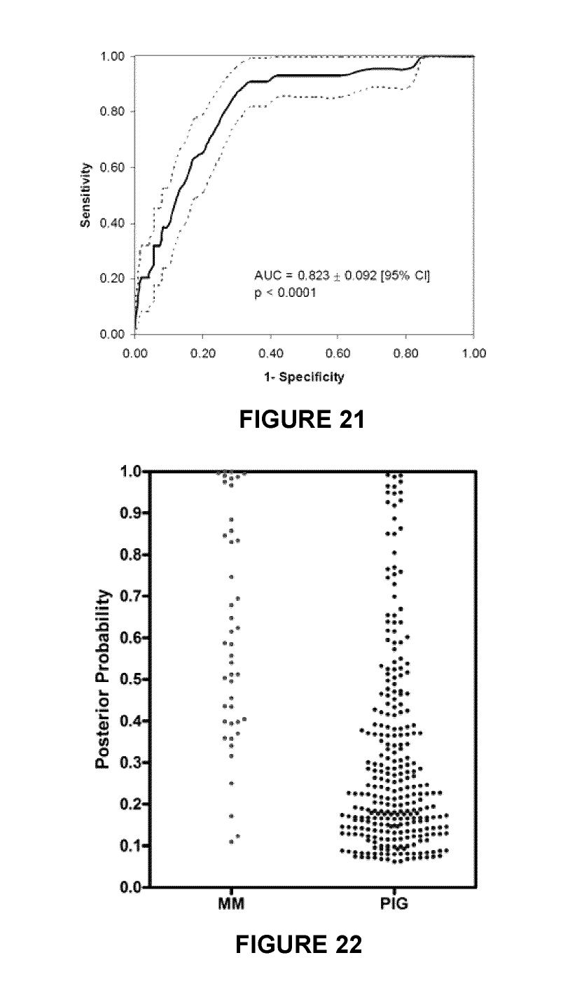

MM cases for which Raman spectra were obtained could be distinguished from the 286 non-melanoma pigmented skin lesions (AN, BN, CN, IN, JN, SK) with an ROC AUC of 0.823 (95% CI: 0.731-0.915, p<0.001). The biopsy ratio based on Raman spectroscopy ranged from 5.6:1 to 2.3:1 for sensitivities corresponding to 99% to 90% and specificities from 15% to 68% respectively. The results are shown in FIGS. 21 and 22.

PCA-GDA and PLS analyses were performed using three different Raman bands (500-1055 cm⁻¹, 1055-1800 cm⁻¹ and 500-1800 cm⁻¹). It was found that the spectral range from 1055 to 1800 cm⁻¹ performed best for differentiation of melanoma from non-melanoma pigmented lesions and for differentiation of melanoma from seborrheic keratosis. The full spectral range from 500 to 1800 cm⁻¹ was found to be best for differentiation of skin cancers and/or precancers from benign skin lesions.

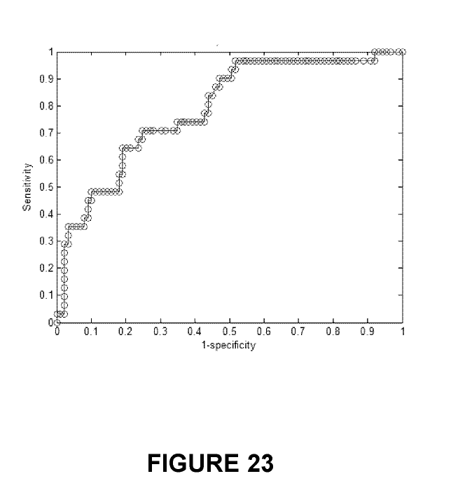

To check whether locations of the lesions affect the ability of methods as described herein, a set of PCs were generated based on the spectra of head lesions only (see Table I) and a discrimination function was generated for PC scores for these PCs. The resulting PCs and discrimination function were applied in an attempt to discriminate Raman spectra for 31 cases of malignant melanoma (MM) from Raman spectra for 89 cases of non-melanoma pigmented lesions (AN, BN, CN, IN, JN, SK). The resulting ROC curve is shown in FIG. 23. The AUC of the ROC curve is 0.789 (95% CI: 0.698-0.879), which is not greatly different from the AUC of 0.823 obtained for the same discrimination using Raman spectra from all body sites to generate the PCs and discrimination function as shown in FIG. 21.

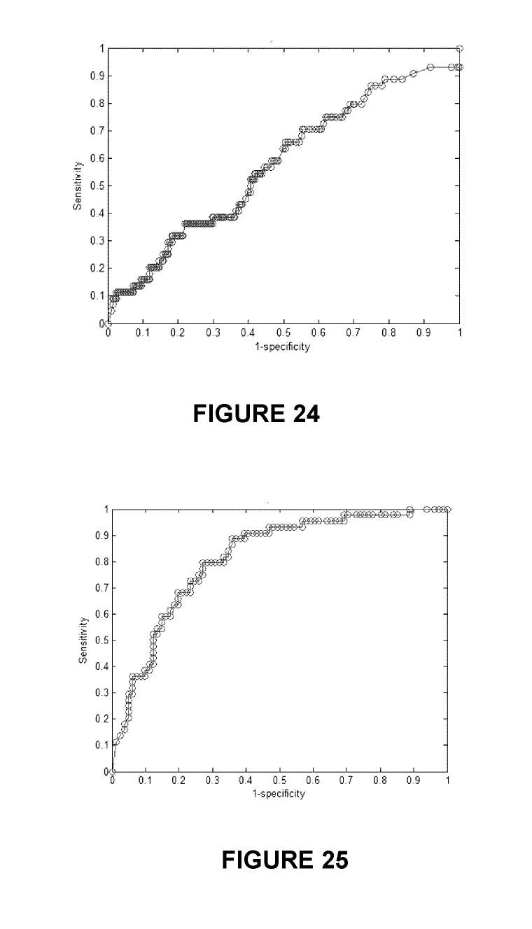

For comparison purposes, the differences between the Raman spectra for the lesions and adjacent normal skin were used in a PCA-GDA analysis to discriminate MM from non-melanoma pigmented skin lesions. It was found that the results were poorer than the results obtained when using the Raman spectra of the lesions alone in the same PCA-GDA analysis. For example, the AUC of the ROC curve for discriminating 44 cases of malignant melanoma (MM) from 286 cases of non-melanoma pigmented lesions (AN, BN, CN, IN, JN, SK) is only 0.577 (95% CI: 0.500-0.670) when using the differences between the Raman spectra for the lesions and adjacent normal skin (see FIG. 24).

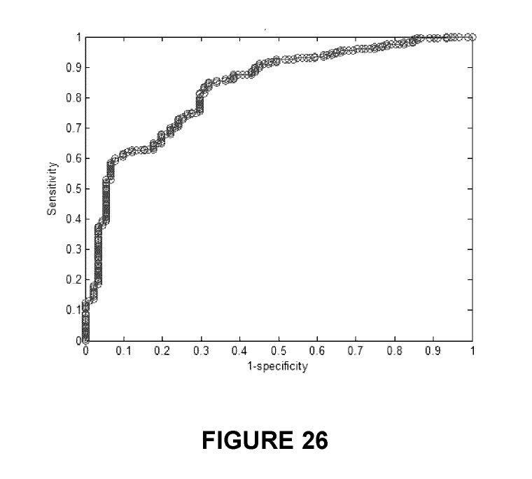

As shown in Table I, all skin cancer cases were biopsied and clinically confirmed by a dermatologist. However, biopsies were not performed on all of the benign lesions represented in the data of Table I. The pathologies of the majority of the benign lesions were verified by visual inspection by the dermatologist. Two experiments were performed using only Raman spectra for lesions in which the pathology had been verified by biopsy. One experiment tested the ability to discriminate biopsied malignant melanoma (MM, n=44) from biopsied non-melanoma pigmented lesions (AN, BN, CN, IN, JN, SK, n=81). This experiment found that the AUC of the ROC curve was 0.813 (95% CI: 0.761-0.906, see FIG. 25), which is very close to the AUC obtained when all cases with/without biopsy were used in the analysis. Another experiment tested the ability to discriminate biopsied skin cancers (MM, SCC, BCC, n=200) from biopsied non-cancerous lesions (AN, BN, CN, IN, JN, SK, AK, n=91). The AUC of the ROC curve based on biopsied spectrum was found to be 0.833 (95% CI: 0.783-0.882, See FIG. 26), which is very close to the AUC obtained when all cases with/without biopsy were used in the analysis.

### Example Application

A patient visits a general practitioner physician (GP) and asks about a skin lesion. The lesion appears somewhat suspicious but the appearance is ambiguous enough that the GP cannot clearly identify the lesion as being cancerous. The GP has apparatus as described above that is configured for distinguishing cancerous tissues from tissues affected by benign lesions (the configuration may be pre-set or built into the apparatus or the GP may select a mode that provides this configuration from a number of available modes). A discrimination function may be set to provide high sensitivity for cancer and somewhat reduced specificity. The GP acquires a Raman spectrum of the suspicious lesion by placing a probe against the lesion and activating the apparatus. The apparatus processes the Raman spectrum as described above and displays an indicator relevant to the discrimination between cancer and benign.

In this example case, the indicator indicates that the lesion may be cancerous and so the GP refers the patient to a specialist (e.g. a dermatologist). Such apparatus which provides a rapid and simple to use test for distinguishing cancerous tissues from tissues affected by benign lesions is useful particularly for general practitioners (GPs), nurse practitioners or end users. For these groups of users, a common concern is to determine whether a suspicious skin lesion is likely enough to be a cancer or precancer that further medical follow up should be done. Unaided clinical diagnosis of skin cancers and precancers by non-specialists is pretty low. For example, some studies have shown a sensitivity of 63.9% for BCC, 41.1% for SCC, and 33.8% for MM with positive predictive values of 72.7% for BCC, 49.4% for SCC and 33.3% for MM. By contrast, using the methodologies described herein, real-time Raman spectroscopy is very effective in differentiation of skin cancer and pre-cancers from benign skin lesions with an overall area under the ROC curve of 0.879 (95% CI 0.829-0.929).

The patient visits a dermatologist who has apparatus as described herein. The dermatologist first identifies a pigmented lesion. The dermatologist considers that she cannot rule out a diagnosis of MM from the appearance of the lesion but that a conclusive diagnosis based on inspection of the lesion is not possible. The dermatologist sets the apparatus in a mode for distinguishing between and MM and benign pigmented lesions, places the probe against the lesion and acquires one or more Raman spectra for tissue in the lesion. The apparatus processes the Raman spectrum as described herein and generates an indication of the discrimination. In this example case, the indication is that the Raman spectrum corresponds to a benign pigmented lesion. In combination with the inconclusive visual appearance of the lesion, the dermatologist decides that it is not necessary to biopsy the lesion. Instead the dermatologist schedules a follow-up appointment with the patient in a few months. If the indicator had indicated that the Raman spectrum corresponded to MM or was inconclusive, the dermatologist would have taken a biopsy of the lesion in this example.

Certain implementations of the invention comprise computer processors which execute software instructions which cause the processors to perform a method of the invention. For example, one or more processors in a medical Raman specrometer may implement methods as described herein by executing software instructions in a program memory accessible to the processors. Discrimination functions may be provided in the software. Data (such as predetermined PCSs or PLS factors, for example), may be provided on a memory accessible to the processors. The invention may also be provided in the form of a program product. The program product may comprise any non-transitory medium which carries a set of computer-readable signals comprising instructions which, when executed by a data processor, cause the data processor to execute a method of the invention. Program products according to the invention may be in any of a wide variety of forms. The program product may comprise, for example, physical media such as magnetic data storage media including floppy diskettes, hard disk drives, optical data storage media including CD ROMs, DVDs, electronic data storage media including ROMs, flash RAM, or the like. The computer-readable signals on the program product may optionally be compressed or encrypted.

Where a component (e.g. a software module, processor, assembly, device, circuit, etc.) is referred to above, unless otherwise indicated, reference to that component (including a reference to a “means”) should be interpreted as including as equivalents of that component, any component which performs the function of the described component (i.e., that is functionally equivalent), including components which are not structurally equivalent to the disclosed structure which perform the function in the illustrated exemplary embodiments of the invention.

As will be apparent to those skilled in the art in the light of the foregoing disclosure, many alterations and modifications are possible in the practice of this invention without departing from the spirit or scope thereof. For example, methods and apparatus as described herein may take into account additional information to provide refined indicators. For example, such methods and apparatus may additionally take into account information from measurements of tissue fluorescence (either in the background of the Raman signal or not). In some embodiments such methods and apparatus may also take into account information such as the presence of a family history or personal history of cancer. For example, if a user indicates by way of a user interface that such a history exists, the apparatus may automatically select configuration providing a higher sensitivity for cancer. Accordingly, the scope of the invention is to be construed in accordance with the substance defined by the following claims.

1. A method for evaluating pathology of a living tissue, the method comprising:
obtaining a Raman spectrum for the tissue;
deriving a first indicator indicating which of a first pair of pathologies the tissue is most likely to be affected by based on a first range of the Raman spectrum; and,
deriving a second indicator indicating which of a second pair of pathologies the tissue is most likely to be affected by based on a second range of the Raman spectrum different from the first range.

2. A method according to claim 1 wherein the first range includes Raman shifts between 500 and 1800 cm⁻¹.

3. (canceled)

4. A method according to claim 2 wherein the second range does not include Raman shifts having wavenumbers of less than 1000 cm⁻¹.

5-6. (canceled)

7. A method according to claim 4 wherein the second indicator indicates discrimination between melanoma and non-melanoma pigmented lesions or discrimination between melanoma from seborrheic keratosis.

8. A method according to claim 4 wherein the first indicator indicates discrimination between skin cancers and benign skin lesions.

9. (canceled)

10. A method according to claim 1 wherein the first indicator indicates discrimination between skin cancers and pre-cancers, on one hand, and benign skin lesions on another hand.

11-12. (canceled)

13. A method according to claim 1 wherein
determining the first indicator comprises determining PC scores for a plurality of predetermined principal components and applying a general determinant analysis to the PC scores;
the first indicator indicates discrimination between MM and SK;
the first range is the range of 1055 cm⁻¹ to 1800 cm⁻¹; and
the principal components include two or more principal components substantially as shown in FIGS. 7A through 7E.

14. A method according to claim 1 wherein
determining the first indicator comprises determining PC scores for a plurality of predetermined principal components and applying a general determinant analysis to the PC scores;
the first indicator indicates discrimination between MM and SK;
the first range is the range of 500 cm⁻¹ to 1800 cm⁻¹; and
the principal components include two or more principal components substantially as shown in FIGS. 8A through 8E.

15. A method according to claim 1 wherein
determining the first indicator comprises determining PC scores for a plurality of predetermined principal components and applying a general determinant analysis to the PC scores;
the first indicator indicates discrimination between cancer including pre-cancer (AK) and non-cancer;
the first range is the range of 500 cm⁻¹ to 1800 cm⁻¹; and
the principal components include two or more principal components substantially as shown in FIGS. 9A through 9E.

16. A method according to claim 1 wherein
determining the first indicator comprises determining PC scores for a plurality of predetermined principal components and applying a general determinant analysis to the PC scores;
the first indicator indicates discrimination between cancer including pre-cancer (AK) and non-cancer;
the first range is the range of 1055 cm⁻¹ to 1800 cm⁻¹; and
the principal components include two or more principal components substantially as shown in FIGS. 10A through 10E.

17. A method according to claim 1 wherein
determining the first indicator comprises determining PC scores for a plurality of predetermined principal components and applying a general determinant analysis to the PC scores;
the first indicator indicates discrimination between MM and non-melanoma pigmented lesions;
the first range is the range of 1055 cm⁻¹ to 1800 cm⁻¹; and
the principal components include two or more principal components substantially as shown in FIGS. 11A through 11E.

18. A method according to claim 1 wherein
determining the first indicator comprises determining PC scores for a plurality of predetermined principal components and applying a general determinant analysis to the PC scores;
the first indicator indicates discrimination between MM and non-melanoma pigmented lesions;
the first range is the range of 500 cm⁻¹ to 1800 cm⁻¹; and
the principal components include two or more principal components substantially as shown in FIGS. 12A through 12E.

19. (canceled)

20. A method according to claim 1 wherein
determining the first indicator comprises determining scores for a plurality of predetermined PLS factors;
the first indicator indicates discrimination between MM and non-melanoma pigmented lesions;
the first range is the range of 1055 cm⁻¹ to 1800 cm⁻¹; and
the PLS factors include two or more PLS factors substantially as shown in FIGS. 13A through 13E.

21. A method according to claim 1 wherein
determining the first indicator comprises determining scores for a plurality of predetermined PLS factors;
the first indicator indicates discrimination between MM and non-melanoma pigmented lesions;
the first range is the range of 500 cm⁻¹ to 1800 cm⁻¹; and
the PLS factors include two or more PLS factors substantially as shown in FIGS. 14A through 14E.

22. A method according to claim 1 wherein
determining the first indicator comprises determining scores for a plurality of predetermined PLS factors;
the first indicator indicates discrimination between MM and SK;
the first range is the range of 1055 cm⁻¹ to 1800 cm⁻¹; and
the PLS factors include two or more PLS factors substantially as shown in FIGS. 15A through 15E.

23. A method according to claim 1 wherein
determining the first indicator comprises determining scores for a plurality of predetermined PLS factors;
the first indicator indicates discrimination between MM and SK;
the first range is the range of 500 cm⁻¹ to 1800 cm⁻¹; and
the PLS factors include two or more PLS factors substantially as shown in FIGS. 16A through 16E.

24. A method according to claim 1 wherein
determining the first indicator comprises determining scores for a plurality of predetermined PLS factors;
the first indicator indicates discrimination between cancer including pre-cancer (AK) from non-cancer;
the first range is the range of 500 cm⁻¹ to 1800 cm⁻¹; and
the PLS factors include two or more PLS factors substantially as shown in FIGS. 17A through 17E.

25. A method according to claim 1 wherein
determining the first indicator comprises determining scores for a plurality of predetermined PLS factors;
the first indicator indicates discrimination between cancer including pre-cancer (AK) from non-cancer;
the first range is the range of 1055 cm⁻¹ to 1800 cm⁻¹; and
the PLS factors include two or more PLS factors substantially as shown in FIGS. 18A through 18E.

26-27. (canceled)

28. A method according to claim 1 wherein the second indicator indicates whether a tissue is more likely affected by malignant melanoma, on one hand, and other pigmented lesions, on the other hand, wherein the second range of the Raman spectrum comprises the Raman spectrum for at least a majority of the following sub-ranges 1055-1100, 1292-1322, 1357-1414, 1426-1480, 1617-1644, 1672-1721, and 1769-1787 cm⁻¹; and,
the method comprises generating the second indicator based upon the values of the Raman spectrum in the sub-ranges while excluding values of the Raman spectrum outside of the sub-ranges.

29. A method according to claim 1 wherein the second indicator indicates whether a tissue is more likely affected by malignant melanoma, on one hand, and seborreic keratosis, on the other hand, wherein the second range of the Raman spectrum comprises the Raman spectrum for at least a majority of the following sub-ranges: 1055-1106, 1143-1147, 1255-1263, 1288-1322, 1343-1416, 1428-1497, 1591-1649, 1665-1736, and 1760-1791 cm⁻¹; and,
the method comprises generating the second indicator based upon the values of the Raman spectrum in the sub-ranges while excluding values of the Raman spectrum outside of the sub-ranges.

30-89. (canceled)
